SEQUENCE LISTING (120 sequences)

SEQ ID NO: 1 - DNA, 32759 bp, synthetic construct
  source 1..32759: mol_type = other DNA
tctcacagtgtacggacctaaagttcccccatagggggtacctaaagcccagccaatcacctaaagtcaaccttcggttgaccttgagggttccctaagggttggggatgacccttgggtttgtctttgggtgttaccttgagtgtctctctgtgtccctatctgttacagtctcctaaagtatcctcctaaagtcacctcctaacgtccatcctaaagccaacacctaaagcctacacctaaagacccatcaagtcaacgcctatcttaaagtttaaacataaagaccagacctaaagaccagacctaaagacactacataaagaccagacctaaagacgccttgttgttagccataaagtgataacctttaatcattgtctttattaatacaactcactataaggagagacaacttaaagagacttaaaagattaatttaaaatttatcaaaaagagtattgacttaaagtctaacctataggatacttacagccatcgagagggacacggcgaatagccatcccaatcgacaccggggtcaaccggataagtagacagcctgataagtcgcacgaaaaacaggtattgacaacatgaagtaacatgcagtaagatacaaatcgctaggtaacactagcagcgtcaaccgggcgcacagtgccttctaggtgacttaagcgcaccacggcacataaggtgaaacaaaacggttgacaacatgaagtaaacacggtacgatgtaccacatgaaacgacagtgagtcaccacactgaaaggtgatgcggtctaacgaaacctgacctaagacgctctttaacaatctggtaaatagctcttgagtgcatgactagcggataactcaagggtatcgcaaggtgccctttatgatattcactaataactgcacgaggtaacacaagatggctatgtctaacatgacttacaacaacgttttcgaccacgcttacgaaatgctgaaagaaaacatccgttatgatgacatccgtgacactgatgacctgcacgatgctattcacatggctgccgataatgcagttccgcactactacgctgacatctttagcgtaatggcaagtgagggcattgaccttgagttcgaagactctggtctgatgcctgacaccaaggacgtaatccgcatcctgcaagcgcgtatctatgagcaattaacgattgacctctgggaagacgcagaagacttgctcaatgaatacttggaggaagtcgaggagtacgaggaggatgaagagtaatgtctactaccaacgtgcaatacggtctgaccgctcaaactgtacttttctatagcgacatggtgcgctgtggctttaactggtcactcgcaatggcacagctcaaagaactgtacgaaaacaacaaggcaatagctttagaatctgctgagtgatagactcaaggtcgctcctagcgagtggcctttatgattatcactttacttatgagggagtaatgtatatgcttactatcggtctactcaccgctctaggtctagctgtaggtgcatcctttgggaaggctttaggtgtagctgtaggttcctactttaccgcttgcatcatcataggaatcatcaaaggggcactacgcaaatgatgaagcactacgttatgccaatccacacgtccaacggggcaaccgtatgtacacctgatgggttcgcaatgaaacaacgaatcgaacgccttaagcgtgaactccgcattaaccgcaagattaacaagataggttccggctatgacagaacgcactgatggcttaaagaaaggttatatgcccaatggcacactatacgctgcaaatcggcgaatagtgagaacttggcgagagaacaacctcgaacgccgcaaggacaagagagggcggcgtggcatagacgaaaggaaaaggttaaagccaagaaactcgccgcacttgaacaggcactagccaacacactgaacgctatctcataacgaacataaaggacacaatgcaatgaacattaccgacatcatgaacgctatcgacgcaatcaaagcactgccaatctgtgaacttgacaagcgtcaaggtatgcttatcgacttactggtcgagatggtcaacagcgagacgtgtgatggcgagctaaccgaactaaatcaggcacttgagcatcaagattggtggactaccttgaagtgtctcacggctgacgcagggttcaagatgctcggtaatggtcacttctcggctgcttatagtcacccgctgctacctaacagagtgattaaggtgggctttaagaaagaggattcaggcgcagcctataccgcattctgccgcatgtatcagggtcgtcctggtatccctaacgtctacgatgtacagcgccacgctggatgctatacggtggtacttgacgcacttaaggattgcgagcgtttcaacaatgatgcccattataaatacgctgagattgcaagcgacatcattgattgcaattcggatgagcatgatgagttaactggatgggatggtgagtttgttgaaacttgtaaactaatccgcaagttctttgagggcatcgcctcattcgacatgcatagcgggaacatcatgttctcaaatggagacgtaccatacatcaccgacccggtatcattctcgcagaagaaagacggtggcgcattcagcatcgaccctgaggaactcatcaaggaagtcgaggaagtcgcacgacagaaagaaattgaccgcgctaaggcccgtaaagaacgtcacgaggggcgcttagaggcacgcagattcaaacgtcgcaaccgcaaggcacgtaaagcacacaaagctaagcgcgaaagaatgcttgctgcgtggcgatgggctgaacgtcaagaacggcgtaaccatgaggtagctgtagatgtactaggaagaaccaataacgctatgctctgggtcaacatgttctctggggactttaaggcgcttgaggaacgaatcgcgctgcactggcgtaatgctgaccggatggctatcgctaatggtcttacgctcaacattgataagcaacttgacgcaatgttaatgggctgatagtcttatcttacaggtcatctgcgggtggcctgaataggtacgatttactaactggaagaggcactaaatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaacgccaaatcaatacgactcactatagagggacaaactcaaggtcattcgcaagagtggcctttatgattgaccttcttccggttaatacgactcactataggagaaccttaaggtttaactttaagacccttaagtgttaattagagatttaaattaaagaattactaagagaggactttaagtatgcgtaacttcgaaaagatgaccaaacgttctaaccgtaatgctcgtgacttcgaggcaaccaaaggtcgcaagttgaataagactaagcgtgaccgctctcacaagcgtagctgggagggtcagtaagatgggacgtttatatagtggtaatctggcagcattcaaggcagcaacaaacaagctgttccagttagacttagcggtcatttatgatgactggtatgatgcctatacaagaaaagattgcatacggttacgtattgaggacaggagtggaaacctgattgatactagcaccttctaccaccacgacgaggacgttctgttcaatatgtgtactgattggttgaaccatatgtatgaccagttgaaggactggaagtaatacgactcagtatagggacaatgcttaaggtcgctctctaggagtggccttagtcatttaaccaataggagataaacattatgatgaacattaagactaacccgtttaaagccgtgtctttcgtagagtctgccattaagaaggctctggataacgctgggtatcttatcgctgaaatcaagtacgatggtgtacgcgggaacatctgcgtagacaatactgctaacagttactggctctctcgtgtatctaaaacgattccggcactggagcacttaaacgggtttgatgttcgctggaagcgtctactgaacgatgaccgttgcttctacaaagatggctttatgcttgatggggaactcatggtcaagggcgtagactttaacacagggtccggcctactgcgtaccaaatggactgacacgaagaaccaagagttccatgaagagttattcgttgaaccaatccgtaagaaagataaagttccctttaagctgcacactggacaccttcacataaaactgtacgctatcctcccgctgcacatcgtggagtctggagaagactgtgatgtcatgacgttgctcatgcaggaacacgttaagaacatgctgcctctgctacaggaatacttccctgaaatcgaatggcaagcggctgaatcttacgaggtctacgatatggtagaactacagcaactgtacgagcagaagcgagcagaaggccatgagggtctcattgtgaaagacccgatgtgtatctataagcgcggtaagaaatctggctggtggaaaatgaaacctgagaacgaagctgacggtatcattcagggtctggtatggggtacaaaaggtctggctaatgaaggtaaagtgattggttttgaggtgcttcttgagagtggtcgtttagttaacgccacgaatatctctcgcgccttaatggatgagttcactgagacagtaaaagaggccaccctaagtcaatggggattctttagcccatacggtattggcgacaacgatgcttgtactattaacccttacgatggctgggcgtgtcaaattagctacatggaggaaacacctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagctatcctgacgcttgcgtatattcttgcggtataccctcaagtagcactagtagtagttggcgcttgttacttagcggcagtgtgtgcttgcgtgtggagtatagttaactggtaatacgactcactaaaggaggtacacaccatgatgtacttaatgccattactcatcgtcattgtaggatgccttgcgctccactgtagcgatgatgatatgccagatggtcacgcttaatacgactcactaaaggagacactatatgtttcgacttcattacaacaaaagcgttaagaatttcacggttcgccgtgctgaccgttcaatcgtatgtgcgagcgagcgccgagctaagatacctcttattggtaacacagttcctttggcaccgagcgtccacatcattatcacccgtggtgactttgagaaagcaatagacaagaaacgtccggttcttagtgtggcagtgacccgcttcccgttcgtccgtctgttactcaaacgaatcaaggaggtgttctgatgggactgttagatggtgaagcctgggaaaaagaaaacccgccagtacaagcaactgggtgtatagcttgcttagagaaagatgaccgttatccacacacctgtaacaaaggagctaacgatatgaccgaacgtgaacaagagatgatcattaagttgatagacaataatgaaggtcgcccagatgatttgaatggctgcggtattctctgctccaatgtcccttgccacctctgccccgcaaataacgatcaaaagataaccttaggtgaaatccgagcgatggacccacgtaaaccacatctgaataaacctgaggtaactcctacagatgaccagccttccgctgagacaatcgaaggtgtcactaagccttcccactacatgctgtttgacgacattgaggctatcgaagtgattgctcgttcaatgaccgttgagcagttcaagggatactgcttcggtaacatcttaaagtacagactacgtgctggtaagaagtcagagttagcgtacttagagaaagacctagcgaaagcagacttctataaagaactctttgagaaacataaggataaatgttatgcataacttcaagtcaaccccacctgccgacagcctatctgatgacttcacatcttgctcagagtggtgccgaaagatgtgggaagagacattcgacgatgcgtacatcaagctgtatgaactttggaaatcgagaggtcaatgactatgtcaaacgtaaatacaggttcacttagtgtggacaataagaagttttgggctaccgtagagtcctcggagcattccttcgaggttccaatctacgctgagaccctagacgaagctctggagttagccgaatggcaatacgttccggctggctttgaggttactcgtgtgcgtccttgtgtagcaccgaagtaatacgactcactattagggaagactccctctgagaaaccaaacgaaacctaaaggagattaacattatggctaagaagattttcacctctgcgctgggtaccgctgaaccttacgcttacatcgccaagccggactacggcaacgaagagcgtggctttgggaaccctcgtggtgtctataaagttgacctgactattcccaacaaagacccgcgctgccagcgtatggtcgatgaaatcgtgaagtgtcacgaagaggcttatgctgctgccgttgaggaatacgaagctaatccacctgctgtagctcgtggtaagaaaccgctgaaaccgtatgagggtgacatgccgttcttcgataacggtgacggtacgactacctttaagttcaaatgctacgcgtctttccaagacaagaagaccaaagagaccaagcacatcaatctggttgtggttgactcaaaaggtaagaagatggaagacgttccgattatcggtggtggctctaagctgaaagttaaatattctctggttccatacaagtggaacactgctgtaggtgcgagcgttaagctgcaactggaatccgtgatgctggtcgaactggctacctttggtggcggtgaagacgattgggctgacgaagttgaagagaacggctatgttgcctctggttctgccaaagcgagcaaaccacgcgacgaagaaagctgggacgaagacgacgaagagtccgaggaagcagacgaagacggagacttctaagtggaactgcgggagaaaatccttgagcgaatcaaggtgacttcctctgggtgttgggagtggcagggcgctacgaacaataaagggtacgggcaggtgtggtgcagcaataccggaaaggttgtctactgtcatcgcgtaatgtctaatgctccgaaaggttctaccgtcctgcactcctgtgataatccattatgttgtaaccctgaacacctatccataggaactccaaaagagaactccactgacatggtaaataagggtcgctcacacaaggggtataaactttcagacgaagacgtaatggcaatcatggagtccagcgagtccaatgtatccttagctcgcacctatggtgtctcccaacagactatttgtgatatacgcaaagggaggcgacatggcaggttacggcgctaaaggaatccgaaaggttggagcgtttcgctctggcctagaggacaaggtttcaaagcagttggaatcaaaaggtattaaattcgagtatgaagagtggaaagtgccttatgtaattccggcgagcaatcacacttacactccagacttcttacttccaaacggtatattcgttgagacaaagggtctgtgggaaagcgatgatagaaagaagcacttattaattagggagcagcaccccgagctagacatccgtattgtcttctcaagctcacgtactaagttatacaaaggttctccaacgtcttatggagagttctgcgaaaagcatggtattaagttcgctgataaactgatacctgctgagtggataaaggaacccaagaaggaggtcccctttgatagattaaaaaggaaaggaggaaagaaataatggctcgtgtacagtttaaacaacgtgaatctactgacgcaatctttgttcactgctcggctaccaagccaagtcagaatgttggtgtccgtgagattcgccagtggcacaaagagcagggttggctcgatgtgggataccactttatcatcaagcgagacggtactgtggaggcaggacgagatgagatggctgtaggctctcacgctaagggttacaaccacaactctatcggcgtctgccttgttggtggtatcgacgataaaggtaagttcgacgctaactttacgccagcccaaatgcaatcccttcgctcactgcttgtcacactgctggctaagtacgaaggcgctgtgcttcgcgcccatcatgaggtggcgccgaaggcttgcccttcgttcgaccttaagcgttggtgggagaagaacgaactggtcacttctgaccgtggataattaattgaactcactaaagggagaccacagcggtttccctttgttcgcattggaggtcaaataatgcgcaagtcttataaacaattctataaggctccgaggaggcatatccaagtgtgggaggcagccaatgggcctataccaaaaggttattatatagaccacattgacggcaatccactcaacgacgccttagacaatctccgtctggctctcccaaaagaaaactcatggaacatgaagactccaaagagcaatacctcaggactaaagggactgagttggagcaaggaaagggagatgtggagaggcactgtaacagctgagggtaaacagcataactttcgtagtagagatctattggaagtcgttgcgtggatttatagaactaggagggaattgcatggacaattcgcacgattccgatagtgtatttctttaccacattccttgtgacaactgtgggagtagtgatgggaactcgctgttctctgacggacacacgttctgctacgtatgcgagaagtggactgctggtaatgaagacactaaagagagggcttcaaaacggaaaccctcaggaggtaaaccaatgacttacaacgtgtggaacttcggggaatccaatggacgctactccgcgttaactgcgagaggaatctccaaggaaacctgtcagaaggctggctactggattgccaaagtagacggtgtgatgtaccaagtggctgactatcgggaccagaacggcaacattgtgagtcagaaggttcgagataaagataagaactttaagaccactggtagtcacaagagtgacgctctgttcgggaagcacttgtggaatggtggtaagaagattgtcgttacagaaggtgaaatcgacatgcttaccgtgatggaacttcaagactgtaagtatcctgtagtgtcgttgggtcacggtgcctctgccgctaagaagacatgcgctgccaactacgaatactttgaccagttcgaacagattatcttaatgttcgatatggacgaagcagggcgcaaagcagtcgaagaggctgcacaggttctacctgctggtaaggtacgagtggcagttcttccgtgtaaggatgcaaacgagtgtcacctaaatggtcacgaccgtgaaatcatggagcaagtgtggaatgctggtccttggattcctgatggtgtggtatcggctctttcgttacgtgaacgaatccgtgagcacctatcgtccgaggaatcagtaggtttacttttcagtggctgcactggtatcaacgataagaccttaggtgcccgtggtggtgaagtcattatggtcacttccggttccggtatgggtaagtcaacgttcgtccgtcaacaagctctacaatggggcacagcgatgggcaagaaggtaggcttagcgatgcttgaggagtccgttgaggagaccgctgaggaccttataggtctacacaaccgtgtccgactgagacaatccgactcactaaagagagagattattgagaacggtaagttcgaccaatggttcgatgaactgttcggcaacgatacgttccatctatatgactcattcgccgaggctgagacggatagactgctcgctaagctggcctacatgcgctcaggcttgggctgtgacgtaatcattctagaccacatctcaatcgtcgtatccgcttctggtgaatccgatgagcgtaagatgattgacaacctgatgaccaagctcaaagggttcgctaagtcaactggggtggtgctggtcgtaatttgtcaccttaagaacccagacaaaggtaaagcacatgaggaaggtcgccccgtttctattactgacctacgtggttctggcgcactacgccaactatctgatactattattgcccttgagcgtaatcagcaaggcgatatgcctaaccttgtcctcgttcgtattctcaagtgccgctttactggtgatactggtatcgctggctacatggaatacaacaaggaaaccggatggcttgaaccatcaagttactcaggggaagaagagtcacactcagagtcaacagactggtccaacgacactgacttctgacaggattcttgatgactttccagacgactacgagaagtttcgctggagagtcccattctaatacgactcactaaaggagacacaccatgttcaaactgattaagaagttaggccaactgctggttcgtatgtacaacgtggaagccaagcgactgaacgatgaggctcgtaaagaggccacacagtcacgcgctctggcgattcgctccaacgaactggctgacagtgcatccactaaagttaccgaggctgcccgtgtggcaaaccaagctcaacagctttccaaattctttgagtaatcaaacaggagaaaccattatgtctaacgtagctgaaactatccgtctatccgatacagctgaccagtggaaccgtcgagtccacatcaacgttcgcaacggtaaggcgactatggtttaccgctggaaggactctaagtcctctaagaatcacactcagcgtatgacgttgacagatgagcaagcactgcgtctggtcaatgcgcttaccaaagctgccgtgacagcaattcatgaagctggtcgcgtcaatgaagctatggctatcctcgacaagattgataactaagagtggtatcctcaaggtcgccaaagtggtggccttcatgaatactattcgactcactataggagatattaccatgcgtgaccctaaagttatccaagcagaaatcgctaaactggaagctgaactggaggacgttaagtaccatgaagctaagactcgctccgctgttcacatcttgaagaacttaggctggacttggacaagacagactggctggaagaaaccagaagttaccaagctgagtcataaggtgttcgataaggacactatgacccacatcaaggctggtgattgggttaaggttgacatgggagttgttggtggatacggctacgtccgctcagttagtggcaaatatgcacaagtgtcatacatcacaggtgttactccacgcggtgcaatcgttgccgataagaccaacatgattcacacaggtttcttgacagttgtttcatatgaagagattgttaagtcacgataatcaataggagaaatcaatatgatcgtttctgacatcgaagctaacgccctcttagagagcgtcactaagttccactgcggggttatctacgactactccaccgctgagtacgtaagctaccgtccgagtgacttcggtgcgtatctggatgcgctggaagccgaggttgcacgaggcggtcttattgtgttccacaacggtcacaagtatgacgttcctgcattgaccaaactggcaaagttgcaattgaaccgagagttccaccttcctcgtgagaactgtattgacacccttgtgttgtcacgtttgattcattccaacctcaaggacaccgatatgggtcttctgcgttccggcaagttgcccggaaaacgctttgggtctcacgctttggaggcgtggggttatcgcttaggcgagatgaagggtgaatacaaagacgactttaagcgtatgcttgaagagcagggtgaagaatacgttgacggaatggagtggtggaacttcaacgaagagatgatggactataacgttcaggacgttgtggtaactaaagctctccttgagaagctactctctgacaaacattacttccctcctgagattgactttacggacgtaggatacactacgttctggtcagaatcccttgaggccgttgacattgaacatcgtgctgcatggctgctcgctaaacaagagcgcaacgggttcccgtttgacacaaaagcaatcgaagagttgtacgtagagttagctgctcgccgctctgagttgctccgtaaattgaccgaaacgttcggctcgtggtatcagcctaaaggtggcactgagatgttctgccatccgcgaacaggtaagccactacctaaataccctcgcattaagacacctaaagttggtggtatctttaagaagcctaagaacaaggcacagcgagaaggccgtgagccttgcgaacttgatacccgcgagtacgttgctggtgctccttacaccccagttgaacatgttgtgtttaacccttcgtctcgtgaccacattcagaagaaactccaagaggctgggtgggtcccgaccaagtacaccgataagggtgctcctgtggtggacgatgaggtactcgaaggagtacgtgtagatgaccctgagaagcaagccgctatcgacctcattaaagagtacttgatgattcagaagcgaatcggacagtctgctgagggagacaaagcatggcttcgttatgttgctgaggatggtaagattcatggttctgttaaccctaatggagcagttacgggtcgtgcgacccatgcgttcccaaaccttgcgcaaattccgggtgtacgttctccttatggagagcagtgtcgcgctgcttttggcgctgagcaccatttggatgggataactggtaagccttgggttcaggctggcatcgacgcatccggtcttgagctacgctgcttggctcacttcatggctcgctttgataacggcgagtacgctcacgagattcttaacggcgacatccacactaagaaccagatagctgctgaactacctacccgagataacgctaagacgttcatctatgggttcctctatggtgctggtgatgagaagattggacagattgttggtgctggtaaagagcgcggtaaggaactcaagaagaaattccttgagaacacccccgcgattgcagcactccgcgagtctatccaacagacacttgtcgagtcctctcaatgggtagctggtgagcaacaagtcaagtggaaacgccgctggattaaaggtctggatggtcgtaaggtacacgttcgtagtcctcacgctgccttgaataccctactgcaatctgctggtgctctcatctgcaaactgtggattatcaagaccgaagagatgctcgtagagaaaggcttgaagcatggctgggatggggactttgcgtacatggcatgggtacatgatgaaatccaagtaggctgccgtaccgaagagattgctcaggtggtcattgagaccgcacaagaagcgatgcgctgggttggagaccactggaacttccggtgtcttctggataccgaaggtaagatgggtcctaattgggcgatttgccactgatacaggaggctactcatgaacgaaagacacttaacaggtgctgcttctgaaatgctagtagcctacaaatttaccaaagctgggtacactgtctattaccctatgctgactcagagtaaagaggacttggttgtatgtaaggatggtaaatttagtaaggttcaggttaaaacagccacaacggttcaaaccaacacaggagatgccaagcaggttaggctaggtggatgcggtaggtccgaatataaggatggagactttgacattcttgcggttgtggttgacgaagatgtgcttattttcacatgggacgaagtaaaaggtaagacatccatgtgtgtcggcaagagaaacaaaggcataaaactataggagaaattattatggctatgacaaagaaatttaaagtgtccttcgacgttaccgcaaagatgtcgtctgacgttcaggcaatcttagagaaagatatgctgcatctatgtaagcaggtcggctcaggtgcgattgtccccaatggtaaacagaaggaaatgattgtccagttcctgacacacggtatggaaggattgatgacattcgtagtacgtacatcatttcgtgaggccattaaggacatgcacgaagagtatgcagataaggactctttcaaacaatctcctgcaacagtacgggaggtgttctgatgtctgactacctgaaagtgctgcaagcaatcaaaagttgccctaagactttccagtccaactatgtacggaacaatgcgagcctcgtagcggaggccgcttcccgtggtcacatctcgtgcctgactactagtggacgtaacggtggcgcttgggaaatcactgcttccggtactcgctttctgaaacgaatgggaggatgtgtctaatgtctcgtgaccttgtgactattccacgcgatgtgtggaacgatatacagggctacatcgactctctggaacgtgagaacgatagccttaagaatcaactaatggaagctgacgaatacgtagcggaactagaggagaaacttaatggcacttcttgaccttaaacaattctatgagttacgtgaaggctgcgacgacaagggtatccttgtgatggacggcgactggctggtcttccaagctatgagtgctgctgagtttgatgcctcttgggaggaagagatttggcaccgatgctgtgaccacgctaaggcccgtcagattcttgaggattccattaagtcctacgagacccgtaagaaggcttgggcaggtgctccaattgtccttgcgttcaccgatagtgttaactggcgtaaagaactggttgacccgaactataaggctaaccgtaaggccgtgaagaaacctgtagggtactttgagttccttgatgctctctttgagcgcgaagagttctattgcatccgtgagcctatgcttgagggtgatgacgttatgggagttattgcttccaatccgtctgccttcggtgctcgtaaggctgtaatcatctcttgcgataaggactttaagaccatccctaactgtgacttcctgtggtgtaccactggtaacatcctgactcagaccgaagagtccgctgactggtggcacctcttccagaccatcaagggtgacatcactgatggttactcagggattgctggatggggtgataccgccgaggacttcttgaataacccgttcataaccgagcctaaaacgtctgtgcttaagtccggtaagaacaaaggccaagaggttactaaatgggttaaacgcgaccctgagcctcatgagacgctttgggactgcattaagtccattggcgcgaaggctggtatgaccgaagaggatattatcaagcagggccaaatggctcgaatcctacggttcaacgagtacaactttattgacaaggagatttacctgtggagaccgtagcgtatattggtctgggtctttgtgttctcggagtgtgcctcatttcgtggggcctttgggacttagccagaataatcaagtcgttacacgacactaagtgataaactcaaggtccctaaattaatacgactcactatagggagataggggcctttacgattattactttaagatttaactctaagaggaatctttattatgttaacacctattaaccaattacttaagaaccctaacgatattccagatgtacctcgtgcaaccgctgagtatctacaggttcgattcaactatgcgtacctcgaagcgtctggtcatataggacttatgcgtgctaatggttgtagtgaggcccacatcttgggtttcattcagggcctacagtatgcctctaacgtcattgacgagattgagttacgcaaggaacaactaagagatgatggggaggattgacactatgtgtttctcaccgaaaattaaaactccgaagatggataccaatcagattcgagccgttgagccagcgcctctgacccaagaagtgtcaagcgtggagttcggtgggtcttctgatgagacggataccgagggcaccgaagtgtctggacgcaaaggcctcaaggtcgaacgtgatgattccgtagcgaagtctaaagccagcggcaatggctccgctcgtatgaaatcttccatccgtaagtccgcatttggaggtaagaagtgatgtctgagttcacatgtgtggaggctaagagtcgcttccgtgcaatccggtggactgtggaacaccttgggttgcctaaaggattcgaaggacactttgtgggctacagcctctacgtagacgaagtgatggacatgtctggttgccgtgaagagtacattctggactctaccggaaaacatgtagcgtacttcgcgtggtgcgtaagctgtgacattcaccacaaaggagacattctggatgtaacgtccgttgtcattaatcctgaggcagactctaagggcttacagcgattcctagcgaaacgctttaagtaccttgcggaactccacgattgcgattgggtgtctcgttgtaagcatgaaggcgagacaatgcgtgtatactttaaggaggtataagttatgggtaagaaagttaagaaggccgtgaagaaagtcaccaagtccgttaagaaagtcgttaaggaaggggctcgtccggttaaacaggttgctggcggtctagctggtctggctggtggtactggtgaagcacagatggtggaagtaccacaagctgccgcacagattgttgacgtacctgagaaagaggtttccactgaggacgaagcacagacagaaagcggacgcaagaaagctcgtgctggcggtaagaaatccttgagtgtagcccgtagctccggtggcggtatcaacatttaatcaggaggttatcgtggaagactgcattgaatggaccggaggtgtcaactctaagggttatggtcgtaagtgggttaatggtaaacttgtgactccacataggcacatctatgaggagacatatggtccagttccaacaggaattgtggtgatgcatatctgcgataaccctaggtgctataacataaagcaccttacgcttggaactccaaaggataattccgaggacatggttaccaaaggtagacaggctaaaggagaggaactaagcaagaaacttacagagtcagacgttctcgctatacgctcttcaaccttaagccaccgctccttaggagaactgtatggagtcagtcaatcaaccataacgcgaatactacagcgtaagacatggagacacatttaatggctgagaaacgaacaggacttgcggaggatggcgcaaagtctgtctatgagcgtttaaagaacgaccgtgctccctatgagacacgcgctcagaattgcgctcaatataccatcccatcattgttccctaaggactccgataacgcctctacagattatcaaactccgtggcaagccgtgggcgctcgtggtctgaacaatctagcctctaagctcatgctggctctattccctatgcagacttggatgcgacttactatatctgaatatgaagcaaagcagttactgagcgaccccgatggactcgctaaggtcgatgagggcctctcgatggtagagcgtatcatcatgaactacattgagtctaacagttaccgcgtgactctctttgaggctctcaaacagttagtcgtagctggtaacgtcctgctgtacctaccggaaccggaagggtcaaactataatcccatgaagctgtaccgattgtcttcttatgtggtccaacgagacgcattcggcaacgttctgcaaatggtgactcgtgaccagatagcttttggtgctctccctgaggacatccgtaaggctgtagaaggtcaaggtggtgagaagaaagctgatgagacaatcgacgtgtacactcacatctatctggatgaggactcaggtgaatacctccgatacgaagaggtcgagggtatggaagtccaaggctccgatgggacttatcctaaagaggcttgcccatacatcccgattcggatggtcagactagatggtgaatcctacggtcgttcgtacattgaggaatacttaggtgacttacggtcccttgaaaatctccaagaggctatcgtcaagatgtccatgattagctctaaggttatcggcttagtgaatcctgctggtatcacccagccacgccgactgaccaaagctcagactggtgacttcgttactggtcgtccagaagacatctcgttcctccaactggagaagcaagcagactttactgtagctaaagccgtaagtgacgctatcgaggctcgcctttcgtttgcctttatgttgaactctgcggttcagcgtacaggtgaacgtgtgaccgccgaagagattcggtatgtagcttctgaacttgaagatactttaggtggtgtctactctatcctttctcaagaattacaattgcctctggtacgagtgctcttgaagcaactacaagccacgcaacagattcctgagttacctaaggaagccgtagagccaaccattagtacaggtctggaagcaattggtcgaggacaagaccttgataagctggagcggtgtgtcactgcgtgggctgcactggcacctatgcgggacgaccctgatattaaccttgcgatgattaagttacgtattgccaacgctatcggtattgacacttctggtattctactcaccgaagaacagaagcaacagaagatggcccaacagtctatgcaaatgggtatggataatggtgctgctgcgctggctcaaggtatggctgcacaagctacagcttcacctgaggctatggctgctgccgctgattccgtaggtttacagccgggaatttaatacgactcactatagggagacctcatctttgaaatgagcgatgacaagaggttggagtcctcggtcttcctgtagttcaactttaaggagacaataataatggctgaatctaatgcagacgtatatgcatcttttggcgtgaactccgctgtgatgtctggtggttccgttgaggaacatgagcagaacatgctggctcttgatgttgctgcccgtgatggcgatgatgcaatcgagttagcgtcagacgaagtggaaacagaacgtgacctgtatgacaactctgacccgttcggtcaagaggatgacgaaggccgcattcaggttcgtatcggtgatggctctgagccgaccgatgtggacactggagaagaaggcgttgagggcaccgaaggttccgaagagtttaccccactgggcgagactccagaagaactggtagctgcctctgagcaacttggtgagcacgaagagggcttccaagagatgattaacattgctgctgagcgtggcatgagtgtcgagaccattgaggctatccagcgtgagtacgaggagaacgaagagttgtccgccgagtcctacgctaagctggctgaaattggctacacgaaggctttcattgactcgtatatccgtggtcaagaagctctggtggagcagtacgtaaacagtgtcattgagtacgctggtggtcgtgaacgttttgatgcactgtataaccaccttgagacgcacaaccctgaggctgcacagtcgctggataatgcgttgaccaatcgtgacttagcgaccgttaaggctatcatcaacttggctggtgagtctcgcgctaaggcgttcggtcgtaagccaactcgtagtgtgactaatcgtgctattccggctaaacctcaggctaccaagcgtgaaggctttgcggaccgtagcgagatgattaaagctatgagtgaccctcggtatcgcacagatgccaactatcgtcgtcaagtcgaacagaaagtaatcgattcgaacttctgatagacttcgaaattaatacgactcactatagggagaccacaacggtttccctctagaaataattttgtttaactttaagaaggagatatacatatggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagctgctggtgcagtggttttcaaagtggagtaatgctgggggtggcctcaacggtcgctgctagtcccgaagaggcgagtgttacttcaacagaagaaaccttaacgccagcacaggaggccgcacgcacccgcgctgctaacaaagcccgaaaggaagctgagttggctgctgccaccgctgagcaataactagcataaccccttggggcctctaaacgggtcttgaggggttttttgctgaaaggaggaactatatgcgctcatacgatatgaacgttgagactgccgctgagttatcagctgtgaacgacattctggcgtctatcggtgaacctccggtatcaacgctggaaggtgacgctaacgcagatgcagcgaacgctcggcgtattctcaacaagattaaccgacagattcaatctcgtggatggacgttcaacattgaggaaggcataacgctactacctgatgtttactccaacctgattgtatacagtgacgactatttatccctaatgtctacttccggtcaatccatctacgttaaccgaggtggctatgtgtatgaccgaacgagtcaatcagaccgctttgactctggtattactgtgaacattattcgtctccgcgactacgatgagatgcctgagtgcttccgttactggattgtcaccaaggcttcccgtcagttcaacaaccgattctttggggcaccggaagtagagggtgtactccaagaagaggaagatgaggctagacgtctctgcatggagtatgagatggactacggtgggtacaatatgctggatggagatgcgttcacttctggtctactgactcgctaacattaataaataaggaggctctaatggcactcattagccaatcaatcaagaacttgaagggtggtatcagccaacagcctgacatccttcgttatccagaccaagggtcacgccaagttaacggttggtcttcggagaccgagggcctccaaaagcgtccacctcttgttttcttaaatacacttggagacaacggtgcgttaggtcaagctccgtacatccacctgattaaccgagatgagcacgaacagtattacgctgtgttcactggtagcggaatccgagtgttcgacctttctggtaacgagaagcaagttaggtatcctaacggttccaactacatcaagaccgctaatccacgtaacgacctgcgaatggttactgtagcagactatacgttcatcgttaaccgtaacgttgttgcacagaagaacacaaagtctgtcaacttaccgaattacaaccctaatcaagacggattgattaacgttcgtggtggtcagtatggtagggaactaattgtacacattaacggtaaagacgttgcgaagtataagataccagatggtagtcaacctgaacacgtaaacaatacggatgcccaatggttagctgaagagttagccaagcagatgcgcactaacttgtctgattggactgtaaatgtagggcaagggttcatccatgtgaccgcacctagtggtcaacagattgactccttcacgactaaagatggctacgcagaccagttgattaaccctgtgacccactacgctcagtcgttctctaagctgccacctaatgctcctaacggctacatggtgaaaatcgtaggggacgcctctaagtctgccgaccagtattacgttcggtatgacgctgagcggaaagtttggactgagactttaggttggaacactgaggaccaagttctatgggaaaccatgccacacgctcttgtgcgagccgctgacggtaatttcgacttcaagtggcttgagtggtctcctaagtcttgtggtgacgttgacaccaacccttggccttcttttgttggttcaagtattaacgatgtgttcttcttccgtaaccgcttaggattccttagtggggagaacatcatattgagtcgtacagccaaatacttcaacttctaccctgcgtccattgcgaaccttagtgatgacgaccctatagacgtagctgtgagtaccaaccgaatagcaatccttaagtacgccgttccgttctcagaagagttactcatctggtccgatgaagcacaattcgtcctgactgcctcgggtactctcacatctaagtcggttgagttgaacctaacgacccagtttgacgtacaggaccgagcgagaccttttgggattgggcgtaatgtctactttgctagtccgaggtccagcttcacgtccatccacaggtactacgctgtgcaggatgtcagttccgttaagaatgctgaggacattacatcacacgttcctaactacatccctaatggtgtgttcagtatttgcggaagtggtacggaaaacttctgttcggtactatctcacggggaccctagtaaaatcttcatgtacaaattcctgtacctgaacgaagagttaaggcaacagtcgtggtctcattgggactttggggaaaacgtacaggttctagcttgtcagagtatcagctcagatatgtatgtgattcttcgcaatgagttcaatacgttcctagctagaatctctttcactaagaacgccattgacttacagggagaaccctatcgtgcctttatggacatgaagattcgatacacgattcctagtggaacatacaacgatgacacattcactacctctattcatattccaacaatttatggtgcaaacttcgggaggggcaaaatcactgtattggagcctgatggtaagataaccgtgtttgagcaacctacggctgggtggaatagcgacccttggctgagactcagcggtaacttggagggacgcatggtgtacattgggttcaacattaacttcgtatatgagttctctaagttcctcatcaagcagactgccgacgacgggtctacctccacggaagacattgggcgcttacagttacgccgagcgtgggttaactacgagaactctggtacgtttgacatttatgttgagaaccaatcgtctaactggaagtacacaatggctggtgcccgattaggctctaacactctgagggctgggagactgaacttagggaccggacaatatcgattccctgtggttggtaacgccaagttcaacactgtatacatcttgtcagatgagactacccctctgaacatcattgggtgtggctgggaaggtaactacttacggagaagttccggtatttaattaaatattctccctgtggtggctcgaaattaatacgactcactatagggagaacaatacgactacgggagggttttcttatgatgactataagacctactaaaagtacagactttgaggtattcactccggctcaccatgacattcttgaagctaaggctgctggtattgagccgagtttccctgatgcttccgagtgtgtcacgttgagcctctatgggttccctctagctatcggtggtaactgcggggaccagtgctggttcgttacgagcgaccaagtgtggcgacttagtggaaaggctaagcgaaagttccgtaagttaatcatggagtatcgcgataagatgcttgagaagtatgatactctttggaattacgtatgggtaggcaatacgtcccacattcgtttcctcaagactatcggtgcggtattccatgaagagtacacacgagatggtcaatttcagttatttacaatcacgaaaggaggataaccatatgtgttgggcagccgcaatacctatcgctatatctggcgctcaggctatcagtggtcagaacgctcaggccaaaatgattgccgctcagaccgctgctggtcgtcgtcaagctatggaaatcatgaggcagacgaacatccagaatgctgacctatcgttgcaagctcgaagtaaacttgaggaagcgtccgccgagttgacctcacagaacatgcagaaggtccaagctattgggtctatccgagcggctatcggagagagtatgcttgaaggttcctcaatggaccgcattaagcgagtcacagaaggacagttcattcgggaagccaatatggtaactgagaactatcgccgtgactaccaagcaatcttcgcacagcaacttggtggtactcaaagtgctgcaagtcagattgacgaaatctataagagcgaacagaaacagaagagtaagctacagatggttctggacccactggctatcatggggtcttccgctgcgagtgcttacgcatccggtgcgttcgactctaagtccacaactaaggcacctattgttgccgctaaaggaaccaagacggggaggtaatgagctatgagtaaaattgaatctgcccttcaagcggcacaaccgggactctctcggttacgtggtggtgctggaggtatgggctatcgtgcagcaaccactcaggccgaacagccaaggtcaagcctattggacaccattggtcggttcgctaaggctggtgccgatatgtataccgctaaggaacaacgagcacgagacctagctgatgaacgctctaacgagattatccgtaagctgacccctgagcaacgtcgagaagctctcaacaacgggacccttctgtatcaggatgacccatacgctatggaagcactccgagtcaagactggtcgtaacgctgcgtatcttgtggacgatgacgttatgcagaagataaaagagggtgtcttccgtactcgcgaagagatggaagagtatcgccatagtcgccttcaagagggcgctaaggtatacgctgagcagttcggcatcgaccctgaggacgttgattatcagcgtggtttcaacggggacattaccgagcgtaacatctcgctgtatggtgcgcatgataacttcttgagccagcaagctcagaagggcgctatcatgaacagccgagtggaactcaacggtgtccttcaagaccctgatatgctgcgtcgtccagactctgctgacttctttgagaagtatatcgacaacggtctggttactggcgcaatcccatctgatgctcaagccacacagcttataagccaagcgttcagtgacgcttctagccgtgctggtggtgctgacttcctgatgcgagtcggtgacaagaaggtaacacttaacggagccactacgacttaccgagagttgattggtgaggaacagtggaacgctctcatggtcacagcacaacgttctcagtttgagactgacgcgaagctgaacgagcagtatcgcttgaagattaactctgcgctgaaccaagaggacccaaggacagcttgggagatgcttcaaggtatcaaggctgaactagataaggtccaacctgatgagcagatgacaccacaacgtgagtggctaatctccgcacaggaacaagttcagaatcagatgaacgcatggacgaaagctcaggccaaggctctggacgattccatgaagtcaatgaacaaacttgacgtaatcgacaagcaattccagaagcgaatcaacggtgagtgggtctcaacggattttaaggatatgccagtcaacgagaacactggtgagttcaagcatagcgatatggttaactacgccaataagaagctcgctgagattgacagtatggacattccagacggtgccaaggatgctatgaagttgaagtaccttcaagcggactctaaggacggagcattccgtacagccatcggaaccatggtcactgacgctggtcaagagtggtctgccgctgtgattaacggtaagttaccagaacgaaccccagctatggatgctctgcgcagaatccgcaatgctgaccctcagttgattgctgcgctatacccagaccaagctgagctattcctgacgatggacatgatggacaagcagggtattgaccctcaggttattcttgatgccgaccgactgactgttaagcggtccaaagagcaacgctttgaggatgataaagcattcgagtctgcactgaatgcatctaaggctcctgagattgcccgtatgccagcgtcactgcgcgaatctgcacgtaagatttatgactccgttaagtatcgctcggggaacgaaagcatggctatggagcagatgaccaagttccttaaggaatctacctacacgttcactggtgatgatgttgacggtgataccgttggtgtgattcctaagaatatgatgcaggttaactctgacccgaaatcatgggagcaaggtcgggatattctggaggaagcacgtaagggaatcattgcgagcaacccttggataaccaataagcaactgaccatgtattctcaaggtgactccatttaccttatggacaccacaggtcaagtcagagtccgatacgacaaagagttactctcgaaggtctggagtgagaaccagaagaaactcgaagagaaagctcgtgagaaggctctggctgatgtgaacaagcgagcacctatagttgccgctacgaaggcccgtgaagctgctgctaaacgagtccgagagaaacgtaaacagactcctaagttcatctacggacgtaaggagtaactaaaggctacataaggaggccctaaatggataagtacgataagaacgtaccaagtgattatgatggtctgttccaaaaggctgctgatgccaacggggtctcttatgaccttttacgtaaagtcgcttggacagaatcacgatttgtgcctacagcaaaatctaagactggaccattaggcatgatgcaatttaccaaggcaaccgctaaggccctcggtctgcgagttaccgatggtccagacgacgaccgactgaaccctgagttagctattaatgctgccgctaagcaacttgcaggtctggtagggaagtttgatggcgatgaactcaaagctgcccttgcgtacaaccaaggcgagggacgcttgggtaatccacaacttgaggcgtactctaagggagacttcgcatcaatctctgaggagggacgtaactacatgcgtaaccttctggatgttgctaagtcacctatggctggacagttggaaacttttggtggcataaccccaaagggtaaaggcattccggctgaggtaggattggctggaattggtcacaagcagaaagtaacacaggaacttcctgagtccacaagttttgacgttaagggtatcgaacaggaggctacggcgaaaccattcgccaaggacttttgggagacccacggagaaacacttgacgagtacaacagtcgttcaaccttcttcggattcaaaaatgctgccgaagctgaactctccaactcagtcgctgggatggctttccgtgctggtcgtctcgataatggttttgatgtgtttaaagacaccattacgccgactcgctggaactctcacatctggactccagaggagttagagaagattcgaacagaggttaagaaccctgcgtacatcaacgttgtaactggtggttcccctgagaacctcgatgacctcattaaattggctaacgagaactttgagaatgactcccgcgctgccgaggctggcctaggtgccaaactgagtgctggtattattggtgctggtgtggacccgcttagctatgttcctatggtcggtgtcactggtaagggctttaagttaatcaataaggctcttgtagttggtgccgaaagtgctgctctgaacgttgcatccgaaggtctccgtacctccgtagctggtggtgacgcagactatgcgggtgctgccttaggtggctttgtgtttggcgcaggcatgtctgcaatcagtgacgctgtagctgctggactgaaacgcagtaaaccagaagctgagttcgacaatgagttcatcggtcctatgatgcgattggaagcccgtgagacagcacgaaacgccaactctgcggacctctctcggatgaacactgagaacatgaagtttgaaggtgaacataatggtgtcccttatgaggacttaccaacagagagaggtgccgtggtgttacatgatggctccgttctaagtgcaagcaacccaatcaaccctaagactctaaaagagttctccgaggttgaccctgagaaggctgcgcgaggaatcaaactggctgggttcaccgagattggcttgaagaccttggggtctgacgatgctgacatccgtagagtggctatcgacctcgttcgctctcctactggtatgcagtctggtgcctcaggtaagttcggtgcaacagcttctgacatccatgagagacttcatggtactgaccagcgtacttataatgacttgtacaaagcaatgtctgacgctatgaaagaccctgagttctctactggcggcgctaagatgtcccgtgaagaaactcgatacactatctaccgtagagcggcactagctattgagcgtccagaactacagaaggcactcactccgtctgagagaatcgttatggacatcattaagcgtcactttgacaccaagcgtgaacttatggaaaacccagcaatattcggtaacacaaaggctgtgagtatcttccctgagagtcgccacaaaggtacttacgttcctcacgtatatgaccgtcatgccaaggcgctgatgattcaacgctacggtgccgaaggtttgcaggaagggattgcccgctcatggatgaacagctacgtctccagacctgaggtcaaggccagagtcgatgagatgcttaaggaattacacggggtgaaggaagt

SEQ ID NO: 2 - DNA, 836 bp, synthetic construct
  source 1..836: mol_type = other DNA
tttaagaaggagatatacatatgaaatacttattgccgaccgccgccgctggtttgcttttgttagcagcccagcctgcaatggccatggctctgcccaaaacgggcaagccgactgctaagcaggtggttgattgggctatcaatcttatcggctctggcgtagatgtggatgggtactatgggcgccaatgttgggatttgccgaattacatttttaaccgttattggaattttaagactcctgggaatgctcgtgatatggcctggtatcgctatccggagggatttaaagtttttcgtaacacttctgatttcgtcccaaaacccggcgatattgcggtatggaccggtgggaactataattggaatacttgggggcatactggcattgtagttggaccgtcgacaaaatcctatttttactcggttgaccaaaactggaacaattcaaattcgtatgtagggtcgccagcggcaaaaattaaacattcttatttcggtgtgactcatttcgtccgtccagcatacaaggcggagccgaagcctacaccacctagtcgctcccgcagctatcgcgaaacaggcacaatgactgtcacagtggacgcccttaatgtgcgccgtgcgccgaacactagcggagaaatcgttgccgtctacaagcgcggggagtccttcgattacgacacagtcattatcgacgtaaatgggtacgtgtgggtgtcgtacattggaggtagcggaaagcgcaattacgtggctaccggagcgacaaaggacgggaagcgctttggaaacgcgtggggaacttttaaataagtcgaccggctgcta

SEQ ID NO: 3 - DNA, 21 bp, synthetic construct
  source 1..21: mol_type = other DNA
gtcgaccggctgctaacaaag

SEQ ID NO: 4 - DNA, 43 bp, synthetic construct
  source 1..43: mol_type = other DNA
atgtatatctccttcttaaagttaaacaaaattatttctagag

SEQ ID NO: 5 - DNA, 22 bp, synthetic construct
  source 1..22: mol_type = other DNA
tatagtcctgtcgggtttcgcc

SEQ ID NO: 6 - DNA, 22 bp, synthetic construct
  source 1..22: mol_type = other DNA
ctggcctgttgaacaagtctgg

SEQ ID NO: 7 - DNA, 37 bp, synthetic construct
  source 1..37: mol_type = other DNA
accgctgagcaataataatacgactcactatagggag

SEQ ID NO: 8 - DNA, 34 bp, synthetic construct
  source 1..34: mol_type = other DNA
aaggggttatgctaggcaaaaaacccctcaagac

SEQ ID NO: 9 - DNA, 30 bp, synthetic construct
  source 1..30: mol_type = other DNA
tctcacagtgtacggacctaaagttccccc

SEQ ID NO: 10 - DNA, 30 bp, synthetic construct
  source 1..30: mol_type = other DNA
attacgcgatgacagtagacaacctttccg

SEQ ID NO: 11 - DNA, 30 bp, synthetic construct
  source 1..30: mol_type = other DNA
tgcagcaataccggaaaggttgtctactgt

SEQ ID NO: 12 - DNA, 30 bp, synthetic construct
  source 1..30: mol_type = other DNA
atatgtctcctcatagatgtgcctatgtgg

SEQ ID NO: 13 - DNA, 30 bp, synthetic construct
  source 1..30: mol_type = other DNA
acttgtgactccacataggcacatctatga

SEQ ID NO: 14 - DNA, 30 bp, synthetic construct
  source 1..30: mol_type = other DNA
gaataacctgagggtcaataccctgcttgt

SEQ ID NO: 15 - DNA, 30 bp, synthetic construct
  source 1..30: mol_type = other DNA
gacatgatggacaagcagggtattgaccct

SEQ ID NO: 16 - DNA, 30 bp, synthetic construct
  source 1..30: mol_type = other DNA
agggacacagagagacactcaaggtaacac

SEQ ID NO: 17 - DNA, 27 bp, synthetic construct
  source 1..27: mol_type = other DNA
acttgtgactccacataggcacatcta

SEQ ID NO: 18 - DNA, 33 bp, synthetic construct
  source 1..33: mol_type = other DNA
tagtgagtcgtattattattgctcagcggtggc

SEQ ID NO: 19 - DNA, 33 bp, synthetic construct
  source 1..33: mol_type = other DNA
tgaggggttttttgcctagcataaccccttggg

SEQ ID NO: 20 - DNA, 30 bp, synthetic construct; identical to SEQ ID NO: 14
  source 1..30: mol_type = other DNA

SEQ ID NO: 21 - DNA, 22 bp, synthetic construct
  source 1..22: mol_type = other DNA
gaaaccttaacgccagcacagg

SEQ ID NO: 22 - DNA, 22 bp, synthetic construct
  source 1..22: mol_type = other DNA
accgatagacgccagaatgtcg

SEQ ID NO: 23 - DNA, 916 bp, synthetic construct
  source 1..916: mol_type = other DNA
taatacgactcactatagggagaccacaacggtttccctctagaaataattttgtttaactttaagaaggagatatacatatgagcaaaggtgaagaactgtttaccggcgttgtgccgattctggtggaactggatggcgatgtgaacggtcacaaattcagcgtgcgtggtgaaggtgaaggcgatgccacgattggcaaactgacgctgaaatttatctgcaccaccggcaaactgccggtgccgtggccgacgctggtgaccaccctgacctatggcgttcagtgttttagtcgctatccggatcacatgaaacgtcacgatttctttaaatctgcaatgccggaaggctatgtgcaggaacgtacgattagctttaaagatgatggcaaatataaaacgcgcgccgttgtgaaatttgaaggcgataccctggtgaaccgcattgaactgaaaggcacggattttaaagaagatggcaatatcctgggccataaactggaatacaactttaatagccataatgtttatattacggcggataaacagaaaaatggcatcaaagcgaattttaccgttcgccataacgttgaagatggcagtgtgcagctggcagatcattatcagcagaataccccgattggtgatggtccggtgctgctgccggataatcattatctgagcacgcagaccgttctgtctaaagatccgaacgaaaaaggcacgcgggaccacatggttctgcacgaatatgtgaatgcggcaggtattacgtggagccatccgcagttcgaaaaataagtcgaccggctgctaacaaagcccgaaaggaagctgagttggctgctgccaccgctgagcaataactagcataaccccttggggcctctaaacgggtcttgaggggttttttg

SEQ ID NO: 24 - DNA, 21 bp, synthetic construct
  source 1..21: mol_type = other DNA
cgctcggctatttcacatact

SEQ ID NO: 25 - DNA, 22 bp, synthetic construct
  source 1..22: mol_type = other DNA
ggtttctggcgttaaaccaatc

SEQ ID NO: 26 - DNA, 22 bp, synthetic construct
  source 1..22: mol_type = other DNA
ccgtctggtttctgacgatatt

SEQ ID NO: 27 - DNA, 20 bp, synthetic construct
  source 1..20: mol_type = other DNA
cgcagcaggatctggttaat

SEQ ID NO: 28 - DNA, 21 bp, synthetic construct
  source 1..21: mol_type = other DNA
gtgcgcctcttctttgaattt

SEQ ID NO: 29 - DNA, 19 bp, synthetic construct
  source 1..19: mol_type = other DNA
tcgatggtgcgtccattac

SEQ ID NO: 30 - DNA, 21 bp, synthetic construct
  source 1..21: mol_type = other DNA
aatatggcatccgtggtatcc

SEQ ID NO: 31 - DNA, 21 bp, synthetic construct
  source 1..21: mol_type = other DNA
gttagcgtcgaggaactcttt

SEQ ID NO: 32 - DNA, 22 bp, synthetic construct
  source 1..22: mol_type = other DNA
cctgttcgatgagaggaatctg

SEQ ID NO: 33 - DNA, 21 bp, synthetic construct
  source 1..21: mol_type = other DNA
ccttagctgggtgagtgattt

SEQ ID NO: 34 - DNA, 6458 bp, synthetic construct
  source 1..6458: mol_type = other DNA
ggctcaccttcgggtgggcctttctgcgtttatacagagacctcagccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcctccagcttttgttccctttagtgagggttaattgcgcgcttggcgtaatcatggtcatagctgtttcctgtgtgaaaggtctcaatctacaacagtagaaattacggtgtttactagtattatacctaggactgagctagctgtcaaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgttactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtccaccacaattcagcaaattgtgaacatcatcacgttcatctttccctggttgccaatggcccattttcctgtcagtaacgagaaggtcgcgaattcaggcgctttttagactggtcgtaatgaaattctttttaagaaggagatatacattaaagaggagaaaggatctatgagcatctaccaggagttcgtcaacaagtattcactgagtaagacactgcggttcgagctgatcccacagggcaagacactggagaacatcaaggcccgaggcctgattctggacgatgagaagcgggcaaaagactataagaaagccaagcagatcattgataaataccaccagttctttatcgaggaaattctgagctccgtgtgcatcagtgaggatctgctgcagaattactcagacgtgtacttcaagctgaagaagagcgacgatgacaacctgcagaaggacttcaagtccgccaaggacaccatcaagaaaagtacacgtctgagtaattccagattagcgagtacatcaaggactccgaaaagtttaaaaatctgttcaaccagaatctgatcgatgctaagaaaggccaggagtccgacctgatcctgtggctgaaacagtctaaggacaatgggattgaactgttcaaggctaactccgatatcactgatattgacgaggcactggaaatcatcaagagcttcaagggatggaccacatactttaaaggcttccacgagaaccgcaagaacgtgtactccagcaacgacattcctacctccatcatctaccgaatcgtcgatgacaatctgccaaagttcctggagaacaaggccaaatatgaatctctgaaggacaaagctcccgaggcaattaattacgaacagatcaagaaagatctggctgaggaactgacattcgatatcgactataagactagcgaggtgaaccagagggtcttttccctggacgaggtgtttgaaatcgccaatttcaacaattacctgaaccagtccggcattactaaattcaataccatcattggcgggaagtttgtgaacggggagaataccaagcgcaagggaattaacgaatacatcaatctgtatagccagcagatcaacgacaaaactctgaagaaatacaagatgtctgtgctgttcaaacagatcctgagtgataccgagtccaagtcttttgtcattgataaactggaagatgactcagacgtggtcactaccatgcagagcttttatgagcagatcgccgctttcaagacagtggaggaaaaatctattaaggaaactctgagtctgctgttcgatgacctgaaagcccagaagctggacctgagtaagatctacttcaaaaacgataagagtctgacagacctgtcacagcaggtgtttgatgactattccgtgattgggaccgccgtcctggagtacattacacagcagatcgctccaaagaacctggataatccctctaagaaagagcaggaactgatcgctaagaaaaccgagaaggcaaaatatctgagtctggaaacaattaagctggcactggaggagttcaacaagcacagggatattgacaaacagtgccgctttgaggaaatcctggccaacttcgcagccatccccatgatttttgatgagatcgcccagaacaaagacaatctggctcagatcagtattaagtaccagaaccagggcaagaaagacctgctgcaggcttcagcagaagatgacgtgaaagccatcaaggatctgctggaccagaccaacaatctgctgcacaagctgaaaatcttccatattagtcagtcagaggataaggctaatatcctggataaagacgaacacttctacctggtgttcgaggaatgttacttcgagctggcaaacattgtccccctgtataacaagattaggaactacatcacacagaagccttactctgacgagaagtttaaactgaacttcgaaaatagtaccctggccaacgggtgggataagaacaaggagcctgacaacacagctatcctgttcatcaaggatgacaagtactatctgggagtgatgaataagaaaaacaataagatcttcgatgacaaagccattaaggagaacaaaggggaaggatacaagaaaatcgtgtataagctgctgcccggcgcaaataagatgctgcctaaggtgttcttcagcgccaagagtatcaaattctacaacccatccgaggacatcctgcggattagaaatcactcaacacatactaagaacgggagcccccagaagggatatgagaaatttgagttcaacatcgaggattgcaggaagtttattgacttctacaagcagagcatctccaaacaccctgaatggaaggattttggcttccggttttccgacacacagagatataactctatcgacgagttctaccgcgaggtggaaaatcaggggtataagctgacttttgagaacatttctgaaagttacatcgacagcgtggtcaatcagggaaagctgtacctgttccagatctataacaaagatttttcagcatacagcaagggcagaccaaacctgcatacactgtactggaaggccctgttcgatgagaggaatctgcaggacgtggtctataaactgaacggagaggccgaactgttttaccggaagcagtctattcctaagaaaatcactcacccagctaaggaggccatcgctaacaagaacaaggacaatcctaagaaagagagcgtgttcgaatacgatctgattaaggacaagcggttcaccgaagataagttctttttccattgtccaatcaccattaacttcaagtcaagcggcgctaacaagttcaacgacgagatcaatctgctgctgaaggaaaaagcaaacgatgtgcacatcctgagcattgctcgaggagagcggcatctggcctactataccctggtggatggcaaagggaatatcattaagcaggatacattcaacatcattggcaatgaccggatgaaaaccaactaccacgataaactggctgcaatcgagaaggatagagactcagctaggaaggactggaagaaaatcaacaacattaaggagatgaaggaaggctatctgagccaggtggtccatgagattgcaaagctggtcatcgaatacaatgccattgtggtgttcgaggatctgaacttcggctttaagagggggcgctttaaggtggaaaaacaggtctatcagaagctggagaaaatgctgatcgaaaagctgaattacctggtgtttaaagataacgagttcgacaagaccggaggcgtcctgagagcctaccagctgacagctccctttgaaactttcaagaaaatgggaaaacagacaggcatcatctactatgtgccagccggattcacttccaagatctgccccgtgaccggctttgtcaaccagctgtaccctaaatatgagtcagtgagcaagtcccaggaatttttcagcaagttcgataagatctgttataatctggacaaggggtacttcgagttttccttcgattacaagaacttcggcgacaaggccgctaaggggaaatggaccattgcctccttcggatctcgcctgatcaactttcgaaattccgataaaaaccacaattgggacactagggaggtgtacccaaccaaggagctggaaaagctgctgaaagactactctatcgagtatggacatggcgaatgcatcaaggcagccatctgtggcgagagtgataagaaatttttcgccaagctgacctcagtgctgaatacaatcctgcagatgcggaactcaaagaccgggacagaactggactatctgattagccccgtggctgatgtcaacggaaacttcttcgacagcagacaggcacccaaaaatatgcctcaggatgcagacgccaacggggcctaccacatcgggctgaagggactgatgctgctgggccggatcaagaacaatcaggaggggaagaagctgaacctggtcattaagaacgaggaatacttcgagtttgtccagaatagaaataacaaaaggccggcggccacgaaaaaggccggccaggcaaaaaagaaaaagggatcctacccatacgatgttccagattacgcttatccctacgacgtgcctgattatgcatacccatatgatgtccccgactatgcctaacaaataaaacgaaaggctcagtcgaaagactgggcctttcgttttatctgttgtttgtcggtgaacgctctctactagagtcacact

SEQ ID NO: 35 - DNA, 2486 bp, synthetic construct
  source 1..2486: mol_type = other DNA
tccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggagaccacaacggtttccctctagaaataattttgtttaactttaagaaggagatatacatatgagcaaaggtgaagaactgtttaccggcgttgtgccgattctggtggaactggatggcgatgtgaacggtcacaaattcagcgtgcgtggtgaaggtgaaggcgatgccacgattggcaaactgacgctgaaatttatctgcaccaccggcaaactgccggtgccgtggccgacgctggtgaccaccctgacctatggcgttcagtgttttagtcgctatccggatcacatgaaacgtcacgatttctttaaatctgcaatgccggaaggctatgtgcaggaacgtacgattagctttaaagatgatggcaaatataaaacgcgcgccgttgtgaaatttgaaggcgataccctggtgaaccgcattgaactgaaaggcacggattttaaagaagatggcaatatcctgggccataaactggaatacaactttaatagccataatgtttatattacggcggataaacagaaaaatggcatcaaagcgaattttaccgttcgccataacgttgaagatggcagtgtgcagctggcagatcattatcagcagaataccccgattggtgatggtccggtgctgctgccggataatcattatctgagcacgcagaccgttctgtctaaagatccgaacgaaaaaggcacgcgggaccacatggttctgcacgaatatgtgaatgcggcaggtattacgtggagccatccgcagttcgaaaaataagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtgagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagct

SEQ ID NO: 36 - DNA, 5429 bp, synthetic construct
  source 1..5429: mol_type = other DNA
ttatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatactacctgacgctttttatcgcaactctctactgtttctccatacccgtttttttgggctagaaataattttgtttaactttaagaaggagatatacatatgcggggttctcatcatcatcatcatcatggtatggctagcatgactggtggacagcaaatgggtcgggatctgtacgagaacctgtacttccagggctcgagcatggtgagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcgccacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccaacttcaagatccgccacaacgtggaggacggcagcgtgcagctcgccgaccactaccagcagaacacccccatcggcgacggccccgtgctgctgcccgacaaccactacctgagcacccagtccgtgctgagcaaagaccccaacgagaagcgcgatcacatggtcctgctggagttcgtgaccgccgccgggatcactcacggcatggacgagctgtacaagtaagaattcgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatctggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaa

SEQ ID NO: 37 - DNA, 330 bp, synthetic construct
  source 1..330: mol_type = other DNA
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcgtaa

SEQ ID NO: 38 - DNA, 966 bp, synthetic construct
  source 1..966: mol_type = other DNA
atgggcacgaccaaacacagtaaactgcttatcctgggttcaggcccggcgggatacaccgctgctgtctacgcggcgcgcgccaacctgcaacctgtgctgattaccggcatggaaaaaggcggccaactgaccaccaccacggaagtggaaaactggcctggcgatccaaacgatctgaccggtccgttattaatggagcgcatgcacgaacatgccaccaagtttgaaactgagatcatttttgatcatatcaacaaggtggatctgcaaaaccgtccgttccgtctgaatggcgataacggcgaatacacttgcgacgcgctgattattgccaccggagcttctgcacgctatctcggcctgccctctgaagaagcctttaaaggccgtggggtttctgcttgtgcaacctgcgacggtttcttctatcgcaaccagaaagttgcggtcatcggcggcggcaataccgcggttgaagaggcgctgtatctgtctaacatcgcttcggaagtgcatctgattcaccgccgtgacggtttccgcgcggaaaaaatcctcattaagcgcctgatggataaagtggagaacggcaacatcattctgcacaccaaccgtacgctggaagaagtgaccggcgatcaaatgggtgtcactggcgttcgtctgcgcgatacgcaaaacagcgataacatcgagtcactcgacgttgccggtctgtttgttgctatcggtcacagcccgaatactgcgattttcgaagggcagctggaactggaaaacggctacatcaaagtacagtcgggtattcatggtaatgccacccagaccagcattcctggcgtctttgccgcaggcgacgtgatggatcacatttatcgccaggccattacttcggccggtacaggctgcatggcagcacttgatgcggaacgctacctcgatggtttagctgacgcaaaataa

SEQ ID NO: 39 - DNA, 543 bp, synthetic construct
  source 1..543: mol_type = other DNA
attaaaggcggaaaacgagttcaaacggcgcgccctaaccgtatcaatggcgaaattcgcgcccaggaagttcgcttaacaggtctggaaggcgagcagcttggtattgtgagtctgagagaagctctggagaaagcagaagaagccggagtagacttagtcgagatcagccctaacgccgagccgccggtttgtcgtataatggattacggcaaattcctctatgaaaagagcaagtcttctaaggaacagaagaaaaagcaaaaagttatccaggttaaggaaattaaattccgtcctggtacagatgaaggcgactatcaggtaaaactccgcagcctgattcgctttctcgaagagggtgataaagccaaaatcacgctgcgtttccgcggtcgtgagatggcgcaccagcaaatcggtatggaagtgcttaatcgcgtgaaagacgatttgcaagaactggcagtggtcgaatccttcccaacgaagatcgaaggccgccagatgatcatggtgctcgctcctaagaagaaacagtaa

SEQ ID NO: 40 - DNA, 1518 bp, synthetic construct
  source 1..1518: mol_type = other DNA
atggcacagattgatttccgaaaaaaaataaactggcatcgtcgttaccgttcaccgcagggcgttaaaaccgaacatgagatcctgcggatcttcgagagcgatcgcgggcgtatcatcaactctccggcaattcgtcgtctgcaacaaaagacccaggtttttccactggagcgcaatgccgccgtgcgcacgcgtcttacccactcgatggaagtccagcaggtgggacgctacatcgccaaagaaattttaagccgtctgaaagagcttaaattgctggaagcatacggcctggatgaactgaccggcccctttgaaagcattgttgagatgtcatgcctgatgcacgatatcggcaatccgccgtttggtcattttggcgaagcagcgataaatgactggtttcgccaacgtttgcacccggaagatgccgaaagccagcctctgactgacgatcgctgcagcgtggcggcactacgtttacgggacggggaagaaccgcttaacgagctgcggcgcaagattcgtcaggacttatgtcattttgaggggaatgcacaaggcattcgtctggtgcatacattgatgcggatgaatctcacctgggcacaggttggcggtattttaaaatatacccgtccggcgtggtggcgtggcgaaacgcctgagacacatcactatttaatgaaaaagccgggttattatctttctgaagaagcctatattgcgcggttgcgtaaagaacttaatttggcgctttacagtcgttttccattaacgtggattatggaagctgccgacgacatctcctattgtgtggcagaccttgaagatgcggtagagaaaagaatatttaccgttgagcagctttatcatcatttgcacgaagcgtggggccagcatgagaaaggttcgctcttttcgctagtggttgaaaatgcctgggaaaaatcacgctcaaatagtttaagccgcagtacggaagatcagttttttatgtatttacgagtaaacaccctaaataaactggtaccctacgcggcacaacgatttattgataatctgcctgcgattttcgccggaacgtttaatcatgcattattggaagatgccagcgaatgcagcgatcttcttaagctatataaaaatgtcgctgtaaaacatgtgtttagccatccagatgtcgagcagcttgaattgcagggctatcgggtcattagcggattattagagatttatcgtcctttattaagcctgtcgttatcagactttactgaactggtagaaaaagaacgggtgaaacgtttccctattgaatcgcgcttattccacaaactctcgacgcgccatcggctggcctatgtcgaggctgtcagtaaattaccgtcagattctcctgagtttccgctatgggaatattattaccgttgccgcctgctgcaggattatatcagcggtatgaccgacctctatgcgtgggatgaataccgacgtctgatggccgtagaacaataa

SEQ ID NO: 41 - DNA, 150 bp, synthetic construct
  source 1..150: mol_type = other DNA
attctgactgataattgctcacagaaacggagcggcacctcttttaacccttgaagtcactgcccgtttcgagagtttctcaactcgaataactaaagccaacgtgaacttttgcggatctccaggatccgcttttttttgccataaaaa

SEQ ID NO: 42 - DNA, 87 bp, synthetic construct
  source 1..87: mol_type = other DNA
gctgaaaaacataacccataaaatgctagctgtaccaggaaccacctccttagcctgtgtaatctcccttacacgggcttatttttt

SEQ ID NO: 43 - DNA, 723 bp, synthetic construct
  source 1..723: mol_type = other DNA
atgagcaaaggtgaagaactgtttaccggcgttgtgccgattctggtggaactggatggcgatgtgaacggtcacaaattcagcgtgcgtggtgaaggtgaaggcgatgccacgattggcaaactgacgctgaaatttatctgcaccaccggcaaactgccggtgccgtggccgacgctggtgaccaccctgacctatggcgttcagtgttttagtcgctatccggatcacatgaaacgtcacgatttctttaaatctgcaatgccggaaggctatgtgcaggaacgtacgattagctttaaagatgatggcaaatataaaacgcgcgccgttgtgaaatttgaaggcgataccctggtgaaccgcattgaactgaaaggcacggattttaaagaagatggcaatatcctgggccataaactggaatacaactttaatagccataatgtttatattacggcggataaacagaaaaatggcatcaaagcgaattttaccgttcgccataacgttgaagatggcagtgtgcagctggcagatcattatcagcagaataccccgattggtgatggtccggtgctgctgccggataatcattatctgagcacgcagaccgttctgtctaaagatccgaacgaaaaaggcacgcgggaccacatggttctgcacgaatatgtgaatgcggcaggtattacgtggagccatccgcagttcgaaaaataa

SEQ ID NO: 44 - DNA, 4461 bp, synthetic construct
  source 1..4461: mol_type = other DNA
atgattgaacgcggtaaatttcgctcactgacgctgattaactggaacggcttttttgcccgaacttttgaccttgacgagctggtcacgacgctttctggcggtaacggggcgggtaaatccaccaccatggcggcgttcgttacggcgctgatccccgacctgaccctgttgcatttccgtaacactacggaagccggggccaccagcggttcgcgcgataaaggtctgcacggtaagctgaaagcgggtgtctgttattcgatgctcgacaccattaactcgcgccaccagcgcgtggtggtcggtgtgcgtctgcaacaggttgccggacgcgatcgtaaagtggatatcaaaccatttgccattcagggactgccgatgtcggtgcagccgacacagctggtgaccgaaaccctgaacgaacgccaggcgcgcgtgctgccgcttaacgagctgaaagacaagctcgaggcgatggaaggcgtgcagtttaaacagttcaactccattaccgattaccactcgctgatgttcgatctgggcatcatcgcgcgtcgtctgcgctccgcatctgaccgtagcaaattctatcgtctgatcgaagcttcgctgtatggcgggatctccagtgccattacccgttctctgcgcgactacctgttgccggaaaacagcggcgtgcgtaaagcgttccaggacatggaagcggcgctgcgtgaaaaccgcatgacgctggaagcgattcgtgtcacccagtcggaccgtgacctgtttaagcatctgatcagcgaagctaccaactacgtggcggcggactacatgcgtcacgccaacgagcgccgtgtccatctcgacaaagccctggagtttcgtcgcgagttacatacttcgcgtcagcaactggcggctgagcagtacaaacacgtcgatatggcgcgtgagctggcagagcacaacggtgccgaaggcgatctggaagcggattatcaggcggccagcgatcacctgaacctggtgcaaaccgcactgcgtcagcaggagaagatcgaacgctacgaagcggatctcgatgagctgcagatccgtctggaagagcaaaacgaagtggtggcagaagccatcgaacgccaggaagagaacgaggctcgcgcggaagctgccgaactggaagtggacgagctgaaaagccagcttgctgactaccagcaggcgctggacgtccagcaaacgcgcgcgatccagtataaccaggcgattgctgcacttaatcgtgccaaagaactgtgccatctgccggacttaaccgccgacagcgccgccgaatggctggaaaccttccaggcgaaagagctggaagcgactgaaaaaatgctctctcttgagcagaaaatgagcatggcgcaaaccgcgcacagccagtttgagcaggcttatcagctggtggtggcaatcaacggcccactggcgcgtaacgaggcgtgggatgtcgctcgcgaactattgcgcgaaggggtcgatcagcgtcacctggcagagcaggttcagccgctgcggatgcgattaagcgaactggaacagcgtctacgcgagcagcaagaagctgagcgtctgctggcagatttctgcaaacgtcagggcaagaattttgatatcgacgaactggaagccctgcatcaggaactggaagcacgcattgcctctctttccgatagcgtgtctaacgcccgtgaagagcgcatggcactgcgccaggagcaggaacagctgcagtctcgcattcagagtttgatgcagcgtgcgccggtttggctggcagcgcaaaacagtctcaaccagttgagcgaacagtgcggcgaagagtttacctccagccaggacgtcaccgaatatctgcaacagttgctggagcgtgagcgagaggcgattgttgaacgcgatgaagtgggcgcgcgcaaaaacgccgtcgatgaagagatcgaacgtttaagccagcctggcggctctgaagatcagcgtctgaacgcgctggcggagcgttttggtggtgtgctgctgtcagaaatttatgacgacgttagcctggaagatgcgccgtacttctcagcgctgtatggcccgtcacgccacgccatcgtggtgccagatctgtcacaggtaactgaacacctggaaggcttgaccgattgcccggaagatctctatctgatcgaaggagatccgcagtcattcgatgacagcgtgttcagcgttgatgagctggaaaaagcggtagtggtgaaaatcgccgatcgtcagtggcgttattcacgtttcccggaagtgccgctgtttggtcgtgctgcgcgtgaaagccgtattgaaagcctccatgccgagcgtgaagtgctttccgaacgcttcgccacgctctcctttgatgtacagaaaactcagcgtctgcatcaggcgttcagccgctttatcggcagtcatctggcggttgcgtttgagtctgacccggaagcagaaatccgtcaactgaacagccgtcgcgtcgaactggagcgggcgttaagtaatcatgaaaatgataaccagcagcagcgtattcagtttgagcaggcgaaagagggcgttacggcgctgaaccgcattctgccgcgtctcaacctgttggctgatgacagcctggcggatcgcgtcgatgaaatccgcgaacgtctggatgaagcccaggaagccgcgcgttttgttcagcagtttggcaatcaactggcgaaactggaaccgattgtttcggtattgcagagcgacccggaacagttcgaacagttaaaagaagattacgcgtactctcagcagatgcagcgcgatgcccgtcagcaggcgtttgccctgacggaagtggtgcagcgtcgtgcgcactttagctattctgactcggcagaaatgcttagcggtaacagcgatctcaacgaaaaactgcgtgaacgtctggaacaggcggaagcggagcgtacccgcgctcgcgaagcgttgcgcggtcacgcagcgcagttgagtcagtacaaccaggtgctggcttcgctgaaaagttcttacgacaccaaaaaagagctactcaacgatctgcaacgtgaattgcaggatatcggcgtgcgtgctgatagcggggcagaagagcgggcgcgtattcgccgtgacgagctgcatgcgcaactgagcaataaccgttcacgccgcaatcaactggaaaaagcgcttaccttctgcgaagcggagatggacaacctgacccgcaaactgcgcaagctggagcgggattactttgagatgcgcgagcaggtagtgaccgccaaagcgggctggtgtgcggtgatgcgcatggtgaaagataacggcgttgagcgccgcttacaccgtcgtgagctggcttatctctccgctgatgatttgcgttccatgtcggataaggcgttaggtgcgctgcgtctggcggtggcggataacgaacatctgcgcgacgtgctgcgcatgtcggaagatccgaaacgtccggagcgtaaaattcagttcttcgtggcggtttatcagcatctgcgtgaacgtattcgtcaggatattattcgtaccgatgatccggtggaagctatcgaacagatggagattgaacttagccgtctgaccgaagaattaacctcccgtgaacagaaactggcgatcagttcccgcagcgtggcgaacatcattcgcaaaaccattcagcgcgagcagaaccgtatccgtatgctcaaccaggggttgcagaacgtatcgtttggtcaggtgaacagcgtgcgtctcaacgtgaacgtgcgtgaaacgcacgccatgctactggatgtgctctctgaacagcacgagcagcatcaggatctgtttaacagcaaccgtttgaccttctcggaagcgctggcgaaactgtatcaacgtcttaacccgcagattgatatggggcagcgcacgccgcagaccatcggtgaagaactgctggattaccgcaactatctggaaatggaagttgaggttaaccgtggttccgatggctggctgcgcgcagagtctggtgcattgtcgaccggtgaggcgattggtaccggtatgtcgattctggtgatggtggtacaaagctgggaagatgaatctcgccgcctgcgcggtaaagatatctctccttgccgcctgctgttcctcgatgaagcagcgcgactggatgctcgttctatcgccacgctgtttgaattgtgtgagcgtttgcaaatgcaactcatcatcgcagcgccggaaaatatcagcccggagaaaggcaccacctataaactggtgcgtaaagtcttccagaataccgaacacgttcatgtcgtcggcctgcgaggatttgcgccgcaactccctgaaacgcttccaggaactgacgaagcgccttctcaggcgagttaa

SEQ ID NO: 45 - DNA, 3186 bp, synthetic construct
  source 1..3186: mol_type = other DNA
atgaaaagaatgttaatcaacgcaactcagcaggaagagttgcgcgttgcccttgtagatgggcagcgtctgtatgacctggatatcgaaagtccagggcacgagcagaaaaaggcaaacatctacaaaggtaaaatcacccgcattgaaccgagtctggaagctgcttttgttgattacggcgctgaacgtcacggtttcctcccactaaaagaaattgcccgcgaatatttccctgctaactacagtgctcatggtcgtcccaacattaaagatgtgttgcgtgaaggtcaggaagtcattgttcagatcgataaagaagagcgcggcaacaaaggcgcggcattaaccacctttatcagtctggcgggtagctatctggttctgatgccgaacaacccgcgcgcgggtggcatttctcgccgtatcgaaggcgacgaccgtaccgaattaaaagaagcactggcaagccttgaactgccggaaggcatggggcttatcgtgcgcaccgctggcgtcggcaaatctgctgaggcgctgcaatgggatttaagcttccgtctgaaacactgggaagccatcaaaaaagccgctgaaagccgcccggccccgttcctgattcatcaggagagcaacgtaatcgttcgcgcattccgcgattacttacgtcaggacatcggcgaaatccttatcgataacccgaaagtgctcgaactggcacgtcagcatatcgctgcattaggtcgcccggatttcagcagcaaaatcaaactgtacaccggcgagatcccgctgttcagccactaccagatcgagtcacagatcgagtccgccttccagcgtgaagttcgtctgccgtctggtggttccattgttatcgacagcaccgaagcgttaacggccatcgacatcaactccgcacgcgcgacccgcggcggcgatatcgaagaaaccgcgtttaacactaacctcgaagctgccgatgagattgctcgtcagctgcgcctgcgtgacctcggcggcctgattgttatcgacttcatcgacatgacgccagtacgccaccagcgtgcggtagaaaaccgtctgcgtgaagcggtgcgtcaggaccgtgcgcgtattcaaatcagccatatttctcgctttggcctgctggaaatgtcccgtcagcgcctgagcccatcactgggtgaatccagccatcacgtctgcccgcgctgctccggtaccggtaccgtgcgtgacaacgaatcgctgtcgctctctattctgcgtctgatcgaagaagaagcgctgaaagagaacacccaggaagttcacgccattgttcctgtgccaatcgcttcttatctgctgaatgaaaaacgttctgcggtgaatgccattgaaacgcgtcaggacggtgttcgctgcgtgattgtgccaaacgatcagatggaaaccccgcactaccacgtgctgcgcgtgcgtaaaggggaagaaacgccgaccttaagctacatgctgccgaagctgcatgaagaagcgatggcgctgccgtctgaagaagagttcgctgaacgtaagcgtccggaacaacctgcgctggcaacctttgccatgccggatgtgccgccagcgccaaccccagctgaacctgccgcgcctgtcgtagccccagcacctaaatctgcaccggcaacaccagccgctcctgcccaacctgggctgttgagccgcttcttcggcgcactgaaagcgctgttcagcggtggtgaagaaaccaaaccgtccgagcaaccaacaccgaaagcagaagcgaaaccggaacgtcaacaggatcgtcgcaagcctcgtcagaacaaccgccgtgaccgtaatgagcgccgcgacacccgtagtgaacgtactgaaggcagcgataatcgcgaagaaaaccgtcgtaatcgtcgccaggcacagcagcagactgccgagacgcgtgagagccgtcagcaggttgaggtaacggaaaaagcgcgtaccaccgacgagcagcaagcgccgcgtcgtgaacgtagccgccgccgtaatgatgataaacgtcaggcgcaacaagaagcgaaggcgctgaatgttgaagagcaatctgttcaggaaaccgaacaggaagaacgtgtacgtccggttcagccgcgtcgtaaacagcgtcagctcaatcagaaagtgcgttacgagcaaagcgtagccgaagaagcggtagtcgcaccggtggttgaagaaactgtcgctgccgaaccaattgttcaggaagcgccagctccacgcacagaactggtgaaagtcccgctgccagtcgtagcgcaaactgcaccagaacagcaagaagagaacaatgctgataaccgtgacaacggtggcatgccgcgtcgttctcgccgctcgcctcgtcacctgcgcgtaagtggtcagcgtcgtcgtcgctatcgtgacgagcgttatccaacccagtcgccaatgccgttgaccgtagcgtgcgcgtctccggaactggcctctggcaaagtctggatccgctatccaattgtacgtccgcaagatgtacaggttgaagagcagcgcgaacaggaagaagtacatgtgcagccgatggtgactgaggtccctgtcgccgccgctatcgaaccggttgttagcgcgccagttgttgaagaagtggccggtgtcgtagaagcccccgttcaggttgccgaaccgcaaccggaagtggttgaaacgacgcatcctgaagtgattgctgccgcggtaactgaacagccgcaggtgattaccgagtctgatgttgccgtagcccaggaagttgcagaacaagcagaaccggtggttgaaccgcaggaagagacggcagatattgaagaagttgtcgaaactgctgaggttgtagttgctgaacctgaagttgttgctcaacctgccgcgccagtagtcgctgaagtcgcagcagaagttgaaacggtagctgcggtcgaacctgaggtcaccgttgagcataaccacgctaccgcgccaatgacgcgcgctccagcaccggaatatgttccggaggcaccgcgtcacagtgactggcagcgccctacttttgccttcgaaggtaaaggtgccgcaggtggtcatacggcaacacatcatgcctctgctgctcctgcgcgtccgcaacctgttgagtaa

SEQ ID NO: 46 - RNA, 24 nt, synthetic construct
  source 1..24: mol_type = other RNA
agatcgaaagcgtatccggtgaaa

SEQ ID NO: 47 - RNA, 24 nt, synthetic construct
  source 1..24: mol_type = other RNA
atactttcaccggatacgctttcg

SEQ ID NO: 48 - RNA, 24 nt, synthetic construct
  source 1..24: mol_type = other RNA
agattatatgagcgataaaattat

SEQ ID NO: 49 - RNA, 24 nt, synthetic construct
  source 1..24: mol_type = other RNA
atacataattttatcgctcatata

SEQ ID NO: 50 - RNA, 24 nt, synthetic construct
  source 1..24: mol_type = other RNA
agattggttcgcctggtatccttt

SEQ ID NO: 51 - RNA, 24 nt, synthetic construct
  source 1..24: mol_type = other RNA
atacaaaggataccaggcgaacca

SEQ ID NO: 52 - RNA, 24 nt, synthetic construct
  source 1..24: mol_type = other RNA
agataaggccgcgtagcaacgtcg

SEQ ID NO: 53 - RNA, 24 nt, synthetic construct
  source 1..24: mol_type = other RNA
ataccgacgttgctacgcggcctt

SEQ ID NO: 54 - RNA, 24 nt, synthetic construct
  source 1..24: mol_type = other RNA
agatctgcaaaaagcagcacaagg

SEQ ID NO: 55 - RNA, 24 nt, synthetic construct
  source 1..24: mol_type = other RNA
atacccttgtgctgctttttgcag

SEQ ID NO: 56 - RNA, 24 nt, synthetic construct
  source 1..24: mol_type = other RNA
agatagatgaccgatctggatctt

SEQ ID NO: 57 - RNA, 24 nt, synthetic construct
  source 1..24: mol_type = other RNA
atacaagatccagatcggtcatct

SEQ ID NO: 58 - RNA, 24 nt, synthetic construct
  source 1..24: mol_type = other RNA
agattctttcttcgtctaaagatt

SEQ ID NO: 59 - RNA, 24 nt, synthetic construct
  source 1..24: mol_type = other RNA
atacaatctttagacgaagaaaga

SEQ ID NO: 60 - RNA, 24 nt, synthetic construct
  source 1..24: mol_type = other RNA
agatgtgaccaacgtagataaagc

SEQ ID NO: 61 - RNA, 24 nt, synthetic construct
  source 1..24: mol_type = other RNA
atacgctttatctacgttggtcac

SEQ ID NO: 62 - RNA, 24 nt, synthetic construct
  source 1..24: mol_type = other RNA
agattgccagactgtaagtacccg

SEQ ID NO: 63 - RNA, 24 nt, synthetic construct
  source 1..24: mol_type = other RNA
ataccgggtacttacagtctggca

SEQ ID NO: 64 - RNA, 24 nt, synthetic construct
  source 1..24: mol_type = other RNA
agataagccgaagtacatctggag

SEQ ID NO: 65 - RNA, 24 nt, synthetic construct
  source 1..24: mol_type = other RNA
atacctccagatgtacttcggctt

SEQ ID NO: 66 - RNA, 24 nt, synthetic construct
  source 1..24: mol_type = other RNA
agattcggaactggatatgatggt

SEQ ID NO: 67 - RNA, 24 nt, synthetic construct
  source 1..24: mol_type = other RNA
atacaccatcatatccagttccga

SEQ ID NO: 68 - RNA, 24 nt, synthetic construct
  source 1..24: mol_type = other RNA
agatgctcactgacgctgattaac

SEQ ID NO: 69 - RNA, 24 nt, synthetic construct
  source 1..24: mol_type = other RNA
atacgttaatcagcgtcagtgagc

SEQ ID NO: 70 - RNA, 24 nt, synthetic construct
  source 1..24: mol_type = other RNA
agataaatgcacaaaacttttata

SEQ ID NO: 71 - RNA, 24 nt, synthetic construct
  source 1..24: mol_type = other RNA
atactataaaagttttgtgcattt

SEQ ID NO: 72 - RNA, 24 nt, synthetic construct
  source 1..24: mol_type = other RNA
agatcagcgcagcgcgcgagctga

SEQ ID NO: 73 - RNA, 24 nt, synthetic construct
  source 1..24: mol_type = other RNA
atactcagctcgcgcgctgcgctg

SEQ ID NO: 74 - RNA, 24 nt, synthetic construct
  source 1..24: mol_type = other RNA
agatttaaggttatcatccgtttc

SEQ ID NO: 75 - RNA, 24 nt, synthetic construct
  source 1..24: mol_type = other RNA
atacgaaacggatgataaccttaa

SEQ ID NO: 76 - RNA, 24 nt, synthetic construct
  source 1..24: mol_type = other RNA
agatgcctgtgtaatctcccttac

SEQ ID NO: 77 - RNA, 24 nt, synthetic construct
  source 1..24: mol_type = other RNA
atacgtaagggagattacacaggc

SEQ ID NO: 78 - RNA, 24 nt, synthetic construct
  source 1..24: mol_type = other RNA
agatctgtatatactcacagcata

SEQ ID NO: 79 - RNA, 24 nt, synthetic construct
  source 1..24: mol_type = other RNA
atactatgctgtgagtatatacag

SEQ ID NO: 80 - RNA, 24 nt, synthetic construct
  source 1..24: mol_type = other RNA
agatatctcatccgtgatcacatc

SEQ ID NO: 81 - RNA, 24 nt, synthetic construct
  source 1..24: mol_type = other RNA
atacgatgtgatcacggatgagat

SEQ ID NO: 82 - RNA, 24 nt, synthetic construct
  source 1..24: mol_type = other RNA
agatgctttaaaatccgcgagcac

SEQ ID NO: 83 - RNA, 24 nt, synthetic construct
  source 1..24: mol_type = other RNA
atacgtgctcgcggattttaaagc

SEQ ID NO: 84 - RNA, 24 nt, synthetic construct
  source 1..24: mol_type = other RNA
agattcgtataatggattacggca

SEQ ID NO: 85 - RNA, 24 nt, synthetic construct
  source 1..24: mol_type = other RNA
atactgccgtaatccattatacga

SEQ ID NO: 86 - RNA, 24 nt, synthetic construct
  source 1..24: mol_type = other RNA
agatcgctagtttcacccgggggc

SEQ ID NO: 87 - RNA, 24 nt, synthetic construct
  source 1..24: mol_type = other RNA
atacgcccccgggtgaaactagcg

SEQ ID NO: 88 - RNA, 24 nt, synthetic construct
  source 1..24: mol_type = other RNA
agatttgtcgaacgcgaacggctg

SEQ ID NO: 89 - RNA, 24 nt, synthetic construct
  source 1..24: mol_type = other RNA
ataccagccgttcgcgttcgacaa

SEQ ID NO: 90 - RNA, 24 nt, synthetic construct
  source 1..24: mol_type = other RNA
agataaggcatagtttaccatgcg

SEQ ID NO: 91 - RNA, 24 nt, synthetic construct
  source 1..24: mol_type = other RNA
ataccgcatggtaaactatgcctt

SEQ ID NO: 92 - RNA, 24 nt, synthetic construct
  source 1..24: mol_type = other RNA
agatcactggagcgcaatgccgcc

SEQ ID NO: 93 - RNA, 24 nt, synthetic construct
  source 1..24: mol_type = other RNA
atacggcggcattgcgctccagtg

SEQ ID NO: 94 - RNA, 24 nt, synthetic construct
  source 1..24: mol_type = other RNA
agatgactggtatcaggcggatga

SEQ ID NO: 95 - RNA, 24 nt, synthetic construct
  source 1..24: mol_type = other RNA
atactcatccgcctgataccagtc

SEQ ID NO: 96 - RNA, 24 nt, synthetic construct
  source 1..24: mol_type = other RNA
agattcgtgaattacgtgagcaag

SEQ ID NO: 97 - RNA, 24 nt, synthetic construct
  source 1..24: mol_type = other RNA
ataccttgctcacgtaattcacga

SEQ ID NO: 98 - RNA, 24 nt, synthetic construct
  source 1..24: mol_type = other RNA
agattgatttaatagatagtattc

SEQ ID NO: 99 - RNA, 24 nt, synthetic construct
  source 1..24: mol_type = other RNA
atacgaatactatctattaaatca

SEQ ID NO: 100 - RNA, 24 nt, synthetic construct
  source 1..24: mol_type = other RNA
agatatcgctatgtcctggccctc

SEQ ID NO: 101 - RNA, 24 nt, synthetic construct
  source 1..24: mol_type = other RNA
atacgagggccaggacatagcgat

SEQ ID NO: 102 - RNA, 24 nt, synthetic construct
  source 1..24: mol_type = other RNA
agatggctaccgtagagtcctcgg

SEQ ID NO: 103 - RNA, 27 nt, synthetic construct
  source 1..27: mol_type = other RNA
atacttgggctaccgtagagtcctcgg

SEQ ID NO: 104 - RNA, 24 nt, synthetic construct
  source 1..24: mol_type = other RNA
agatgctctggcctagaggacaag

SEQ ID NO: 105 - RNA, 24 nt, synthetic construct
  source 1..24: mol_type = other RNA
ataccttgtcctctaggccagagc

SEQ ID NO: 106 - RNA, 24 nt, synthetic construct
  source 1..24: mol_type = other RNA
agattccctatcaagcattctgac

SEQ ID NO: 107 - RNA, 24 nt, synthetic construct
  source 1..24: mol_type = other RNA
atacgtcagaatgcttgataggga

SEQ ID NO: 108 - RNA, 24 nt, synthetic construct
  source 1..24: mol_type = other RNA
agatacccttgaagtcactgcccg

SEQ ID NO: 109 - RNA, 24 nt, synthetic construct
  source 1..24: mol_type = other RNA
ataccgggcagtgacttcaagggt

SEQ ID NO: 110 - RNA, 24 nt, synthetic construct
  source 1..24: mol_type = other RNA
agatcagtggaataatgaggccgt

SEQ ID NO: 111 - RNA, 24 nt, synthetic construct
  source 1..24: mol_type = other RNA
atacacggcctcattattccactg

SEQ ID NO: 112 - RNA, 24 nt, synthetic construct
  source 1..24: mol_type = other RNA
agatttgattacggcgctgaacgt

SEQ ID NO: 113 - RNA, 24 nt, synthetic construct
  source 1..24: mol_type = other RNA
atacacgttcagcgccgtaatcaa

SEQ ID NO: 114 - RNA, 24 nt, synthetic construct
  source 1..24: mol_type = other RNA
agatcgtctgtaaattccctacaa

SEQ ID NO: 115 - RNA, 24 nt, synthetic construct
  source 1..24: mol_type = other RNA
atacttgtagggaatttacagacg

SEQ ID NO: 116 - RNA, 24 nt, synthetic construct
  source 1..24: mol_type = other RNA
agataaactgagatcatttttgat

SEQ ID NO: 117 - RNA, 24 nt, synthetic construct
  source 1..24: mol_type = other RNA
atacatcaaaaatgatctcagttt

SEQ ID NO: 118 - RNA, 24 nt, synthetic construct
  source 1..24: mol_type = other RNA
agatgcacactgggcaatacgagg

SEQ ID NO: 119 - RNA, 24 nt, synthetic construct
  source 1..24: mol_type = other RNA
ataccctcgtattgcccagtgtgc

SEQ ID NO: 120 - protein, 6 aa, synthetic construct
HHHHHH

(19) United States
(12) Patent Application Publication
(10) Pub. No.: US 2025/0283053 A1
(43) Pub. Date: Sep. 11, 2025

(54) ENHANCED CELL-FREE BACTERIOPHAGE SYNTHESIS BY GENETIC MODULATION OF BACTERIAL TRANSCRIPTION/TRANSLATION MACHINERY (TXTL) MACHINERY

(71) Applicant: The Administrators of the Tulane Educational Fund, New Orleans, LA (US)

(72) Inventor: Rani Brooks, New Orleans, LA (US)

(73) Assignee: The Administrators of the Tulane Educational Fund, New Orleans, LA (US)

(21) Appl. No.: 18/858,248

(22) PCT Filed: Apr. 19, 2023
(86) PCT No.: PCT/US2023/019145

§ 371 Date: Oct. 18, 2024

Related U.S. Application Data
(60) Provisional application No. 63/332,901, filed on Apr. 20, 2022.

(51) Int. Cl. C12N 7/00; C12N 1/06; C12N 1/20; C12N 9/22; C12N 15/11; C12N 15/70; C12R 1/19

(52) U.S. Cl. CPC C12N 7/00; C12N 1/066; C12N 1/20; C12N 9/226; C12N 15/111; C12N 15/70; C12N 2310/20; C12N 2795/00051; C12R 2001/19

(57) ABSTRACT

The present disclosure relates to compositions including or obtained from genetically modified bacterial host cells (e.g., E. coli) and methods for using the same for cell-free bacteriophage synthesis (CFBS). In particular, the present technology relates to genetically modified E. coli that overexpress one or more of translation initiation factor IF-3 (infC), OxyS and CyaR and/or repress RecC subunit exonuclease RecBCD, and methods for using the same to obtain improved CFBS yields.

### CROSS-REFERENCE TO RELATED APPLICATIONS

This application claims the benefit of and priority to U.S. Provisional Patent Application No. 63/332,901, filed Apr. 20, 2022, the contents of which are incorporated herein by reference in its entirety.

### GOVERNMENT SUPPORT

This invention was made with government support under W81XWH-20-1-0071 awarded by the Other Agency, and 1144646 awarded by the National Science Foundation. The government has certain rights in the invention.

### TECHNICAL FIELD

The present disclosure relates to compositions including or obtained from genetically modified bacterial host cells (e.g., E. coli) and methods for using the same for cell-free bacteriophage synthesis (CFBS). In particular, the present technology relates to genetically modified E. coli that overexpress one or more of translation initiation factor IF-3 (infC), OxyS and CyaR and/or repress RecC subunit of the RecBCD exonuclease, and methods for using the same to obtain improved CFBS yields.

### BACKGROUND

The following description of the background of the present technology is provided simply as an aid in understanding the present technology and is not admitted to describe or constitute prior art to the present technology.

Multidrug-resistant (MDR) bacteria pose one of the greatest emerging health threats. These pathogens possess intrinsic and acquired mechanisms of resistance to most common antibiotics, leaving few treatment options for the most at-risk patients. The Centers for Disease Control reports 2.8 million MDR infections per year leading to 35,000 deaths with numbers are on the rise². Phage therapy, a resurgent anti-bacterial treatment, has the potential to cure bacterial infections resistant to small molecule antibiotics. Unlike small molecule antibiotics, phage do not harm beneficial bacterial flora that protect against future infections.

Despite a promising future, phage therapy is encumbered by logistical challenges in the manufacturing process. Once a suitable phage has been identified for a particular application, it needs to be propagated using a working host, which ideally is well characterized, non-pathogenic, and free of genomically encoded prophage to prevent potential contamination of phage preparations with lysogenic phage⁶. Such a host is often not immediately available. Next, crude phage preparations must be purified to remove endotoxin, a component of outer membranes of Gram-negative bacteria that induces septic shock⁷. While several organic solvent extraction or affinity column purification methods exist to remove endotoxin, these approaches have widely varying efficiencies depending on the phage undergoing purification and typically result in significant loss of titer⁸. Additionally, low shelf-stability can result in a significant loss of titer prior to clinical administration due to storage conditions, shipping conditions, and phage-dependent variability⁹⁻¹¹.

Accordingly, there is an urgent need for phage production methods that achieve enhanced yields over conventional cell-free bacteriophage synthesis (CFBS) methods.

### SUMMARY OF THE PRESENT TECHNOLOGY

In one aspect, the present disclosure provides genetically modified bacterial host cells (e.g., E. coli) that overexpress one or more of translation initiation factor IF-3 (infC), OxyS and CyaR and/or comprise a vector that includes a nucleic acid sequence encoding a CRISPR enzyme and a vector that includes a nucleic acid sequence encoding a crRNA that specifically targets a promoter region located upstream of a transcription start site (TSS) of recC or a recC coding region located downstream of a transcription start site (TSS) of recC.

In some embodiments, the genetically modified bacterial host cells comprise a non-endogenous expression vector that comprises a translation initiation factor IF-3 (infC) nucleic acid sequence of SEQ ID NO: 39, an OxyS nucleic acid sequence of SEQ ID NO: 41, a CyaR nucleic acid sequence of SEQ ID NO: 42, or any combination thereof.

Additionally or alternatively, in some embodiments, the non-endogenous expression vector comprising the infC nucleic acid sequence and the non-endogenous expression vector comprising the OxyS nucleic acid sequence and/or the CyaR nucleic acid sequence are the same or distinct. In other embodiments, the non-endogenous expression vector comprising the OxyS nucleic acid sequence and the non-endogenous expression vector comprising the CyaR nucleic acid sequence are the same or distinct.

In any of the preceding embodiments of the genetically modified bacterial host cells disclosed herein, the non-endogenous expression vector comprising the infC nucleic acid sequence, and/or the non-endogenous expression vector comprising the OxyS nucleic acid sequence, and/or the non-endogenous expression vector comprising the CyaR nucleic acid sequence is a plasmid, a cosmid, a bacmid, a bacterial artificial chromosome (BAC), or a viral vector. In certain embodiments, the non-endogenous expression vector comprising the infC nucleic acid sequence, and/or the non-endogenous expression vector comprising the OxyS nucleic acid sequence, and/or the non-endogenous expression vector comprising the CyaR nucleic acid sequence is operably linked to an expression control sequence. The expression control sequence may be an inducible promoter, a constitutive promoter, an endogenous promoter, or a heterologous promoter.

Additionally or alternatively, in some embodiments, the genetically modified bacterial host cells of the present technology comprise a vector that includes a nucleic acid sequence encoding a CRISPR enzyme and a vector that includes a nucleic acid sequence encoding a crRNA that specifically targets a promoter region located upstream of a transcription start site (TSS) of recC or a recC coding region located downstream of a transcription start site (TSS) of recC. In some embodiments, the vector including the nucleic acid sequence encoding the CRISPR enzyme and the vector including the nucleic acid sequence encoding the cRNA are the same or distinct. In certain embodiments, the nucleic acid sequence encoding the CRISPR enzyme is operably linked to an inducible promoter and/or the nucleic acid sequence encoding the crRNA is operably linked to a constitutive promoter.

Additionally or alternatively, in certain embodiments of the genetically modified bacterial host cells of the present technology, the CRISPR enzyme is a nuclease-deficient Francisella novicida Cas12a (dFnCas12a) and/or the nucleic acid sequence encoding the crRNA is selected from the group consisting of SEQ ID NO: 86, SEQ ID NO: 87, SEQ ID NO: 88 and SEQ ID NO: 89.

Additionally or alternatively, in some embodiments, the genetically modified bacterial host cells of the present technology comprise a deletion or mutation in (a) a promoter region located upstream of a transcription start site (TSS) of recC. or (b) a recC coding region located downstream of a transcription start site (TSS) of recC, wherein the mutation or deletion represses transcription of recC. The deletion may be located upstream or downstream of the transcription start site (TSS) of recC. In some embodiments, the deletion is about 10-about 100 base pairs (bps) in length. In other embodiments, the mutation is a nonsense mutation, a frameshift mutation, or a missense mutation.

In any of the foregoing embodiments described herein, the genetically modified bacterial host cells are E. coli cells.

In one aspect, the present disclosure provides a cell lysate obtained from any and all embodiments of the genetically modified bacterial host cells disclosed herein. The cell lysates of the present technology comprise an effective amount of transcription/translation (TXTL) machinery that is configured to synthesize bacteriophage under cell-free conditions. The cell lysates may be prepared using one or more of French-press cell lysis, sonication, runoff reactions, or lysate dialysis.

In another aspect, the present disclosure provides an in vitro method for synthesizing bacteriophage virions comprising contacting a bacteriophage genome with any and all embodiments of the cell lysates disclosed herein and an energy buffer in vitro to obtain a reaction mixture, and incubating the reaction mixture under conditions to produce viable phage virions, wherein the energy buffer comprises canonical amino acids, phosphoenol pyruvate (PEP), nucleoside triphosphates (NTPs), cofactors, and coenzymes. In some embodiments, the reaction mixture is incubated at 16° C. to 42° C. for about 1-24 hours. The bacteriophage genome may be derived from a naturally occurring bacteriophage, or a genetically engineered bacteriophage. In some embodiments, the bacteriophage genome is obtained from a bacteriophage that specifically infects the naturally occurring counterpart of the genetically modified bacterial host cells disclosed herein, such as E coli. Additionally or alternatively, in some embodiments, the bacteriophage genome is obtained from a bacteriophage selected from among T7 phage, T7-like phage, Lambda, K1E, T3, T5, T4, or PhiX174.

Additionally or alternatively, in some embodiments, the energy buffer further comprises one or more of a polyethylene glycol (PEG) polymer (such as PEG-8000 or PEG-6000), deoxynucleotide triphosphates (dNTPs), and stabilizers. In certain embodiments, the energy buffer further comprises one or more of Mg-glutamate, K-glutamate, tRNA, cAMP, folinic acid, spermidine, 3-PGA, HEPES, Nicotinamide adenine dinucleotide (NAD), coenzyme A (CoA), DTT, and maltodextrin.

In any of the preceding embodiments of the methods disclosed herein, the reaction mixture further comprises a vector including a nucleic acid sequence encoding a reporter gene, wherein the nucleic acid sequence encoding the reporter gene is operably linked to a promoter that is responsive to a RNA polymerase (RNAP) encoded by the bacteriophage genome. In certain embodiments, the RNAP is selected from among SP6 RNA Polymerase, T3 RNA Polymerase and T7 RNA polymerase. The reporter gene may encode a bioluminescent protein, a fluorescent protein, or a chemiluminescent protein. Examples of bioluminescent proteins include, but are not limited to, Aequorin, firefly luciferase, Renilla luciferase, red luciferase, luxAB, or nanoluciferase. Examples of chemiluminescent proteins include, but are not limited to, β-galactosidase, horseradish peroxidase (HRP), or alkaline phosphatase. Examples of fluorescent proteins include, but are not limited to, sfGFP, TagBFP, Azurite, EBFP2, mKalamal, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOx, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherryl, PATagRFP, Kaede (green), Kaede (red), KikGR1 (green), KikGR1 (red), PS-CFP2, PS-CFP2, mEos2 (green), mEos2 (red), PSmOrange, or Dronpa.

Additionally or alternatively, in some embodiments of the methods disclosed herein, the phage virions show increased efficiency of plating (EOP) relative to phage virions obtained with cell-free bacteriophage synthesis (CFBS) in a control lysate obtained from a wild-type bacterial cell, wherein the wild-type bacterial cell and the genetically modified bacterial host cell are the same species.

In one aspect, the present disclosure provides a kit comprising one or more expression vectors comprising at least one nucleic acid sequence selected from the group consisting of SEQ ID NO: 39, SEQ ID NO: 41 and SEQ ID NO: 42, and instructions for using the same to prepare donor bacterial cell lysates for cell-free bacteriophage synthesis (CFBS). In certain embodiments, the at least one nucleic acid sequence is operably linked to an inducible promoter, a constitutive promoter, an endogenous promoter, or a heterologous promoter. Additionally or alternatively, in some embodiments, the kits further comprise vials containing naturally-occurring or non-natural bacterial host cells that can be transformed with the one or more expression vectors comprising at least one nucleic acid sequence selected from the group consisting of SEQ ID NO: 39, SEQ ID NO: 41 and SEQ ID NO: 42. In some embodiments, the bacterial host cells are E. coli.

In another aspect, the present disclosure provides a kit comprising one or more expression vectors comprising at least one nucleic acid sequence encoding a crRNA that specifically targets a promoter region located upstream of a transcription start site (TSS) of recC or a recC coding region located downstream of a transcription start site (TSS) of recC, and instructions for using the same to prepare donor bacterial cell lysates for cell-free bacteriophage synthesis (CFBS). In some embodiments, the one or more expression vectors further comprise a nucleic acid sequence encoding a CRISPR enzyme. Examples of CRISPR enzymes include, but are not limited to, nuclease-deficient Cas12a from F. novicida (dFnCas12a), Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4. Additionally or alternatively, in some embodiments, the nucleic acid sequence encoding the CRISPR enzyme is operably linked to an inducible promoter and/or the at least one nucleic acid sequence encoding the crRNA is operably linked to a constitutive promoter. In some embodiments, the nucleic acid sequence encoding the crRNA is selected from the group consisting of SEQ ID NO: 86, SEQ ID NO: 87, SEQ ID NO: 88 and SEQ ID NO: 89. Additionally or alternatively, in some embodiments, the kits further comprise vials containing naturally-occurring or non-natural bacterial host cells that can be transformed with the one or more expression vectors comprising the nucleic acid sequence encoding the CRISPR enzyme and/or the at least one nucleic acid sequence encoding the crRNA. In some embodiments, the bacterial host cells are E. coli.

In yet another aspect, the present disclosure provides a kit comprising any and all embodiments of the genetically modified bacterial host cell described herein, and instructions for using the same to prepare donor bacterial cell lysates for cell-free bacteriophage synthesis (CFBS). In some embodiments, the genetically modified bacterial host cells are E. coli.

In any and all embodiments of the kits disclosed herein, the kits further comprise one or more of polyethylene glycol (PEG) polymers (e.g., PEG-8000, PEG-6000), deoxynucleotide triphosphates (dNTPs), stabilizers, and an energy buffer comprising canonical amino acids, phosphoenol pyruvate (PEP), nucleoside triphosphates (NTPs), cofactors, and coenzymes.

### BRIEF DESCRIPTION OF THE DRAWINGS

FIGS. 1A-1B: Gene expression background in TXTL source influences CFBS yields. FIG. 1A: The number of progeny resulting from bacteriophage T7 infection of host E. coli BL21 is influenced by the genetic background of the host at the time of infection. Knockdown (KD) or overexpression (OX) of certain genes may positive, negative, or neutral impact on T7 progeny yield relative to wild-type BL21 (WT). FIG. 1B: Cell-free bacteriophage synthesis (CFBS) can be modulated by modifying the genetic background of the source of transcription/translation (TXTL) machinery derived from cell lysates.

FIGS. 2A-2C: Inducible CRISPRi of trxA knockdown mRNA and T7 efficiency of plating (EOP). Induced expression of (FIG. 2A) dFnCas12a with crRNA targeting the trxA promotor results in repression of (FIG. 2B) trxA by 90±2.4%, which is associated with a (FIG. 2C) significant efficiency of plating (EOP) reduction to 12±7.0%. Data represented as mean SD (n=3). Rhamnose induction significantly lowers trxA expression vs. the NT-control (p<0.0001, 2-Way ANOVA). Welch's two-tailed t-test was performed indicating significant reduction of EOP (p<0.05).

FIGS. 3A-3C: CRISPRi-mediated gene repression in E. coli BL21 has varied impact on T7 lysis onset time and mean lysis time (T50%). CRISPRi induced by 2% (w/v) L-rhamnose for 4 h changes lysis profiles of T7 infecting log-phase E. coli BL21 depending on gene target and whether crRNA targets promotors (odd numbers) or coding sequences (even numbers). A non-targeting control was included in each experiment (g037). Optical densities (OD) were normalized with max OD=1 for clearer comparisons. Shaded regions represent max and min OD of triplicate experiments performed on different days. Representative curve effects (FIG. 3A): neutral (NT-control), shift (trxA (g001)), widening (mukB (g012)), and shoulder (infC (g020)). Lysis onset time (FIG. 3B) and mean lysis time (FIG. 3C) are represented as mean±SD (n=3). Welch's two-tailed t-test was performed indicating significant change in lysis and mean lysis timing for as a result of CRISPRi induction (p<0.05).

FIGS. 4A-4B: Gene overexpression and CRISPRi-mediated gene repression modulates efficiency of plating (EOP). Log-phase E. coli BL21 knockdown (FIG. 4A) and overexpression (FIG. 4B) strains were infected with phage T7 at MOI=0.0001. After 30 min (roughly once lysis cycle) at 37° C., further phage replication was stopped by adding chloroform to each culture followed by pipette mixing. Phage yields were enumerated by plaque counting on double-layer agar plates containing E. coli BL21 as propagation host. Efficiency of plating (EOP) represents the fraction change in assembled phage between (FIG. 4A) gene-repressed CRISPRi strains and their non-repressed counterparts carrying the same plasmid without CRISPRi induction or (FIG. 4B) fraction change in assembled phage between overexpressing strains and their non-induced counterparts carrying the same plasmid. Data represented as mean±SD (n=3). Welch's two-tailed t-test was performed indicating significant change in EOP as a result of CRISPRi induction (p<0.05). p<0.5, *; p<0.01, **; p<0.001, ***; p<0.0001, ****.

FIGS. 5A-5B: T7 genome pJl1-T7-Pr-sfGFP cascade phage-dependent TXTL mechanism. A cell-free expression system synthesizing sfGFP was used to troubleshoot CFBS reaction. (FIG. 5A) 0.5 nM T7 genome was included in a standard CFES reaction using sfGFP expression controlled by T7 RNA polymerase (RNAP). (FIG. 5B) Transcription of sfGFP only occurs if T7 RNAP (T7 gene 1) itself is transcribed by endogenous E. coli RNAP. sfGFP expression reflects functional endogenous TXTL capacity and T7 RNAP synthesis and transcription activity.

FIGS. 6A-6C: Cell-free protein and bacteriophage synthesis yields are influenced by genetic background of TXTL donor. Biological duplicates (1 and 2) of cell lysates were prepared from E. coli BL21 overexpression (UP) or repression (DOWN) of various effector genes. Protein synthesis (FIG. 6A) was carried out using the T7 genome pJl1-T7-Pr-sfGFP cascade. (FIG. 6B) CFBS yields were measure by plaque count assays using wild-type BL21 as propagation host. Gray bars indicate protein and phage yields at 4 h. Black dotted lines indicate 4 h yields in wild-type BL21 lysates and colored lines indicate 20 h yields. (FIG. 6C) Relative sfGFP fluorescence and T7 titers shown as heatmaps. Data shows mean±SD (n=3). Welch's two-tailed t-test was performed indicating significant increase in sfGFP fluoresce or T7 titer relative to the wild-type E. coli BL21 baseline (p<0.05).

FIGS. 7A-7B: Comparison of cell-free systems for protein or phage synthesis. In vitro synthesis of (FIG. 7A) fluorescent reporter protein sfGFP and cell-free bacteriophage synthesis (FIG. 7B) of phage T7. Each system contains transcription/translation (TXTL) machinery derived from E. coli BL21 lysates, a DNA template, and energy buffer (20 canonical amino acids, phosphoenol pyruvate (PEP), NTPs, cofactors, and coenzymes). sfGFP in encoded on a circular plasmid (a) and phage T7 expression encoded on purified T7 gDNA. CFBS reactions are supplemented with molecular crowder PEG-8000 and dNTPs to support genome replication.

FIG. 8: CRISPRi/overexpression induction experimental workflow. Phenotypic readouts for gene repression and overexpression on T7 fitness included impacts on lysis curve profile (e.g. lysis timing, mean lysis timing) and efficiency of plating. Gene repression was validated by RT-qPCR.

FIG. 9: Single-plasmid CRISPR interference (CRISPRi) vector pCRJ001.

FIGS. 10A-10B: CRISPRi (KD) and overexpression (OX) inducer titration. CRISPRi knockdowns (KD) were induced in strains carrying pCRJ001 vectors using 0.02, 0.1, and 0.2% (w/v) L-rhamnose (FIG. 10A) and overexpression from pBAD vectors induced using 0.002, 0.01, 0.02, and 0.2% (w/v) L-arabinose (FIG. 10B). Bars reflects efficiency of plating (EOP) from 30 min T7 infections (MOI=0.0001). Data represented as mean±SD (n=3). Ordinary One-way ANOVAs were performed indicating significant differences in EOP (p<0.05). A non-targeting control (NT-control) was included for KD strains and pBAD-sfGFP for OX strains.

FIG. 11A-11B: Rapid T7 enumeration assay. T7 infected log-phase BL21 carrying pJl1-T7-Pr-sfGFP. T7 RNA polymerase expressed during T7 infection drives sfGFP expression from the plasmid. Timing of sfGFP expression was dependent on T7 MOI (FIG. 11A) measured by normalized relative fluorescence units (RFU). A standard curve of log phage titer vs time to maximum sfGFP expression rate (T_{Vmax}) (FIG. 11B) was generated and used to rapidly estimate T7 titers in cell-free bacteriophage synthesis experiments to inform dilutions for plaque assays. Dotted lines represent 95% CI for simple linear regression.

FIG. 12: T7 engineering primers and DNA oligos used in the present disclosure.

FIG. 13: Gibson Assembly of synthetic phage T7 expression heterologous genes. 0.2 pmol of each of T7 genome fragments A, B, E, F, and D mixed with 0.6 pmol pelB-ClyF (left) or sfGFP (right) expression cassettes into NEBuilder® Hifi DNA Assembly mix from New England Biolabs (Ipswich, MA) to assemble synthetic T7 genomes. 1 μl of assembly mixture was used in 9 μL CFBS reaction mixture to reboot into functional phage.

FIGS. 14A-14C: Spot test of heterologous gene expression using engineered T7. T7_ClyF lysates were prepared by infecting E. coli BL21 with phage rebooted via CFBS using synthetic genomes. Lysates were spotted onto LB agar plates overlaid with 100 μL log-phase bacteria (OD=0.3)+4 mL SM agarose (50 mM TrisCl pH 7.5, 100 mM NaCl, 8 mM MgSO4, 0.3% (w/v) agarose. 3 μL of lysates were spotted on (FIG. 14A) Methicillin-resistant S. aureus (MRSA), (FIG. 14B) BL21, and (FIG. 14C) a MRSA/BL21 co-culture. Top rows show T7_ClyF lysates clearing bacteria in all 3 cases. Middle row was spotted with T7_ColE1 lysates (ColE1 is bacteriocin that kills certain strains of Enterobacteriales and not Gram-positive bacteria such as MRSA). T7_ColE1 lysates have no effect on MRSA alone, but clear BL21. T7_ColE1 creates a halo of reduced bacterial growth on MRSA/BL21, but the center of the spot is turbid with MRSA. T7(+) was included as a control on the third row with no effect on MRSA alone and similar effects to those observed in the T7_ColE1 spots.

FIG. 15: RT-qPCR primers used in the present disclosure.

FIG. 16: Vectors used in the present disclosure.

FIG. 17: overexpression sequences used in the present disclosure.

FIG. 18: sgRNA sequences used in the present disclosure.

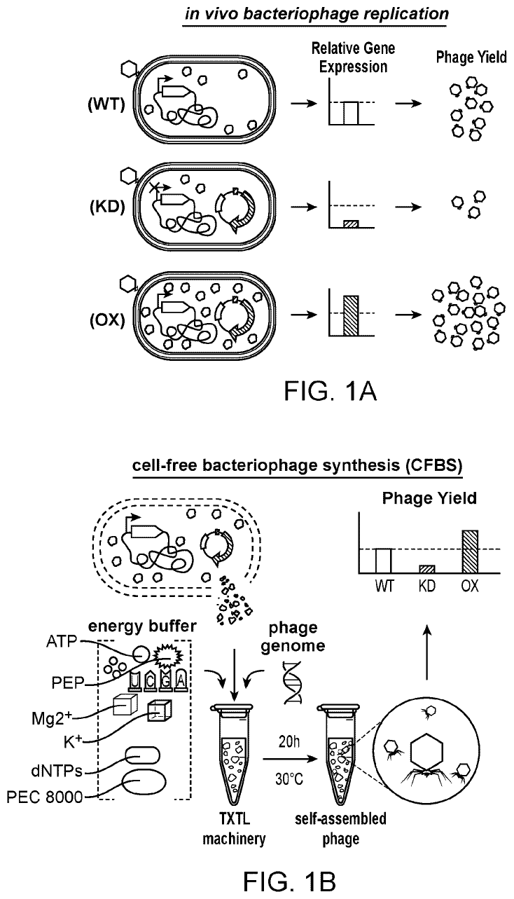

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

Bacteriophage research has been boosted by a rising interest in using phage therapy to treat antibiotic-resistant bacterial infections. In addition, there is a desire to use phages and their unique proteins for specific biocontrol applications and diagnostics. Success in the bacteriophage industry requires development of robust scalable manufacturing platforms for upstream production of high phage titres and their downstream purification and concentration whilst achieving processing yields en route. Malik D. Current Opinion in Biotechnology 68:262-271 (2021). Phage therapy is encumbered by significant challenges in the manufacturing process. Malik and Resch, Front Microbiol. 11: 584137 (2020).

The present disclosure demonstrates that lysates obtained from genetically modified bacterial host cells (e.g., E. coli) that overexpress one or more of translation initiation factor IF-3 (infC), OxyS and CyaR and/or repress RecC subunit exonuclease RecBCD achieve enhanced CFBS yields relative to conventional cell-free phage synthesis methodologies.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The term “amino acid” refers to naturally occurring and non-naturally occurring amino acids, as well as amino acid analogs and amino acid mimetics that function in a manner similar to the naturally occurring amino acids. Naturally encoded amino acids are the 20 canonical amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide is in the D form and a second plurality is in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, “bacteriophage” or “phage” refers to a virus that infects bacteria. Bacteriophages are obligate intracellular parasites that multiply inside bacteria by co-opting some or all of the host biosynthetic machinery (i.e., viruses that infect bacteria). Though different bacteriophages may contain different materials, they all contain nucleic acid and protein, and can under certain circumstances be encapsulated in a lipid membrane. Depending upon the phage, the nucleic acid can be either DNA or RNA (but not both) and can exist in various forms.

The term “CRISPR enzyme system” refers to a programmable nuclease system for genetic editing that includes a CRISPR enzyme protein (e.g., Cas12a), or derivative thereof, and one or more non-coding guide RNAs (“gRNAs”) that provide the function of a CRISPR RNA (crRNA) and trans-activating RNA (tracrRNA) for the Cas9. The crRNA and tracrRNA can be separate RNA molecules or can be combined into a single RNA molecule to produce a “single guide RNA” (sgRNA). The crRNA or the cRNA portion of the sgRNA provide sequence that is complementary to the genomic target.

As used herein, the term “effective amount” refers to a quantity sufficient to achieve a desired effect or outcome, e.g., an amount which results in the manufacturing or synthesis of bacteriophage under in vitro cell-free conditions, e.g., cell-free bacteriophage synthesis (CFBS).

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, an “expression control sequence” refers to polynucleotide sequences which are necessary to affect the expression of coding sequences to which they are operably linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to encompass, at a minimum, any component whose presence is essential for expression, and can also encompass an additional component whose presence is advantageous, for example, leader sequences.

As used herein, “heterologous nucleic acid sequence” is any sequence placed at a location in the genome where it does not normally occur. A heterologous nucleic acid sequence may comprise a sequence that does not naturally occur in a bacteriophage, or it may comprise only sequences naturally found in the bacteriophage, but placed at a non-normally occurring location in the genome. In some embodiments, the heterologous nucleic acid sequence is not a natural phage sequence. In certain embodiments, the heterologous nucleic acid sequence is a natural phage sequence that is derived from a different phage. In other embodiments, the heterologous nucleic acid sequence is a sequence that occurs naturally in the genome of a wild-type phage but is then relocated to another site where it does not naturally occur, rendering it a heterologous sequence at that new site.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same nucleobase or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

As used herein, a “host cell” is a bacterial cell that can be infected by a phage to yield progeny phage particles. A host cell can form phage particles from a particular type of phage genomic DNA. In some embodiments, the phage genomic DNA is introduced into the host cell by infecting the host cell with a phage. In some embodiments, the phage genomic DNA is introduced into the host cell using transformation, electroporation, or any other suitable technique. In some embodiments, the phage genomic DNA is substantially pure when introduced into the host cell. In some embodiments, the phage genomic DNA is present in a vector when introduced into the host cell. The definition of host cell can vary from one phage to another. For example, E. coli may be the natural host cell for a particular type of phage, but Klebsiella pneumoniae is not.

As used herein, the term “isolated” refers to a substance or entity that has been separated from at least some of the components with which it was associated when initially produced (whether in nature or in an experimental setting). Isolated substances and/or entities may be separated from at least about 10%, about 20%, about 30%, about 40%, about 50%, about 60%, about 70%, about 80%, about 90%, or more of the other components with which they were initially associated. In some embodiments, isolated substances and/or entities are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

As used herein, “operably linked” means that expression control sequences are positioned relative to the nucleic acid of interest to initiate, regulate or otherwise control transcription of the nucleic acid of interest.

As used herein, a “phage genome” includes naturally occurring phage genomes and derivatives thereof. Generally, the derivatives possess the ability to propagate in the same hosts as the naturally occurring phage. In some embodiments, the only difference between a naturally occurring phage genome and a derivative phage genome is at least one of a deletion and an addition of nucleotides from at least one end of the phage genome (if the genome is linear) or at least one point in the genome (if the genome is circular).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, for example, recombinant cells express genes that are not found within the native (non-recombinant) form of the cell or express native genes that are otherwise abnormally expressed, under expressed or not expressed at all.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous to the organism (originating from the same organism or progeny thereof) or exogenous (originating from a different organism or progeny thereof). By way of example, a promoter sequence can be substituted (e.g., by homologous recombination) for the native promoter of a gene in the genome of an organism, such that this gene has an altered expression pattern. This gene would be “recombinant” because it is separated from at least some of the sequences that naturally flank it. A nucleic acid is also considered “recombinant” if it contains any modifications that do not naturally occur in the corresponding nucleic acid in a genome. For instance, an endogenous coding sequence is considered “recombinant” if it contains an insertion, deletion or a point mutation introduced artificially, e.g., by human intervention. A “recombinant nucleic acid” also includes a nucleic acid integrated into a host cell chromosome at a heterologous site and a nucleic acid construct present as an episome.

As used herein, a “recombinant bacteriophage genome” is a bacteriophage genome that has been genetically modified by the insertion of a heterologous nucleic acid sequence into the bacteriophage genome. A “recombinant bacteriophage” means a bacteriophage that comprises a recombinant bacteriophage genome. In some embodiments, the bacteriophage genome is modified by recombinant DNA technology to introduce a heterologous nucleic acid sequence into the genome at a defined site. In some embodiments, the heterologous nucleic acid sequence is introduced with no corresponding loss of endogenous phage genomic nucleotides. In other words, if bases N1 and N2 are adjacent in the wild-type bacteriophage genome, the heterologous nucleic acid sequence is inserted between N1 and N2. Thus, in the resulting recombinant bacteriophage genome, the heterologous nucleic acid sequence is flanked by nucleotides N1 and N2. In some embodiments, endogenous phage nucleotides are removed or replaced during the insertion of the heterologous nucleic acid sequence. For example, in some embodiments, the heterologous nucleic acid sequence is inserted in place of some or all of the endogenous phage sequence which is removed. In some embodiments, endogenous phage sequences are removed from a position in the phage genome distant from the site(s) of insertion of the heterologous nucleic acid sequences.

As used herein, the term “sample” refers to clinical samples obtained from a subject or isolated microorganisms. In certain embodiments, a sample is obtained from a biological source (i.e., a “biological sample”), such as tissue, bodily fluid, or microorganisms collected from a subject. Sample sources include, but are not limited to, mucus, sputum, bronchial alveolar lavage (BAL), bronchial wash (BW), whole blood, bodily fluids, cerebrospinal fluid (CSF), urine, plasma, serum, or tissue.

### Bacteriophage

Bacteriophage are obligate intracellular parasites that multiply inside bacteria by co-opting some or all of the host biosynthetic machinery. Phages contain nucleic acid and protein, and may be enveloped by a lipid membrane. Depending upon the phage, the nucleic acid genome can be either DNA or RNA but not both, and can exist in either circular or linear forms. The size of the phage genome varies depending upon the phage. The simplest phages have genomes that are only a few thousand nucleotides in size, while the more complex phages may contain more than 100,000 nucleotides in their genome, and in rare instances no more than 500,000 bp. The number and amount of individual types of protein in phage particles will vary depending upon the phage. The proteins function in infection and to protect the nucleic acid genome from environmental nucleases.

Phage genomes come in a variety of sizes and shapes (e.g., linear or circular). Most phages range in size from 24-200 nm in diameter. The capsid is composed of many copies of one or more phage proteins, and acts as a protective envelope around the phage genome. Many phages have tails attached to the phage capsid. The tail is a hollow tube through which the phage nucleic acid passes during infection. The size of the tail can vary and some phages do not even have a tail structure. In the more complex phages, the tail is surrounded by a contractile sheath which contracts during infection of the bacterial host cell. At the end of the tail, phages have a base plate and one or more tail fibers attached to it. The base plate and tail fibers are involved in the binding of the phage to the host cell.

Lytic or virulent phages are phages which can only multiply in bacteria and lyse the bacterial host cell at the end of the life cycle of the phage. The lifecycle of a lytic phage begins with an eclipse period. During the eclipse phase, no infectious phage particles can be found either inside or outside the host cell. The phage nucleic acid takes over the host biosynthetic machinery and phage specific mRNAs and proteins are produced. Early phage mRNAs code for early proteins that are needed for phage DNA synthesis and for shutting off host DNA, RNA and protein biosynthesis. In some cases, the early proteins actually degrade the host chromosome. After phage DNA is made late mRNAs and late proteins are made. The late proteins are the structural proteins that comprise the phage as well as the proteins needed for lysis of the bacterial cell. In the next phase, the phage nucleic acid and structural proteins are assembled and infectious phage particles accumulate within the cell. The bacteria begin to lyse due to the accumulation of the phage lysis protein, leading to the release of intracellular phage particles. The number of particles released per infected cell can be as high as 1000 or more. Lytic phage may be enumerated by a plaque assay. The assay is performed at a low enough concentration of phage such that each plaque arises from a single infectious phage. The infectious particle that gives rise to a plaque is called a PFU (plaque forming unit).

Lysogenic phages are those that can either multiply via the lytic cycle or enter a quiescent state in the host cell. In the quiescent state, the phage genome exists as a prophage (i.e., it has the potential to produce phage). In most cases, the phage DNA actually integrates into the host chromosome and is replicated along with the host chromosome and passed on to the daughter cells. The host cell harboring a prophage is not adversely affected by the presence of the prophage and the lysogenic state may persist indefinitely. The lysogenic state can be terminated upon exposure to adverse conditions. Conditions which favor the termination of the lysogenic state include: desiccation, exposure to UV or ionizing radiation, exposure to mutagenic chemicals, etc. Adverse conditions lead to the production of proteases (rec A protein), the expression of the phage genes, reversal of the integration process, and lytic multiplication.

In some embodiments, a phage genome comprises at least 5 kilobases (kb), at least 10 kb, at least 15 kb, at least 20 kb, at least 25 kb, at least 30 kb, at least 35 kb, at least 40 kb, at least 45 kb, at least 50 kb, at least 55 kb, at least 60 kb, at least 65 kb, at least 70 kb, at least 75 kb, at least 80 kb, at least 85 kb, at least 90 kb, at least 95 kb, at least 100 kb, at least 105 kb, at least 110 kb, at least 115 kb, at least 120 kb, at least 125 kb, at least 130 kb, at least 135 kb, at least 140 kb, at least 145 kb, at least 150 kb, at least 175 kb, at least 200 kb, at least 225 kb, at least 250 kb, at least 275 kb, at least 300 kb, at least 325 kb, at least 350 kb, at least 375 kb, at least 400 kb, at least 425 kb, at least 450 kb, at least 475 kb, or at least 500 kb of nucleic acids.

### Cell-Free Bacteriophage Synthesis Compositions and Methods of the Present Technology

In one aspect, the present disclosure provides genetically modified bacterial host cells (e.g., E. coli) that overexpress one or more of translation initiation factor IF-3 (infC), OxyS and CyaR and/or comprise a vector that includes a nucleic acid sequence encoding a CRISPR enzyme and a vector that includes a nucleic acid sequence encoding a crRNA that specifically targets a promoter region located upstream of a transcription start site (TSS) of recC or a recC coding region located downstream of a transcription start site (TSS) of recC. In some embodiments, the vector including the nucleic acid sequence encoding the CRISPR enzyme and the vector including the nucleic acid sequence encoding the cRNA are the same or distinct.

An exemplary nucleic acid sequence of translation initiation factor IF-3 (infC) is set forth in SEQ ID NO: 39, as provided below:

(SEQ ID NO: 39)
ATTAAAGGCGGAAAACGAGTTCAAACGGCGCGCCCTAACCGTATCAATG
GCGAAATTCGCGCCCAGGAAGTTCGCTTAACAGGTCTGGAAGGCGAGCA
GCTTGGTATTGTGAGTCTGAGAGAAGCTCTGGAGAAAGCAGAAGAAGCC
GGAGTAGACTTAGTCGAGATCAGCCCTAACGCCGAGCCGCCGGTTTGTC
GTATAATGGATTACGGCAAATTCCTCTATGAAAAGAGCAAGTCTTCTAA
GGAACAGAAGAAAAAGCAAAAAGTTATCCAGGTTAAGGAAATTAAATTC
CGTCCTGGTACAGATGAAGGCGACTATCAGGTAAAACTCCGCAGCCTGA
TTCGCTTTCTCGAAGAGGGTGATAAAGCCAAAATCACGCTGCGTTTCCG
CGGTCGTGAGATGGCGCACCAGCAAATCGGTATGGAAGTGCTTAATCGC
GTGAAAGACGATTTGCAAGAACTGGCAGTGGTCGAATCCTTCCCAACGA
AGATCGAAGGCCGCCAGATGATCATGGTGCTCGCTCCTAAGAAGAAACA
GTAA

An exemplary nucleic acid sequence of OxyS is set forth in SEQ ID NO: 41, as provided below:

(SEQ ID NO: 41)
ATTCTGACTGATAATTGCTCACAGAAACGGAGCGGCACCTCTTTTAACC
CTTGAAGTCACTGCCCGTTTCGAGAGTTTCTCAACTCGAATAACTAAAG
CCAACGTGAACTTTTGCGGATCTCCAGGATCCGCTTTTTTTTGCCATAA
AAA

An exemplary nucleic acid sequence of CyaR is set forth in SEQ ID NO: 42, as provided below:

(SEQ ID NO: 42)
GCTGAAAAACATAACCCATAAAATGCTAGCTGTACCAGGAACCACCTCC
TTAGCCTGTGTAATCTCCCTTACACGGGCTTATTTTTT

In some embodiments, the genetically modified bacterial host cells comprise a non-endogenous expression vector that comprises a translation initiation factor IF-3 (infC) nucleic acid sequence of SEQ ID NO: 39, an OxyS nucleic acid sequence of SEQ ID NO: 41, a CyaR nucleic acid sequence of SEQ ID NO: 42, or any combination thereof. In some embodiments, the genetically modified bacterial host cells comprise a heterologous nucleic acid sequence that is at least 80%, 85%, 90%, 95%, 96%, 97%, 98%, or 99% identical to SEQ ID NO: 39, SEQ ID NO: 41 or SEQ ID NO: 42. Additionally or alternatively, in some embodiments, the expression levels and/or activity of infC, OxyS or CyaR in the genetically modified bacterial host cell is at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1000 times higher compared to that observed in a native bacterial host cell, wherein the genetically modified bacterial host cell is the same species as the native bacterial host cell.

Additionally or alternatively, in some embodiments, the non-endogenous expression vector comprising the infC nucleic acid sequence and the non-endogenous expression vector comprising the OxyS nucleic acid sequence and/or the CyaR nucleic acid sequence are the same or distinct. In other embodiments, the non-endogenous expression vector comprising the OxyS nucleic acid sequence and the non-endogenous expression vector comprising the CyaR nucleic acid sequence are the same or distinct.

In any of the preceding embodiments of the genetically modified bacterial host cells disclosed herein, the non-endogenous expression vector comprising the infC nucleic acid sequence, and/or the non-endogenous expression vector comprising the OxyS nucleic acid sequence, and/or the non-endogenous expression vector comprising the CyaR nucleic acid sequence is a plasmid, a cosmid, a bacmid, a bacterial artificial chromosome (BAC), or a viral vector. In certain embodiments, the non-endogenous expression vector comprising the infC nucleic acid sequence, and/or the non-endogenous expression vector comprising the OxyS nucleic acid sequence, and/or the non-endogenous expression vector comprising the CyaR nucleic acid sequence is operably linked to an expression control sequence. The expression control sequence may be an inducible promoter, a constitutive promoter, an endogenous promoter, or a heterologous promoter.

Additionally or alternatively, in some embodiments, the genetically modified bacterial host cells of the present technology comprise a vector that includes a nucleic acid sequence encoding a CRISPR enzyme and a vector that includes a nucleic acid sequence encoding a crRNA that specifically targets a promoter region located upstream of a transcription start site (TSS) of recC or a recC coding region located downstream of a transcription start site (TSS) of recC. In some embodiments, the vector including the nucleic acid sequence encoding the CRISPR enzyme and the vector including the nucleic acid sequence encoding the cRNA are the same or distinct. In certain embodiments, the nucleic acid sequence encoding the CRISPR enzyme is operably linked to an inducible promoter and/or the nucleic acid sequence encoding the crRNA is operably linked to a constitutive promoter.

Additionally or alternatively, in certain embodiments, the CRISPR enzyme is a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is any Cas12a or Cas9 protein, for instance any naturally-occurring bacterial Cas12a or Cas9 as well as any variants, homologs or orthologs thereof. Non-limiting examples of Cas proteins include Cas1, Cas1B, Cas2, Cas3, Cas4, Cas5, Cas6, Cas7, Cas8, Cas9 (also known as Csn1 and Csx12), Cas10, Cas12a from F. novicida (dFnCas12a), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or variants thereof. In some embodiments, the CRISPR enzyme cleaves both strands of the target nucleic acid at the Protospacer Adjacent Motif (PAM) site. In certain embodiments of the genetically modified bacterial host cells of the present technology, the CRISPR enzyme is a nuclease-deficient Francisella novicida Cas12a (dFnCas12a) and/or the nucleic acid sequence encoding the crRNA is selected from the group consisting of SEQ ID NO: 86, SEQ ID NO: 87, SEQ ID NO: 88 and SEQ ID NO: 89.

Additionally or alternatively, in some embodiments, the genetically modified bacterial host cells of the present technology comprise a deletion or mutation in (a) a promoter region located upstream of a transcription start site (TSS) of recC. or (b) a recC coding region located downstream of a transcription start site (TSS) of recC, wherein the mutation or deletion represses transcription of recC. The deletion may be located upstream or downstream of the transcription start site (TSS) of recC. In some embodiments, the deletion is about 10-about 100 base pairs (bps) in length. In certain embodiments, the deletion is about 10 bps, about 11 bps, about 12 bps, about 13 bps, about 14 bps, about 15 bps, about 16 bps, about 17 bps, about 18 bps, about 19 bps, about 20 bps, about 21 bps, about 22 bps, about 23 bps, about 24 bps, about 25 bps, about 26 bps, about 27 bps, about 28 bps, about 29 bps, about 30 bps, about 31 bps, about 32 bps, about 33 bps, about 34 bps, about 35 bps, about 36 bps, about 37 bps, about 38 bps, about 39 bps, about 40 bps, about 41 bps, about 42 bps, about 43 bps, about 44 bps, about 45 bps, about 46 bps, about 47 bps, about 48 bps, about 49 bps, about 50 bps, about 51 bps, about 52 bps, about 53 bps, about 54 bps, about 55 bps, about 56 bps, about 57 bps, about 58 bps, about 59 bps, about 60 bps, about 61 bps, about 62 bps, about 63 bps, about 64 bps, about 65 bps, about 66 bps, about 67 bps, about 68 bps, about 69 bps, about 70 bps, about 71 bps, about 72 bps, about 73 bps, about 74 bps, about 75 bps, about 76 bps, about 77 bps, about 78 bps, about 79 bps, about 80 bps, about 81 bps, about 82 bps, about 83 bps, about 84 bps, about 85 bps, about 86 bps, about 87 bps, about 88 bps, about 89 bps, about 90 bps, about 91 bps, about 92 bps, about 93 bps, about 94 bps, about 95 bps, about 96 bps, about 97 bps, about 98 bps, about 99 bps, or about 100 bps in length. In other embodiments, the mutation is a nonsense mutation, a frameshift mutation, or a missense mutation.

In any of the foregoing embodiments described herein, the genetically modified bacterial host cells are E. coli cells.

In one aspect, the present disclosure provides a cell lysate obtained from any and all embodiments of the genetically modified bacterial host cells disclosed herein. The cell lysates of the present technology comprise an effective amount of transcription/translation (TXTL) machinery that is configured to synthesize bacteriophage under cell-free conditions. The cell lysates may be prepared using one or more of French-press cell lysis, sonication, runoff reactions, or lysate dialysis.

In another aspect, the present disclosure provides an in vitro method for synthesizing bacteriophage virions comprising contacting a bacteriophage genome with any and all embodiments of the cell lysates disclosed herein and an energy buffer in vitro to obtain a reaction mixture, and incubating the reaction mixture under conditions to produce viable phage virions, wherein the energy buffer comprises canonical amino acids, phosphoenol pyruvate (PEP), nucleoside triphosphates (NTPs), cofactors, and coenzymes. In some embodiments, the reaction mixture is incubated at 16° C. to 42° C. for about 1-24 hours. In certain embodiments, the reaction mixture is incubated at 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., or 42° C. for about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. The bacteriophage genome may be derived from a naturally occurring bacteriophage, or a genetically engineered bacteriophage. In some embodiments, the bacteriophage genome is obtained from a bacteriophage that specifically infects the naturally occurring counterpart of the genetically modified bacterial host cells disclosed herein, such as E. coli. Additionally or alternatively, in some embodiments, the bacteriophage genome is obtained from a bacteriophage selected from among T7 phage, T7-like phage, Lambda, K1E, T3, T5, T4, or PhiX174.

Additionally or alternatively, in some embodiments, the energy buffer further comprises one or more of a polyethylene glycol (PEG) polymer (such as PEG-8000 or PEG-6000), deoxynucleotide triphosphates (dNTPs), and stabilizers. In certain embodiments, the energy buffer further comprises one or more of Mg-glutamate, K-glutamate, tRNA, cAMP, folinic acid, spermidine, 3-PGA, HEPES, Nicotinamide adenine dinucleotide (NAD), coenzyme A (CoA), DTT, and maltodextrin.

In any of the preceding embodiments of the methods disclosed herein, the reaction mixture further comprises a vector including a nucleic acid sequence encoding a reporter gene, wherein the nucleic acid sequence encoding the reporter gene is operably linked to a promoter that is responsive to a RNA polymerase (RNAP) encoded by the bacteriophage genome. In certain embodiments, the RNAP is selected from among SP6 RNA Polymerase, T3 RNA Polymerase and T7 RNA polymerase. The reporter gene may encode a bioluminescent protein, a fluorescent protein, or a chemiluminescent protein. Examples of bioluminescent proteins include, but are not limited to, Aequorin, firefly luciferase, Renilla luciferase, red luciferase, luxAB, or nanoluciferase. Examples of chemiluminescent proteins include, but are not limited to, β-galactosidase, horseradish peroxidase (HRP), or alkaline phosphatase. Examples of fluorescent proteins include, but are not limited to, sfGFP, TagBFP, Azurite, EBFP2, mKalamal, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOx, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherryl, PATagRFP, Kaede (green), Kaede (red), KikGR1 (green), KikGR1 (red), PS-CFP2, PS-CFP2, mEos2 (green), mEos2 (red), PSmOrange, or Dronpa.

Additionally or alternatively, in some embodiments of the methods disclosed herein, the phage virions show increased efficiency of plating (EOP) relative to phage virions obtained with cell-free bacteriophage synthesis (CFBS) in a control lysate obtained from a wild-type bacterial cell, wherein the wild-type bacterial cell and the genetically modified bacterial host cell are the same species.

### Polynucleotides, Polypeptides and Analogs

Also included in the presently disclosed subject matter are infC, OxyS, CyaR and/or RecC cRNA polynucleotides and their corresponding polypeptides or fragments that may be modified in ways that enhance their functional activity when expressed in a bacterial host cell. The presently disclosed subject matter provides methods for optimizing an amino acid sequence or a nucleic acid sequence by producing an alteration in the sequence. Such alterations can comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences. Analogs of the presently disclosed subject matter can generally exhibit at least about 85%, about 90%, about 91%, about 92%, about 93%, about 94%, about 95%, about 96%, about 97%), about 98%, about 99% or more identity or homology with all or part of a naturally-occurring amino, acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program can be used, with a probability score between e⁻³ and e⁻¹⁰⁰ indicating a closely related sequence.

Analogs can differ from the naturally-occurring polypeptides of the presently disclosed subject matter by alterations in primary sequence. These include genetic variants, both natural and induced (for example, resulting from random mutagenesis by irradiation or exposure to ethanemethyl sulfate or by site-specific mutagenesis as described in Sambrook, Fritsch and Maniatis, Molecular Cloning: A Laboratory Manual (2nd ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids.

In accordance with the presently disclosed subject matter, the polynucleotides encoding infC, OxyS, CyaR and/or RecC cRNA can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

### Vectors

As used herein, a “vector” is a replicable nucleic acid from which one or more heterologous proteins or RNAs can be expressed when the vector is introduced into an appropriate host cell. The vector is used to introduce the nucleic acid encoding the polypeptide or RNA into the host cell for amplification of the nucleic acid or for expression/display of the polypeptide or RNA encoded by the nucleic acid. As used herein, a vector also includes “virus vectors” or “viral vectors.” Viral vectors are engineered viruses that are operatively linked to exogenous genes to transfer (as vehicles or shuttles) the exogenous genes into cells.

Many expression vectors are available and known to those of skill in the art and can be used for nonendogenous expression of infC, OxyS, CyaR, and/or a RecC cRNA. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art.

Vectors also can contain additional nucleotide sequences operably linked to the ligated nucleic acid molecule, such as, for example, an epitope tag such as for localization, e.g. a hexa-his tag or a myc tag, hemagglutinin tag or a tag for purification, for example, a GST fusion, and a sequence for directing protein secretion and/or membrane association.

Expression of the infC, OxyS, or CyaR genes and/or a RecC cRNA can be controlled by any promoter/enhancer known in the art. Suitable bacterial promoters are well known in the art and described herein below. Selection of the promoter used to direct expression of a heterologous nucleic acid depends on the particular application and is within the level of skill of the skilled artisan. Promoters which can be used include but are not limited to prokaryotic expression vectors such as the β-lactamase promoter (Jay et al. (1981) Proc. Natl. Acad. Sci. USA 75:5543), the tac promoter (DeBoer et al. (1983) Proc. Natl. Acad. Sci. USA 50:21-25); see also “Useful Proteins from Recombinant Bacteria” (1980) in Scientific American 242:79-94), rhaB promoter and the like.

In addition to the promoter, the expression vector typically contains a transcription unit or expression cassette that contains all the additional elements required for the expression of the heterologous nucleic acid, in host cells. A typical expression cassette contains a promoter operably linked to the gene sequence and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Any methods known to those of skill in the art for the insertion of DNA fragments into a vector can be used to construct expression vectors containing a nucleic acid encoding any of the genes disclosed herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Genetic modification of engineered host cells can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding infC, OxyS, CyaR and/or a RecC cRNA can be cloned into a retroviral vector and expression can be driven from its endogenous promoter, from the retroviral long terminal repeat, or from an alternative internal promoter.

Non-viral vectors or RNA can be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide cells expressing infC, OxyS, or CyaR genes and/or a RecC cRNA, a retroviral vector can be employed for transduction. However, any other suitable viral vector or non-viral delivery system can be used for genetic modification of cells.

Possible methods of transduction also include direct co-culture of the cells with producer cells, e.g., by the method of Bregni et al. (1992) Blood 80: 1418-1422, or culturing with viral supernatant alone or concentrated vector stocks with or without appropriate growth factors and polycations, e.g., by the method of Xu et al. (1994) Exp. Hemat. 22:223-230; and Hughes et al. (1992) J. Clin. Invest. 89: 1817.

In some embodiments, the chosen vector exhibits high efficiency of infection and stable integration and expression (see, e.g., Cayouette et al. (1997) Human Gene Therapy 8:423-430; Kido et al. (1996) Current Eye Research 15:833-844; Bloomer et al. (1997) Journal of Virology 71:6641-6649; Naldini et al. (1996) Science 272:263 267; and Miyoshi et al. (1997) Proc. Natl. Acad. Sci. U.S.A. 94: 10319). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller (1990) Human Gene Therapy 15-14; Friedman (1989) Science 244: 1275-1281; Eglitis et al. (1988) BioTechniques 6:608-614; Tolstoshev et al. (1990) Current Opinion in Biotechnology 1:55-61; Sharp (1991) The Lancet 337: 1277-1278; Cornetta et al. (1987) Nucleic Acid Research and Molecular Biology 36:311-322; Anderson (1984) Science 226:401-409; Moen (1991) Blood Cells 17:407-416; Miller et al. (1989) Biotechnology 7:980-990; Le Gal La Salle et al. (1993) Science 259:988-990; and Johnson (1995) Chest 107:77S-83S). Retroviral vectors are particularly well developed and have been used in clinical settings (Rosenberg et al. (1990) N. Engl. J. Med 323:370; Anderson et al., U.S. Pat. No. 5,399,346).

In certain non-limiting embodiments, the vector expressing an infC, OxyS, CyaR and/or RecC cRNA nucleic acid sequence is a retroviral vector, e.g., an oncoretroviral vector. In some instances, the retroviral vector is a SFG retroviral vector or murine stem cell virus (MSCV) retroviral vector. In certain non-limiting embodiments, the vector expressing a an infC, OxyS, CyaR and/or RecC cRNA nucleic acid sequence is a lentiviral vector. In certain non-limiting embodiments, the vector expressing an infC, OxyS, CyaR and/or RecC cRNA nucleic acid sequence is a transposon vector.

Non-viral approaches can also be employed for the expression of a protein in cell. For example, a nucleic acid molecule can be introduced into a cell by administering the nucleic acid in the presence of lipofection (Feigner et al. (1987) Proc. Nat'l. Acad. Sci. U.S.A. 84:7413; Ono et al. (1990) Neuroscience Letters 17:259; Brigham et al. (1989) Am. J. Med. Sci. 298:278; Staubinger et al. (1983) Methods in Enzymology 101:512), asialoorosomucoid-polylysine conjugation (Wu et al. (1988) Journal of Biological Chemistry 263: 14621; Wu et al. (1989) Journal of Biological Chemistry 264: 16985), or by micro-injection under surgical conditions (Wolff et al. (1990) Science 247: 1465). Other non-viral means for gene transfer include transfection in vitro using calcium phosphate, DEAE dextran, electroporation, and protoplast fusion. Liposomes can also be potentially beneficial for delivery of DNA into a cell. Transient expression can be obtained by RNA electroporation.

The resulting cells can be grown under conditions similar to those for unmodified cells, whereby the modified cells can be expanded and used for a variety of purposes.

### Kits

The present technology provides kits for preparing donor bacterial host cell lysates for cell-free bacteriophage synthesis (CFBS). In one aspect, the present disclosure provides a kit comprising one or more expression vectors comprising at least one nucleic acid sequence selected from the group consisting of SEQ ID NO: 39, SEQ ID NO: 41 and SEQ ID NO: 42, and instructions for using the same to prepare donor bacterial cell lysates for cell-free bacteriophage synthesis (CFBS). In certain embodiments, the at least one nucleic acid sequence is operably linked to an inducible promoter, a constitutive promoter, an endogenous promoter, or a heterologous promoter. Additionally or alternatively, in some embodiments, the kits further comprise vials containing naturally-occurring or non-natural bacterial host cells that can be transformed with the one or more expression vectors comprising at least one nucleic acid sequence selected from the group consisting of SEQ ID NO: 39, SEQ ID NO: 41 and SEQ ID NO: 42. In some embodiments, the bacterial host cells are E. coli. In certain embodiments, the bacterial host cells are E. coli strain BL21.

In another aspect, the present disclosure provides a kit comprising one or more expression vectors comprising at least one nucleic acid sequence encoding a crRNA that specifically targets a promoter region located upstream of a transcription start site (TSS) of recC or a recC coding region located downstream of a transcription start site (TSS) of recC, and instructions for using the same to prepare donor bacterial cell lysates for cell-free bacteriophage synthesis (CFBS). In some embodiments, the one or more expression vectors further comprise a nucleic acid sequence encoding a CRISPR enzyme. Examples of CRISPR enzymes include, but are not limited to, nuclease-deficient Cas12a from F. novicida (dFnCas12a), Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4. Additionally or alternatively, in some embodiments, the nucleic acid sequence encoding the CRISPR enzyme is operably linked to an inducible promoter and/or the at least one nucleic acid sequence encoding the crRNA is operably linked to a constitutive promoter. In some embodiments, the nucleic acid sequence encoding the crRNA is selected from the group consisting of SEQ ID NO: 86, SEQ ID NO: 87, SEQ ID NO: 88 and SEQ ID NO: 89. Additionally or alternatively, in some embodiments, the kits further comprise vials containing naturally-occurring or non-natural bacterial host cells that can be transformed with the one or more expression vectors comprising the nucleic acid sequence encoding the CRISPR enzyme and/or the at least one nucleic acid sequence encoding the crRNA. In some embodiments, the bacterial host cells are E. coli. In certain embodiments, the bacterial host cells are E. coli strain BL21.

In yet another aspect, the present disclosure provides a kit comprising any and all embodiments of the genetically modified bacterial host cell described herein, and instructions for using the same to prepare donor bacterial cell lysates for cell-free bacteriophage synthesis (CFBS). In some embodiments, the genetically modified bacterial host cells are E. coli. In certain embodiments, the bacterial host cells are E. coli strain BL21.

In any and all embodiments of the kits disclosed herein, the kits further comprise one or more of polyethylene glycol (PEG) polymers (e.g., PEG-8000, PEG-6000), deoxynucleotide triphosphates (dNTPs), stabilizers, and an energy buffer comprising canonical amino acids, phosphoenol pyruvate (PEP), nucleoside triphosphates (NTPs), cofactors, and coenzymes.

Additionally or alternatively, in some embodiments, the kits further comprise a vector including a nucleic acid sequence encoding a reporter gene, wherein the nucleic acid sequence encoding the reporter gene is operably linked to a promoter that is responsive to a phage RNA polymerase (RNAP), such as SP6 RNA Polymerase, T3 RNA Polymerase and T7 RNA polymerase. In some embodiments, the reporter gene encodes a bioluminescent protein, a fluorescent protein, or a chemiluminescent protein. Examples of bioluminescent proteins include, but are not limited to, Aequorin, firefly luciferase, Renilla luciferase, red luciferase, luxAB, or nanoluciferase. Examples of chemiluminescent proteins include, but are not limited to, β-galactosidase, horseradish peroxidase (HRP), or alkaline phosphatase. Examples of fluorescent proteins include, but are not limited to, sfGFP, TagBFP, Azurite, EBFP2, mKalamal, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOx, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherryl, PATagRFP, Kaede (green), Kaede (red), KikGR1 (green), KikGR1 (red), PS-CFP2, PS-CFP2, mEos2 (green), mEos2 (red), PSmOrange, or Dronpa.

In any of the preceding embodiments, the kits of the present technology comprise one or more coded/labeled vials that contain a plurality of bacteriophage genomes. The plurality of bacteriophage genomes may be obtained from naturally occurring bacteriophage or genetically engineered bacteriophage. In some embodiments, each coded/labeled vial containing a plurality of bacteriophage genomes corresponds to a different bacteriophage type. In other embodiments, each coded/labeled vial containing a plurality of bacteriophage genomes corresponds to the same bacteriophage type. In some embodiments, each phage vial is assigned a unique code that identifies the bacteriophage in the phage vial, or the types of bacteria that the bacteriophage strain infects. The unique code can be encoded by a machine discernible pattern, such as a bar code, a QR code, an alphanumeric string, or any other pattern that can be discerned by a reader. Each unique code may be shown as, for example, a bar code sticker on a vial or container storing a corresponding phage sample. In some embodiments, the kit is stored under conditions that permit the preservation of the bacteriophage genomes for extended periods, such as under bacteriophage-specific, controlled temperature, moisture, and pH conditions.

The kit may further comprise one or more of: wash buffers and/or reagents, hybridization buffers and/or reagents, labeling buffers and/or reagents, and detection means. The buffers and/or reagents are usually optimized for the particular detection technique for which the kit is intended. Protocols for using these buffers and reagents for performing different steps of the procedure may also be included in the kit. Further optional components of the kits may include expression media for reporter gene products disclosed herein, such as a medium containing nutrients and cofactors for bioluminescence, devices such as a lamp configured to illuminate at specific wavelengths of light to detect biofluorescence, and devices for measuring the extent of expression of the reporter genes, such as a photometer or photodetector.

In some embodiments, the kits further comprise positive control heterologous nucleic acid sequences to correct for any variability in the CFBS systems between experimental runs. The kits may also comprise instructions for use, software for automated analysis, containers, packages such as packaging intended for commercial sale and the like.

### EXAMPLES

The present technology is further illustrated by the following Examples, which should not be construed as limiting in any way.

### Example 1: Materials and Methods

### Reagents

The following CFES/CFBS components, buffer salts, and media supplements were purchased from Sigma-Aldrich (St. Louis, MO): ATP, CTP, GTP, E. coli total tRNA mixture (from strain MRE600), 5-Formyl-5,6,7,8-tetrahydrofolic acid (folinic acid), 4-(2-hydroxyethyl)-1-piperazineethanesulfonic acid (HEPES), nicotinamide adenine dinucleotide (NAD), cyclic adenosine monophosphate (cAMP), coenzyme A (CoA), spermidine, 18 canonical amino acids excluding glutamate and leucine, polyethylene glycol 8000 (PEG-8000), maltodextrin, sodium oxalate, 1,4-dithiothreitol (DTT), potassium glutamate (K(glu)), magnesium glutamate (Mg(glu)₂), magnesium sulfate, calcium chloride, DNase I, lysozyme, and proteinase K. Leucine, phosphoenolpyruvate (PEP), and agar were purchased from Fisher Scientific (Hampton, NH) and UTP from Alfa Aesar (Havervill, MA). RecBCD nuclease inhibitor GamS, dNTPs, and all plasmid cloning reagents were purchased from New England Biolabs (NEB, Ipswich, MA). Tryptone, yeast extract, sodium chloride, potassium phosphate monobasic, potassium phosphate dibasic, agarose were purchased from VWR (Radnor, PA). TURBO DNA-free™ Kit and SuperScript™ II Reverse Transcriptase were purchased from Invitrogen (Waltham, MA). All statistical analysis was performed using GraphPad Prism 9.

### Bacterial Strains and Growth Conditions

E. coli DH5α [genotype F⁻Φ80lacZΔM15Δ(lacZYA-argF) U169 recA1 endA1 hsdR17(r_{K}⁻, m_{K}⁺)phoA supE44 thi-1 gyrA96 relA1 λ−] was used for plasmid cloning (NEB, Ipswich, MA). E. coli BL21 ATCC BAA-1025 [genotype F⁻ompT lon hsdS_{B}(r_{B}⁻ m_{B}⁻)gal dcm [malB⁺]_{K-12}(λ^{S})] was used for phage propagation, in vivo phage fitness and quantification assays, and as a source of cell extract for cell-free bacteriophage synthesis (CFBS) (American Tissue Culture Collection, Manassas, VA). The E. coli cells were grown at 37° C. in Luria-Bertani (LB) media (10 g/L of tryptone, 5 g/L of yeast extract, 10 g/L of sodium chloride in Milli-Q water). For T7 phage experiments, ΦLB (LB+2.5 mM MgSO₄ and 2.5 mM CaCl₂) was used. Solid media incorporated 1.500 agarose. All E. coli and phage strains used in this study are listed in Table A.

TABLE A
E. coli and phage strains

| Strain | Relevant genotype | Source |
|---|---|---|
| E. coli | F⁻Φ80lacZΔM15Δ(lacZYA-argF) | NEB C2987I |
| DH5α | U169 recAl endAl hsdR17(r_{K}⁻, | |
| | m_{K}⁺) phoA supE44 thi-1 | |
| | gyrA96 relA1 λ- | |
| E. coli BL21 | F⁻ompT lon hsdS_{B}(r_{B}⁻ m_{B}⁻) | ATCC BAA-1025 |
| | gal dcm [malB⁺]_{K-12}(λ^{S}) | |
| E. coli | N/a | ATCC |
| bacteriophage | | BAA-1025-B2 |
| T7 | | |

All plasmids used in this study are listed in Table B.

TABLE B
Plasmids

| Plasmid | Relevant features | Reference or source |
|---|---|---|
| pJL1-sfGFP | T7 promoter-T7 gene 10 RBS-sfGFP-T7 | Addgene # |
| | terminator kanR (kanamycin resistance) | 69496 |
| | ColE1 ori | |
| pBAD-sfGFP | araBAD promoter-T7gene 10 RBS-sfGFP-rrnB1 | Addgene |
| | T1 terminator ampR (ampicillin resistance) | #54519 |
| | ColE1 ori | |
| pCRJ001 | rhaB promoter-RBS-dFnCas12a (aka | This study |
| | dFnCpf1)-rrnB1 terminator; J23119 | |
| | promoter-FnCas12a sgRNA scaffold-LacZα cat | |
| | (chloramphenicol resistance) p15A ori | |
| pCRJ001-g001 | pCRJ001 derivative expressing sgRNA targeting | This study |
| | trxA promoter | |
| pCRJ001-g002 | pCRJ001 derivative expressing sgRNA targeting | This study |
| | trxA CDS | |
| pCRJ001-g003 | pCRJ001 derivative expressing sgRNA targeting | This study |
| | nusG promoter | |
| pCRJ001-g004 | pCRJ001 derivative expressing sgRNA targeting | This study |
| | nusG CDS | |
| pCRJ001-g005 | pCRJ001 derivative expressing sgRNA targeting | This study |
| | pgk promoter | |
| pCRJ001-g006 | pCRJ001 derivative expressing sgRNA targeting | This study |
| | pgk CDS | |
| pCRJ001-g007 | pCRJ001 derivative expressing sgRNA targeting | This study |
| | subH promoter | |
| pCRJ001-g008 | pCRJ001 derivative expressing sgRNA targeting | This study |
| | subH CDS | |
| pCRJ001-g009 | pCRJ001 derivative expressing sgRNA targeting | This study |
| | eno promoter | |
| pCRJ001-g010 | pCRJ001 derivative expressing sgRNA targeting | This study |
| | eno CDS | |
| pCRJ001-g011 | pCRJ001 derivative expressing sgRNA targeting | This study |
| | mukB promoter | |
| pCRJ001-g012 | pCRJ001 derivative expressing sgRNA targeting | This study |
| | mukB CDS | |
| pCRJ001-g013 | pCRJ001 derivative expressing sgRNA targeting | This study |
| | hemL promoter | |
| pCRJ001-g014 | pCRJ001 derivative expressing sgRNA targeting | This study |
| | hemL CDS | |
| pCRJ001-g015 | pCRJ001 derivative expressing sgRNA targeting | This study |
| | cyaR promoter | |
| pCRJ001-g016 | pCRJ001 derivative expressing sgRNA targeting | This study |
| | cyaR CDS | |
| pCRJ001-g017 | pCRJ001 derivative expressing sgRNA targeting | This study |
| | lexA promoter | |
| pCRJ001-g018 | pCRJ001 derivative expressing sgRNA targeting | This study |
| | lexA CDS | |
| pCRJ001-g019 | pCRJ001 derivative expressing sgRNA targeting | This study |
| | infC promoter | |
| pCRJ001-g020 | pCRJ001 derivative expressing sgRNA targeting | This study |
| | infC CDS | |
| pCRJ001-g021 | pCRJ001 derivative expressing sgRNA targeting | This study |
| | recC promoter | |
| pCRJ001-g022 | pCRJ001 derivative expressing sgRNA targeting | This study |
| | recC CDS | |
| pCRJ001-g023 | pCRJ001 derivative expressing sgRNA targeting | This study |
| | dgt promoter | |
| pCRJ001-g024 | pCRJ001 derivative expressing sgRNA targeting | This study |
| | dgt CDS | |
| pCRJ001-g025 | pCRJ001 derivative expressing sgRNA targeting | This study |
| | udk promoter | |
| pCRJ001-g026 | pCRJ001 derivative expressing sgRNA targeting | This study |
| | udk CDS | |
| pCRJ001-g027 | pCRJ001 derivative expressing sgRNA targeting | This study |
| | rna promoter | |
| pCRJ001-g028 | pCRJ001 derivative expressing sgRNA targeting | This study |
| | rna CDS | |
| pCRJ001-g030 | pCRJ001 derivative expressing sgRNA targeting | This study |
| | ΦT7 gene 3 CDS | |
| pCRJ001-g031 | pCRJ001 derivative expressing sgRNA targeting | This study |
| | oxyS promoter | |
| pCRJ001-g032 | pCRJ001 derivative expressing sgRNA targeting | This study |
| | oxyS CDS | |
| pCRJ001-g033 | pCRJ001 derivative expressing sgRNA targeting | This study |
| | rne promoter | |
| pCRJ001-g034 | pCRJ001 derivative expressing sgRNA targeting | This study |
| | rne CDS | |
| pCRJ001-g035 | pCRJ001 derivative expressing sgRNA targeting | This study |
| | trxB promoter | |
| pCRJ001-g036 | pCRJ001 derivative expressing sgRNA targeting | This study |
| | trxB CDS | |
| pCRJ001-g037 | pCRJ001 derivative expressing non-targeting | This study |
| | control sgRNA | |
| pBAD-trxA | pBAD derivative for trxA overexpression | This study |
| pBAD-trxB | pBAD derivative for trxB overexpression | This study |
| pBAD-mukB | pBAD derivative for mukB overexpression | This study |
| pBAD-infC | pBAD derivative for infC overexpression | This study |
| pBAD-rne | pBAD derivative for rne overexpression | This study |
| pBAD-oxyS | pBAD derivative for oxyS overexpression | This study |
| pBAD-cyaR | pBAD derivative for cyaR overexpression | This study |
| pBAD-dgt | pBAD derivative for dgt overexpression | This study |

sgRNA = single-guide RNA
CDS = coding sequence downstream of transcription start sites

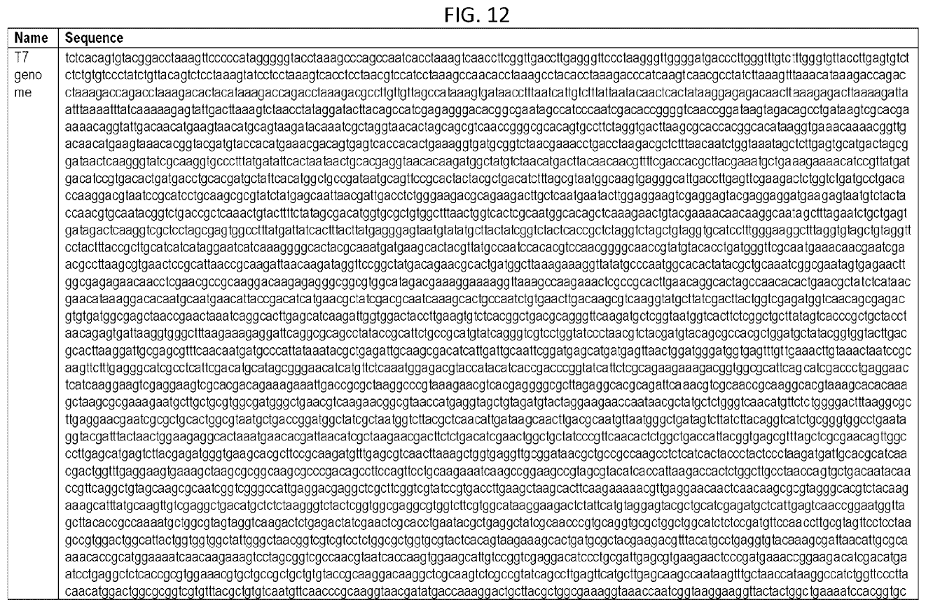

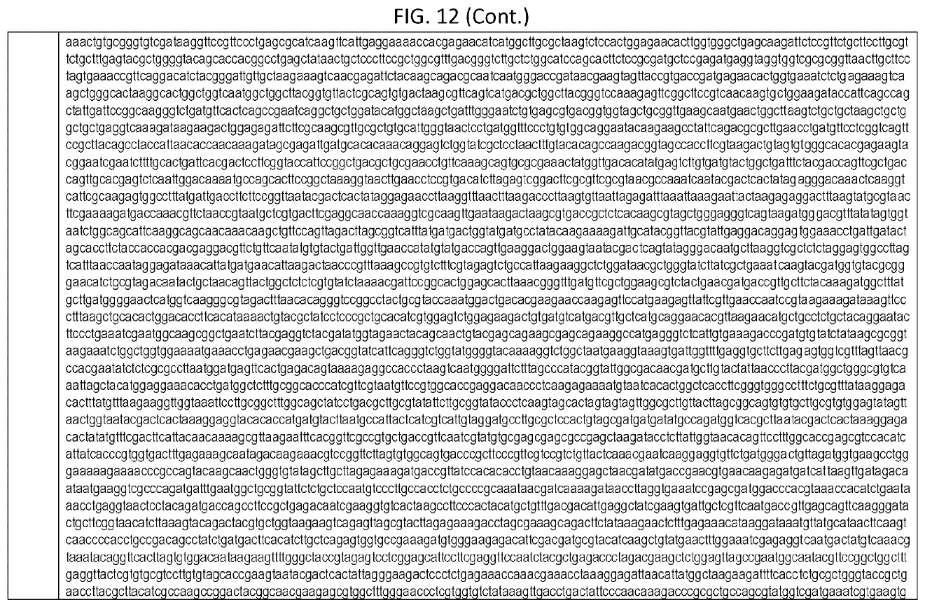

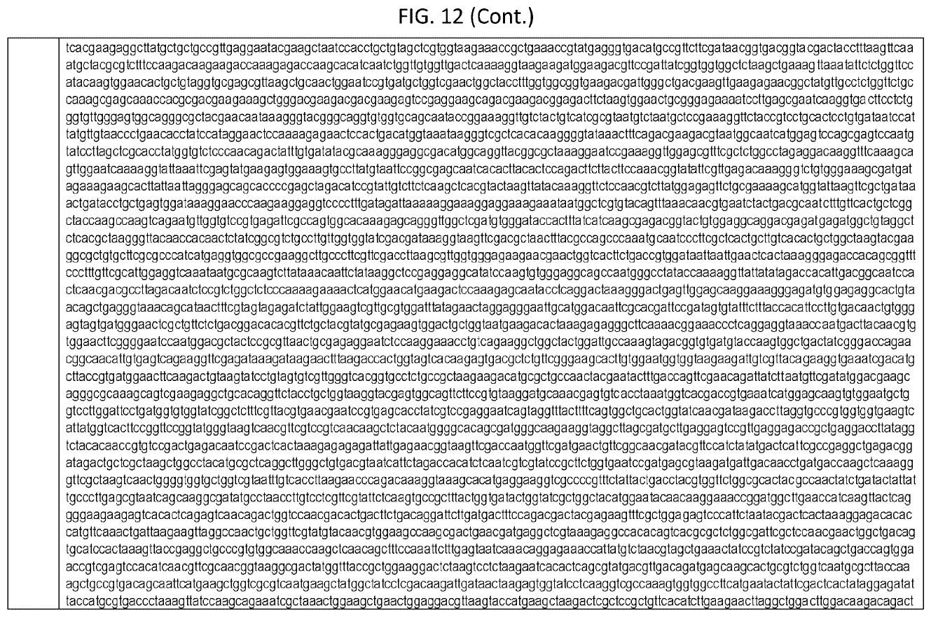

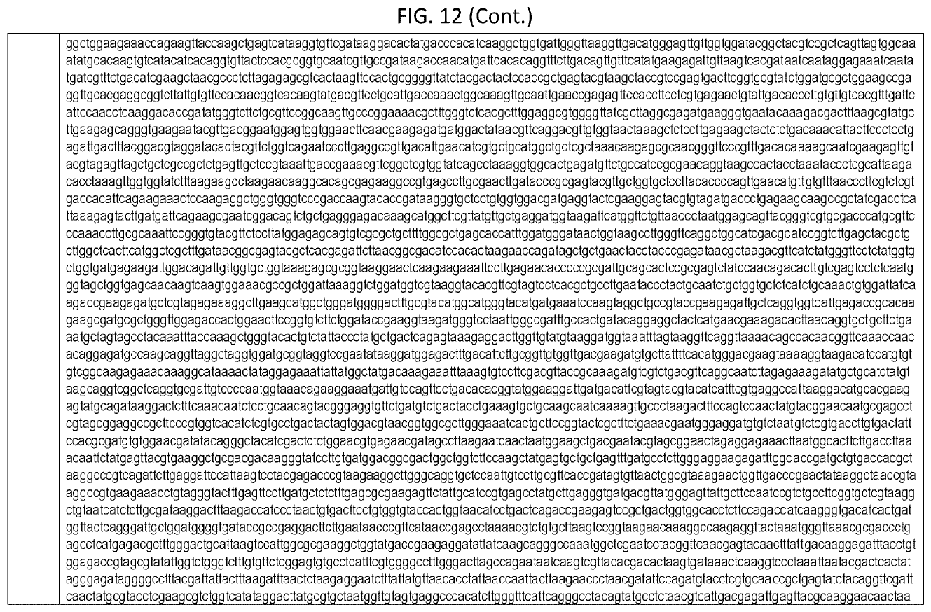

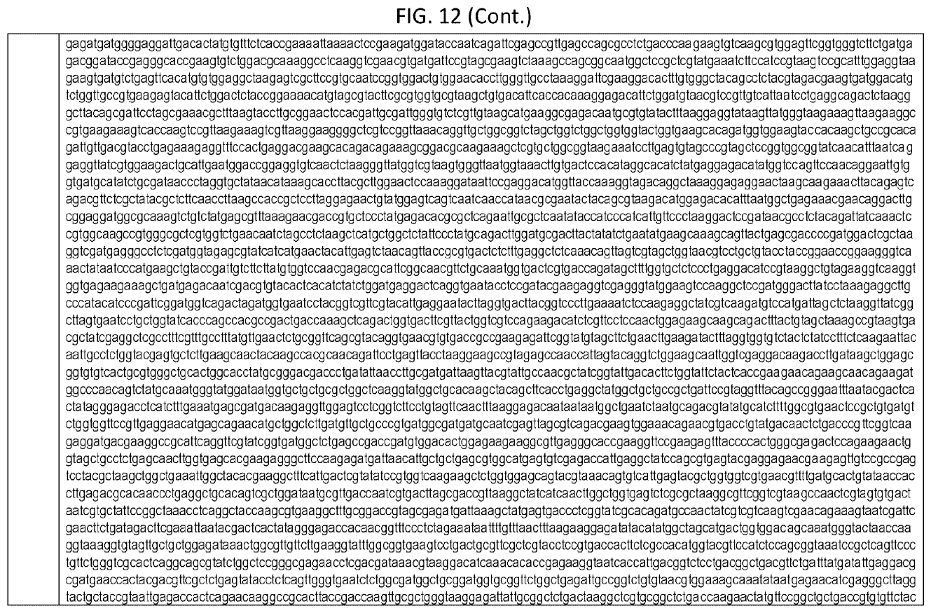

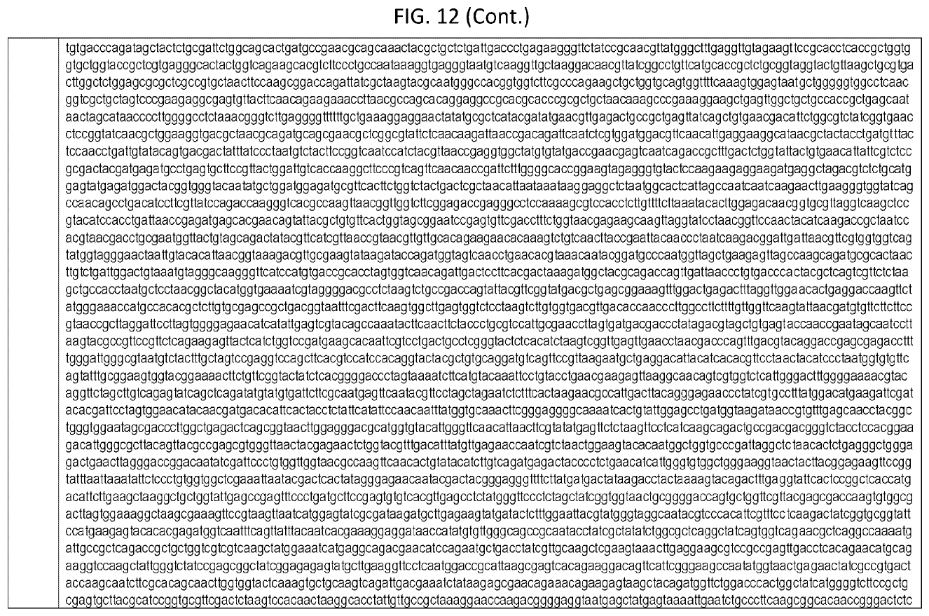

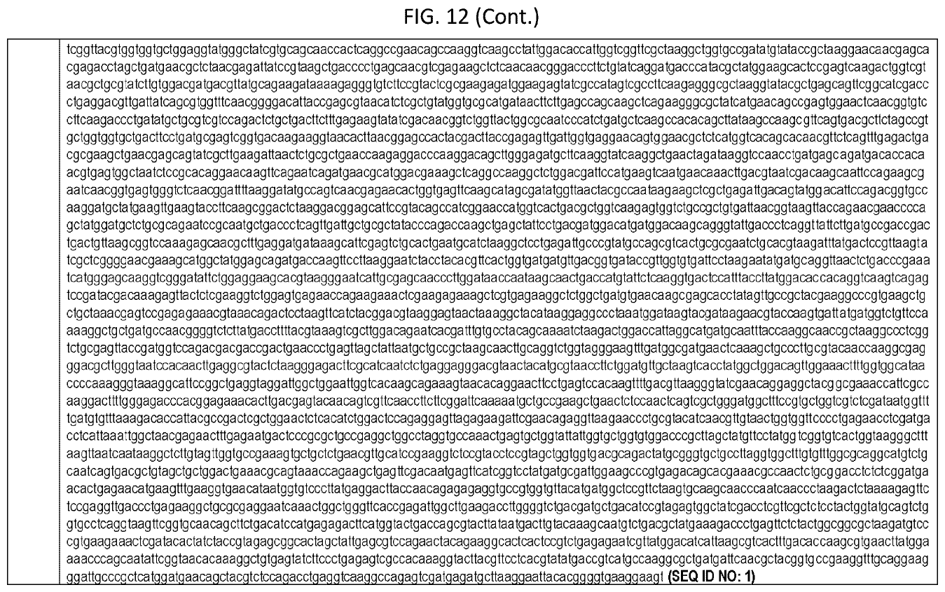

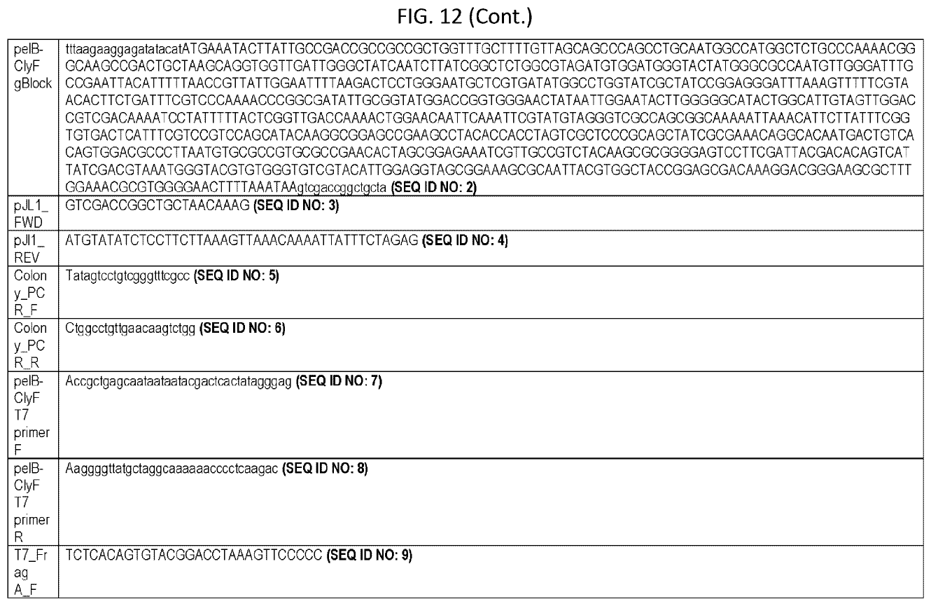

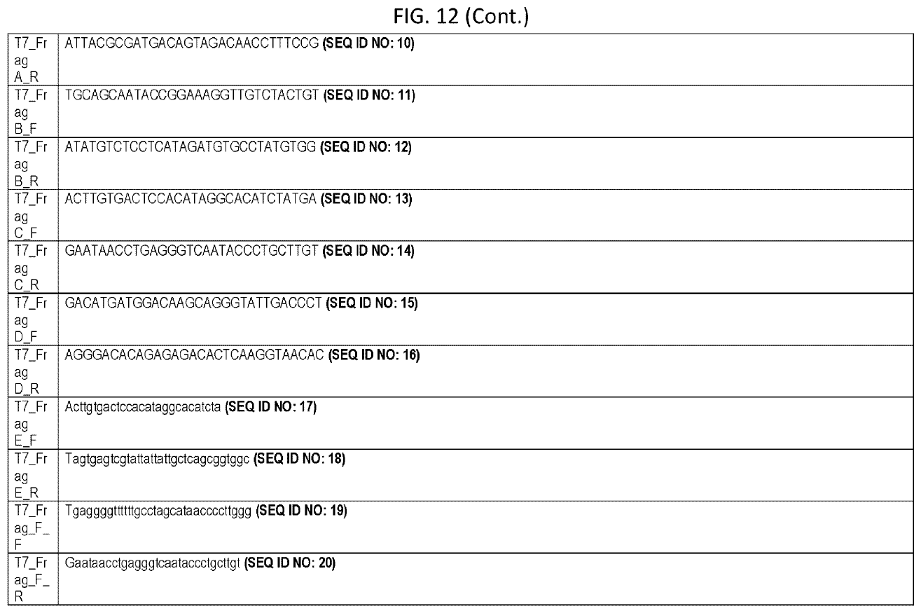

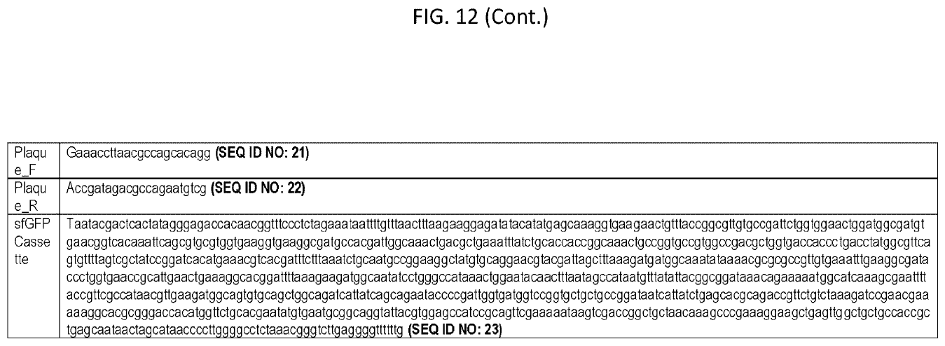

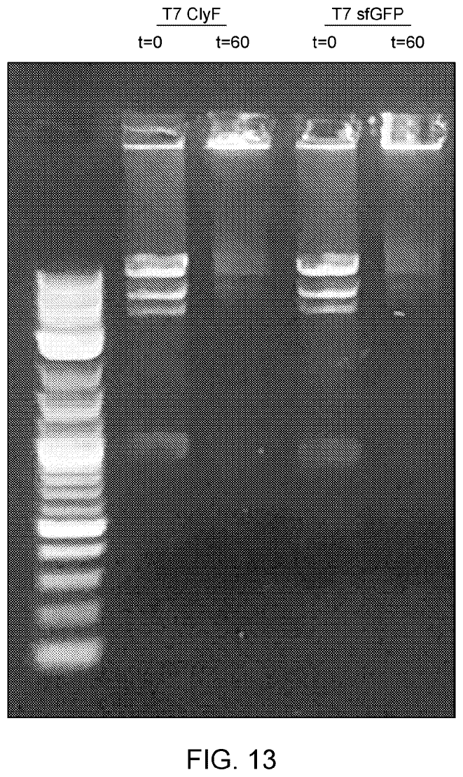

Primers and DNA oligos were purchased from integrated DNA Technologies (Coralville, IA) and are listed in FIG. 12.

### Phage Propagation

E. coli bacteriophage T7 (ATCC BAA-1025-B2) was propagated using E. coli BL21 as the host strain. BL21 was grown for overnight (16 h) in 10 mL ΦLB at 37° C. inoculated with a single colony. The overnight culture was transferred to 100 mL prewarmed ΦLB in a 500 mL Erlenmeyer flask and incubated for 1 h shaking at 250 rpm to bring the culture to log phase. This culture was then inoculated with 100 μL 10⁸ PFU/mL T7 lysate and allowed to incubate until complete bacterial lysis was visually observed. The lysate was allowed to shake incubate a further 10 min after addition of 1 mL chloroform to lyse remaining BL21 cells. The lysate was transferred to 50 mL conical tubes and centrifuged at 10,000×g for 5 min to pellet cell debris and separate chloroform from the mixture. Supernatants were recovered, 0.22 μm sterile-filtered, then placed on ice for 1 h. Phage were concentrated by PEG-precipitation (Jakociune, D.; Moodley, A., A Rapid Bacteriophage DNA Extraction Method. Methods Protoc 2018, 1 (3)). Briefly, 0.22 μm sterile-filtered PEG-8000/NaCl solution was mixed with lysate supernatants to final concentrations of 4% (w/v) and 0.5 M respectively followed by overnight incubation at 4° C. to encourage precipitation. Phage were pelleted by centrifugation at 10,000×g at 4° C. for 30 min. Supernatants were carefully decanted and discarded. Following a second centrifugation at 6000×g at 4° C. for 1 min, residual PEG/NaCl solution was aspirated by pipette. Phage pellets were resuspended and consolidated in minimal SM buffer (50 mM Tris-Cl pH 7.5, 100 mM NaCl, 8 mM MgSO4). Phage stocks typically achieved titers of 10¹²-10¹³ PFU/mL and were stored at 4° C.

### Phage Enumeration

T7 titers were determined using the standard double-layer agar (DLA) plaque assay using BL21 as the host strain (Liu, Y.; Huang, H.; Wang, H.; Zhang, Y., A novel approach for T7 bacteriophage genome integration of exogenous DNA. J Biol Eng 2020, 14, 2). Overnight cultures of BL21 in ΦLB were diluted 1:10 in fresh prewarmed ΦLB then incubated for 1 h at 37° C. and 250 rpm to bring cultures to log-phase. 10 μL of log-phase BL21 was mixed with 3 μL of appropriate dilutions of T7 sample, incubated for 5 min, then mixed with 1 mL of 50° C. 0.7% SM agarose (SM agarose+0.7% agarose (w/v)) and poured over 37° C. pre-warmed 60×15 mm ΦLB agar plates. The SM agarose was allowed to solidify at room temperature for 15 min, then plates were incubated at 37° C. for 1 h. Plates were then moved to a benchtop to incubate at room temperature overnight. T7 titers were calculated from plaque counts. Alternatively, titers were determined using DLA spot tests. 100 μL log-phase BL21 was mixed with 4 mL 50° C. 0.7% SM agarose then poured over 37° C. pre-warmed 100×15 mm ΦLB agar plates and allowed to solidify at room temperature for 15 min. Phage samples diluted in SM buffer were applied to solidified plates spotting with 3 μL sample. Spots were allowed to dry completely at room temp (˜10 min) then plates were incubated at 37° C. for 1 h. Finally, plates were incubated at room temperature overnight. T7 titers were calculated from the average of triplicate spots for each sample. Titers given in plaque forming units/mL (PFU/mL) were calculated from plaque counts using the following formula:

$$titer = \frac{\# plaques}{3 µL} \cdot \frac{1000 µL}{1 mL} \cdot dilution factor \qquad ( 1 )$$

### Rapid Phage Titer Estimation

A microtiter plate-based assay was used to determine approximate phage titer to inform appropriate phage sample dilutions for plaque assays using a Spectramax iD5 plate reader. E. coli BL21 carrying pJL1-sfGFP (T7 promotor-RBS-sfGFP-T7 terminator) was grown overnight at 37° C. at 250 rpm in LB+35 μg/mL kanamycin (ΦLB/kan). The overnight culture was sub-cultured 1:10 in fresh pre-warmed ΦLB+kan and allowed to incubate for a further 1 h to bring cell into log-phase. The culture was then diluted to OD600=0.3 and transferred to a Corning® black/clear-bottom 3631 non-treated microplate to 200 μL per well. The bacteria were then inoculated with 2 μL of phage samples and infection kinetics tracked via OD600 and sfGFP fluorescence (485 nm excitation/515 emission) on a Molecular Devices ID5 plate reader. In vivo T7-mediated T7 RNA polymerase expression drives sfGFP expression off of the reporter plasmid. Assay plates were incubated at 37° C. and “medium” speed orbital shaking between reads. Approximate T7 phage titers were calculated using a linear standard curve of phage known phage titers (1-10 log PFU/mL) vs time to Vₘₐₓ for relative fluorescence intensity with earlier Vₘₐₓ onset correlated with higher phage titers. Triplicate T7 standards, ΦLB, and bacteria without phage were included as controls in each assay plate. Limit-of-detection was 10 PFU/mL detectible within 2 hours of inoculation.

### Phage Genome Extraction

T7 genomes were extracted using Qiagen DNeasy Blood and Tissue Kits (Hilden, Germany) as previously described (Jakociune, D. et al., supra). Briefly, residual bacterial nucleic acids were removed from high titer lysates by 1 h incubation with nucleases at 37° C.: 450 μL lysate, 50 μL 10× nuclease buffer (100 mM Tris-Cl pH 7.5, 25 mM MgCl2, 1 mM CaCl₂)), 1 μL 1 mg/mL DNase I (Sigma-Aldrich, St. Louis, MO), and 1 μL 10 mg/mL RNase A (ThermoFisher Scientific, Roskilde, Denmark). Nucleases were inactivated by addition of 20 μL 0.5 M ethylenediaminetetraacetic acid (EDTA) pH 8.0 and incubation at 70° C. for 10 min. Then, capsids were degraded by the addition of 1.25 μL 20 mg/mL proteinase K and incubation at 56° C. for 1.5 h with gentle mixing by inversion every 30 min. Liberated T7 genomes were then purified following Qiagen DNeasy Kit Protocols. Typical yields for a 10¹² PFU/mL lysate were 100 μL of ˜500-2000 ng/L genomic DNA (gDNA). DNA integrity was confirmed by gel electrophoresis (not shown).

### Plasmid Purification and Transformation

Plasmids were purified using Qiagen kits as described by manufacturer. For cloning, plasmids were purified using Qiaprep® Spin Miniprep Kits. Plasmids used in cell-free experiments were purified using Plasmid Plus Midi Kits. CRISPRi and overexpression constructs were transformed into NEB DH5α by heat-shock. Competent cells (10 μL) were mixed with assembled vectors (1 μL) and left on ice for 30 min followed by a 30s shock at 42° C. the 5 min recovery on ice. Pre-warmed SOC broth (20 g/L tryptone, 5 g/L yeast extract, 10 mM NaCl, 2.5 mM KCl, 10 mM MgCl₂, 10 mM MgSO4 and 20 mM glucose) was added to each transformation tube which were then incubated at 37° C. at 250 rpm for 1 h then plated on appropriate selective media. BL21 was transformed by electroporation. Electrocompetent BL21 was prepared as follows. BL21 was grown overnight in LB, then sub-cultured 1:100 in 100 mL pre-warmed LB in a 500 mL Erlenmeyer flask and incubated at 37° C. at 250 rpm. When, the culture reached OD600==0.4-0.6, cells were harvested by centrifugation at 10,000×g for 5 min at 4° C. Recovered pellets were washed three times with ice-cold sterile 10% glycerol, then resuspended in 10% to achieve 50× concentration. Electrocompetent cells were aliquoted and stored at −80° C. until use. Thawed 50 μL aliquots were mixed with 50-100 ng of desired plasmid (maximum 5 μL) and kept on ice for 10 min. The cell/plasmid mixtures were transferred to chilled 1 mm gap cuvettes and exposed to a 1.8 kV pulse for 5 ms followed by immediate addition of 1 mL pre-warmed SOC. Transformants were incubated at 37° C. at 250 rpm form 1 h, then plated on selective media. Recovered transformants were plated on LB agar supplemented with appropriate antibiotics (35 μg/mL kanamycin, 35 μg/mL chloramphenicol, or 100 μg/mL ampicillin) and incubated overnight at 37° C. Plasmids were validated by Sanger sequencing by Azenta (South Plainfield, NJ). Transformants bearing sequence confirmed plasmids were banked in glycerol stocks at −80° C.

### Goldengate Cloning Protocol of CRISPRi Constructs

Gene targets were selected based only with experimentally confirmed promoter positions and transcription start sites. CRISPRi constructs were all prepared using pCRJ001 as the vector backbone by replacing a LacZα with sgRNA sequences targeting either promotors or coding sequences (CDS) as close to the start codon as possible. Where possible, stringent TTTV PAM-sites were used and less stringent TTV when the alternative was not available (Chen, P.; Zhou, J.; Wan, Y.; Liu, H.; Li, Y.; Liu, Z.; Wang, H.; Lei, J.; Zhao, K.; Zhang, Y.; Wang, Y.; Zhang, X.; Yin, L., A Cas12a ortholog with stringent PAM recognition followed by low off-target editing rates for genome editing. Genome Biol 2020, 21 (1), 78). In 50 μL reactions, pCRJ001 (1.5 μg) was digested by BsaI-HF®v2 (60 U, NEB, Ipswich, MA) @37° C. for 2 h with pipet mixing every 30 min followed by heat inactivation at 80° C. for 30 min then held at 53° C. until the ligation step. sgRNA inserts were prepared by annealing complementary 20 bp DNA oligos with 4 bp overhangs complementary sticky ends on pCRJ001 BsaI-digests. In 10 μL reactions, 10 μM of each oligo were heated to 95° C. for 5 min, then stepped down to 4° C. at 0.1° C./s. Linearized pCRJ001 and annealed inserts were then ligated at 25° C. for 2 h: 1.5 μL linear vector (45 ng, 2.25 nM), 1 μL annealed oligos (300 ng, 2000 nM), and 2.5 μL 2× Instant Sticky-end Ligase Master Mix (NEB). Ligated CRISPRi vectors were transformed into DH5α by heat-shock. Transformants were plated on LB agar+35 μg/mL chloramphenicol (cm) and 40 μg/mL 5-bromo-4-chloro-3-indolyl-β-D-galactopyranoside (X-gal) (Messing, J.; Gronenborn, B.; Müller-Hill, B.; Hofschneider, P. H., Filamentous coliphage M13 as a cloning vehicle: insertion of a HindII fragment of the lac regulatory region in M13 replicative form in vitro. Proc Natl Acad Sci USA 1977, 74 (9), 3642-3646). White colonies were selected for propagation in LB+cm and plasmids were purified. Assembly of desired constructs was confirmed by Sanger sequencing. Sequence confirmed CRISRPi vectors were then transformed into BL21 by electroporation. Validated DH5α and BL21 containing CRISPRi vectors were banked as glycerol stocks at −80° C.

Preparation of pBAD Vectors by Gibson Assembly

Overexpression vectors were prepared by Gibson assembly as previously described (Gibson, D. G.; Young, L.; Chuang, R. Y.; Venter, J. C.; Hutchison, C. A., 3rd; Smith, H. O., Enzymatic assembly of DNA molecules up to several hundred kilobases. Nat Methods 2009, 6 (5), 343-5). pBAD-sfGFP (Table B) was used as the backbone for arabinose inducible gene expression. pBAD-sfGFP was linearized by restriction digestion with XhoI and EcoRI (NEB) for 1 h @37° C. The linear vector was PCR amplified by high-fidelity NEB Q5@DNA polymerase to generate blunt-ended linear PCR products. Gene inserts were PCR amplified with Q5 using BL21 genomic DNA template liberated by boiling a colony in 100 μL nuclease-free water. PCR products were designed with 20 bp overhangs complementary to the 5′ and 3′ ends of the linearized vector to replace sfGFP while retaining regulatory elements (FIG. 12). Overexpression constructs were assembled in 5 μL reactions containing 2.5 μL NEBuilder® HiFi DNA Assembly 2× Master Mix (E2621L), 0.1 pmol gene inserts, and 0.05 pmol linear vectors incubated at 50° C. for 1 h. Assembled constructs were transformed into DH5α by heat-shock and miniprepped for sequence confirmation. Correctly assembled overexpression constructs were finally transformed into BL21 by electroporation and banked as glycerol stock at −80° C.

### Gene Expression Titration Experiments

Gene repression and overexpression assay conditions were optimized for inducer concentrations and induction timing. For CRISPRi experiments, trxA repression was evaluated by mRNA level by RT-qPCR and T7 efficiency of plating after rhamnose induction at 0.02, 0.1, 0.2, 2% (w/v) rhamnose at 2, 4, 8 and 24 h post-induction. Overexpression induction conditions were determined by overexpression of sfGFP after 4 h induction with 0.002, 0.01, 0.02, 0.1, 0.2% (w/v) arabinose. sfGFP levels were measured relative fluorescence intensity.

### RNA Extractions and RT-qPCR

RNA extractions were performed using Qiagen RNeasy Kit with enzymatic lysis and an additional DNase treatment as previously described (Hay, M.; Li, Y. M.; Ma, Y., RNA extraction of Escherichia coli grown in Lysogeny Broth for use in RT-qPCR. JEMI Methods 2017, 1, 1-6). Log-phase cells (0.5 mL, OD600=0.4-0.6) were pelleted and resuspended in 200 μL lysis buffer pH 8.5 (15 mg/mL lysozyme, 1 mg/mL proteinase K, 30 mM Tris-Cl, 1 mM EDTA) then incubated at room temperature for 10 min with gentle vortexing every 2 min. The lysate was processed using the standard Qiagen RNeasy Kit protocol including on-column DNase I treatment and eluted using 30 μL nuclease-free water. A second DNase treatment was performed using a 30 μL reaction volume containing 26 μL RNA extract, 3 μL 10× TURBOT™ DNase Buffer and 1 μL TURBO™ DNase incubated at 37° C. for 30 min. DNase activity was stopped using 3 μL DNase Inactivation reagent. RNA was quantified using a DeNovix DS-11 microvolume spectrophotometer. cDNA was generated using SuperScript™ II Reverse Transcriptase with RNA normalized to 50 ng per reaction. RT-qPCR was performed in 20 μL reaction containing 10 μL iTaq Universal SYBR® Green Supermix (2×) (Bio-Rad, Hercules, CA), 500 nM forward and reverse primers (FIG. 12), and 1 ng cDNA. Relative mRNA expression was calculated using the 2^{-ΔΔCt} method (Livak, K. J.; Schmittgen, T. D., Analysis of relative gene expression data using real-time quantitative PCR and the 2(−Delta Delta C(T)) Method. Methods 2001, 25 (4), 402-8). Expression of cysG hcaT/idnT was averaged to be used as references genes due to their stable expression in the context of induced protein expression in E. coli BL21 (Zhou, K.; Zhou, L.; Lim, Q.; Zou, R.; Stephanopoulos, G.; Too, H. P., Novel reference genes for quantifying transcriptional responses of Escherichia coli to protein overexpression by quantitative PCR. BMC Mol Biol 2011, 12, 18).

### Preparation of Gene-Knockdown or -Overexpressing Cultures for Phage Fitness Assays

E. coli BL21 carrying CRISPRi or overexpression plasmids were streaked from glycerol stocks onto ΦLB+50 μg/mL chloramphenicol (ΦLB/cm) and ΦLB+100 μg/mL ampicillin (ΦLB/amp) agar respectively to generate isolated colonies. A single colony per construct was picked to inoculate overnight selective broth cultures incubated at 37° C. at 250 rpm. These cultures were then sub-cultured 1:10 into fresh 37° C. broth and incubated at 37° C. at 250 rpm for 1 h to bring cells to log-phase. Gene repression was induced by 1% inoculation of 1.2 mL of 37° C. ΦLB/cm+2% rhamnose with log-phase cultures. Gene overexpression was induced by 1% inoculation of 1.2 mL of 37° C. ΦLB/amp+0.2% arabinose with log-phase cultures. Induced cultures were incubated in Corning Costar® 3738 Not Treated 24-well plates at 37° C. at 250 rpm for at least 4 h until OD600 reached 0.4-0.6, then placed on ice for 15 min. Induced and non-induced controls were centrifuged at 6000×g at 4° C. for 5 min, then resuspended in fresh media to OD=0.3 and kept on ice until phage fitness assays (lysis timing and efficiency of plating). Non-targeting (NT)-controls were included in each CRISPRi experiment. Overexpression of sfGFP was included as a negative control in overexpression experiments. Biological replicates (n=3) were performed on different days with cultures started from unique isolated colonies. Antibiotics and inducers were 0.22 μm filter sterilized and added to broth cultures immediately before use.

### Kinetic Assay of Lysis

Induced and non-induced control cultures were loaded into Corning Costar® 3631 black/clear-bottom 96-well plates to 200 μL and incubated at 37° C. for 15 min to pre-warm. The cultures were then inoculated with 20 μL T7 diluted in LB to a multiplicity of infection (MOI) of 3 using a multichannel pipette. ΦLB was added to phage-free controls. Lysis/growth kinetics were recorded by plate reader by measuring OD600 very 3 min with orbital shaking between reads. Lysis time was defined as the time post-infection at which a decrease in OD600 was first recorded. Mean lysis time (T_{50%}) was calculated as the midpoint of the lysis curve fit to a cumulative normal distribution (Heineman, R. H.; Bull, J. J., Testing optimality with experimental evolution: lysis time in a bacteriophage. Evolution 2007, 61 (7), 1695-709; Heineman, R. Lysis time, optimality, and the genetics of evolution in a T7 phage model system. University of Texas at Austin, 2007). Effect of gene repression or overexpression on lysis times and mean lysis times were evaluated by paired two-tailed t-test on averages from biological triplicates (p<0.05).

### Efficiency of Plating (EOP) Assay

Fifty microliters of knockdown and overexpressing BL21 strains at OD600=0.3 were infected with T7 at an MOI=0.0001 using a multichannel pipette and allowed to incubate in Greiner Bio-One #655201 polypropylene flat-bottom 96-well microplates for 30 min at 37° C. and 250 rpm. After incubation, 100 μL of ice-cold chloroform was added to each well to halt phage replication and lyse remaining intact cells. Samples were transferred to microcentrifuge tubes containing 500 μL ice-cold SM buffer and centrifuged at 17,000×g for 1 min for phase separation. The supernatants were further diluted in SM buffer as necessary. Phage titers were counted by spot tests on wild-type BL21 overlay plates from the average of three 3 μL technical replicate spots. Non-induced knockdown and overexpressing strains were included as controls. For each strain, efficiency of plating (EOP) was calculated using the following formula:

$$E O P = \frac{mean {PFU}_{induced}}{mean {PFU}_{non - induced}} \qquad ( 2 )$$

### Preparation of Cell-Extracts for CFES and CFBS

Cell-extracts were prepared based on Kwon et al. (2019) with modification. Each knockdown and overexpression strains was grown overnight in 1 mL LB, supplemented with appropriate antibiotics, inoculated by a single isolated colony. Overnight cultures were diluted 1:10 in prewarmed selective LB and incubated for 1 h at 37° C. to bring the cultures to log-phase. Next, cultures were diluted 1:100 in pre-warmed 2×YTP media (16 g/L of tryptone, 10 g/L of yeast extract, 5 g/L of sodium chloride, 7 g/L of potassium phosphate dibasic, 3 g/L of potassium phosphate monobasic, pH 7.2) plus appropriate antibiotics in 500 mL baffled-flasks and incubated at 37° C. at 250 rpm and growth monitored by OD600. When OD600 reached ˜0.2, L-rhamnose was added to knockdown cultures and L-arabinose to overexpression cultures to 2% and 0.2% respectively. When OD600 reached 2.0-3.0, flasks were immediately placed on ice and gently mixed to quickly bring down temperature. Samples were kept cold from this step onward. After 15 min on ice, each culture was centrifuge at 10,000×g for 5 min at 4° C. The supernatants were removed and pellets washed three times with ice-cold S30B buffer (10 mM Tris-Cl, pH 8.2, 14 mM magnesium glutamate, 60 mM potassium glutamate, 2 mM DTT). Pellet wet-mass (g) was recorded followed by flash-freezing in liquid nitrogen and storage at −80° C. overnight. Frozen pellets were thawed slowly in ice-water then resuspended in S30B (0.8 mL per g wet-mass) and transferred to 1.5 mL microcentrifuge tubes with final volumes 500-1000 μL. Cell slurries were lysed in an ice-water bath using the Qsonica Q125 Sonicator with ⅛″ probe set to 50% amplitude with 10 s on/off pulses to avoid overheating samples. Target total energy input was calculated using the following formula (Kim, J.; Copeland, C. E.; Seki, K.; Vogeli, B.; Kwon, Y. C., Tuning the Cell-Free Protein Synthesis System for Biomanufacturing of Monomeric Human Filaggrin. Front Bioeng Biotechnol 2020, 8, 590341):

$$Energy ( J ) = \frac{sample volume ( µL ) - 33.6}{1.8} \qquad ( 3 )$$

DTT was added to each lysate for a final 3 mM concentration. Cell-lysates were centrifuged at 12,000×g for 10 min at 4° C. Supernatants from each strain were consolidated, then incubated at 37° C. shaking for 80 min for a runoff reaction followed by another centrifugation at 10,000×g for 10 min at 4° C. Finally, these clarified supernatants were aliquoted and flash-frozen with liquid nitrogen then stored at −80° C. Total protein was measured using Piercem BCA Protein Assay Kit (ThermoFisher) using bovine serum albumin standards. Presence of residual bacteria was checked by spotting clarified lysates on LB agar and incubating overnight at 37° C. Inducers were dissolved in 2×YTP and 0.22 μm filter-sterilized. Biological duplicates were prepared for each selected knockdown and overexpression strain. Extracts based on wild-type BL21 absent plasmids were prepared as baseline references for protein and phage synthesis.

CFBS TXTL Protein Synthesis Reporter Assay (T7 gDNA+pJL1-sfGFP Cascade)

Activity of cell extracts for T7 gDNA-dependent transcription and protein synthesis was evaluated using sfGFP synthesis as a reporter. Protein synthesis was performed in 15 μL reactions incubated at 30° C. in 1.5 mL tubes with cell extracts occupying 33% volume and reaction buffer and DNA templates the remaining 67%. Reactions contained 57 mM HEPES, pH 8, 130 mM K(glu), 12 mM Mg(glu)₂, 0.4 mM NAD, 0.27 mM CoA, 0.75 mM cAMP, 2 mM spermidine, 1 mM DTT, 1.5 mM ATP and GTP, 0.9 mM CTP and UTP, 0.2 mg/mL E. coli tRNA, 0.068 mM folinic acid, 2 mM of each canonical amino acid except glutamate, 33 mM PEP, 12.66 mg/mL maltodextrin, 0.5 nM T7 gDNA, and 5 nM pJl1-sfGFP. Reaction components were mixed together and with DNA added last then kept on ice for 5 min prior to incubation at 30° C. Five microliter samples were taken at 4 h and 20 h, diluted in 20 μL 50 mM HEPES, pH 8, and sfGFP fluorescence measured as described above in Greiner Bio-One #781209 black flat-bottom 384-well plates. All reactions were run in triplicate.

### CFBS for Phage Synthesis

Bacteriophage synthesis was performed using reaction setup described above with modifications: pJL1-sfGFP was omitted and reaction buffer was supplemented with 3.5% (w/v) PEG-8000 and 0.5 mM dNTPs. At 4 h and 20 h, 3 μL samples were taken and diluted in 30 μL SM buffer. Approximate T7 titers calculated by the rapid titer estimation assay described above. More precise titers were calculated from plaque counts using the DLA method.

### Example 2: CRISPRi-Mediated trxA Knockdown Influences T7 Fitness

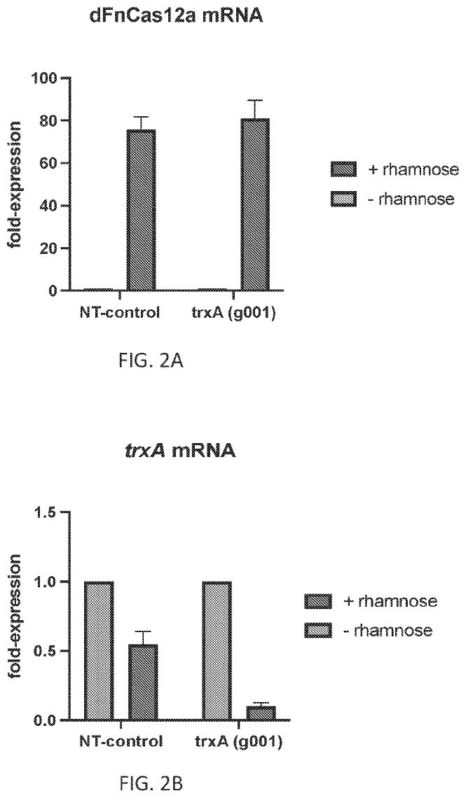

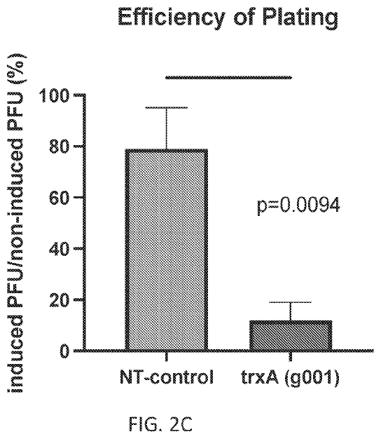

To demonstrate the efficacy of this system, a CRISPRi vector with a crRNA targeting the trxA promoter (pCRJ001_g001, inserted via Golden Gate assembly) was transformed into T7 host E. coli BL214². The trxA gene encodes thioredoxin 1, a non-essential E. coli protein involved with cytoplasmic redox homeostasis and an essential host-factor for T7 genome replication^{33, 43}. In trxA-deficient mutants, T7 will still induce host lysis and produce progeny virions, but will not replicate, making trxA an ideal target to demonstrate the effects of gene knockdown on T7 fitness⁴⁴. The functionality of the CRISPRi system was evaluated using RT-qPCR and direct plaque assays to enumerate T7 progeny resulting from infection of host E. coli BL21 with a trxA knockdown (trxA-KD) background. Gene repression was induced in log-phase cells with 2% L-rhamnose (w/v) for 4 h at which point cells were harvested for RNA extraction and T7 infection assays. RT-qPCR revealed 90±2.4% (SD, n=3) drop in trxA mRNA compared to the non-induced control carrying the same CRISPRi vector (FIG. 2B). Rhamnose induction increased dFnCas12a mRNA by over 70-fold compared to un-induced controls (FIG. 2A). T7 infection in a trxA-KD background resulted in an efficiency of plating (EOP) of 12±7% relative to the uninduced control (FIG. 2C). The trxA-KD EOP was also significantly lower compared to that of the non-targeting (NT) control strain (vector pCRJ001_g037) (p<0.01, Welch's two-tailed t-test).

### Example 3: CRISPRi Screen of Potential T7 Fitness Effector Genes

Having confirmed our inducible CRISPRi system could be used to influence T7 fitness, additional CRISPRi vectors were prepared to target each of the potential T7 effectors selected in this study (Table C).

TABLE C
CRISPRi/Overexpression gene targets.

| | | | Essential | Essential | |
|---|---|---|---|---|---|
| Target Gene | Category | Function | (E. coli)^{a} | (ΦT7)^{b} | Ref. |
| infC | protein | translation initiation factor IF-3 | Yes | — | ³³ |
| pgk | energy | phosphoglycerate kinase | Yes | — | ³³ |
| subH (ssyA) | energy | inositol monophosphatase | Yes | — | ³³ |
| hemL (gsa) | protein | glutamate-1-semialdehyde | Yes | — | ³³ |
| | | aminotransferase | | | |
| eno | energy/nucleic acid | enolase in | Yes | — | ³³ |
| | | glycolysis/gluconeogenesis/mRNA | | | |
| | | degradosome | | | |
| mukB | protein/cell-division | degradosome/chromosomal DNA | Yes | — | ³³ |
| | | condensation | | | |
| lexA | transcription factor | SOS response regulator | Yes | — | ³³ |
| rne | nucleic acid | RNase E | Yes | No | ^{30,} ^{33,} ³⁴ |
| nusG | transcription factor | transcription termination factor | Yes | No | ^{33,} ^{35,} ³⁶ |
| trx | redox regulator | oxidized thioredoxin | No | Yes | ^{30,} ³³ |
| T7gp3 | nucleic acid | ΦT7 DNA replication | No | Yes | ³⁷ |
| dgt | energy | deoxy guanosinetriphosphate (dGTP) | No | No | ^{30,} ³³ |
| | | triphosphohydrolase | | | |
| udk | nucleic acid | uridine kinase/cytidine kinase | No | No | ^{30,} ³³ |
| recC | nucleic acid | Exodeoxyribonuclease V (RecBCD) | No | No | ^{30,} ^{33,} ³⁸ |
| | | subunit | | | |
| cyaR (ryeE) | transcription factor | CyaR small RNA, oxidative stress | No | — | ³⁹ |
| | | regulator | | | |
| rna | nucleic acid | Rnase I, cleaves phosphodiester bond | No | — | ³³ |
| | | between any two nucleotides | | | |
| oxyS | transcription factor | OxyS small RNA, oxidative stress | No | No | ⁴⁰ |
| | | regulator | | | |
| trxB | redox regulator | thioredoxin reductase | No | No | ^{30,} ^{33,} ⁴¹ |

^{a}Chromosomal deletion results in non-viable or non-replicative cells.
^{b}Host/phage chromosomal deletion causes non-productive phage infections.

Two vectors were designed for each gene with one crRNA directed towards promoters and one towards coding sequences (CDS) as near to transcription start sites (TSS) as possible as available PAM sites allowed. The exception was T7 exonuclease gene 3 (T7gp3) for which only the CDS was targeted. Each CRISPRi vector was then transformed into BL21 for experiments evaluating the impact of gene knockdowns on T7 fitness.

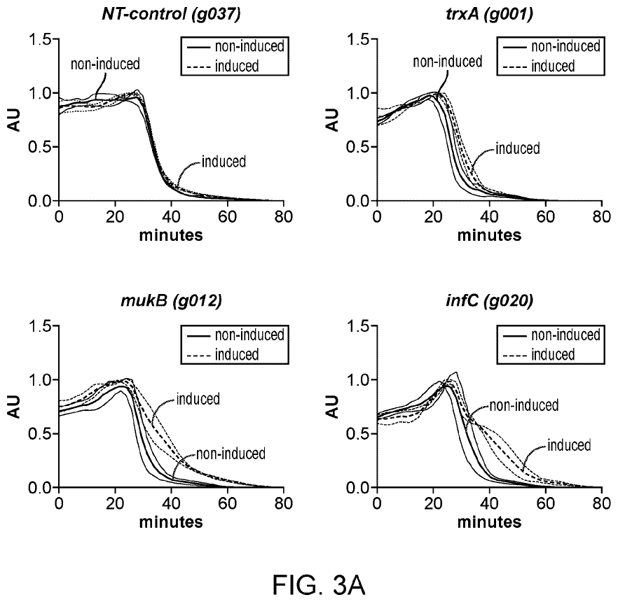

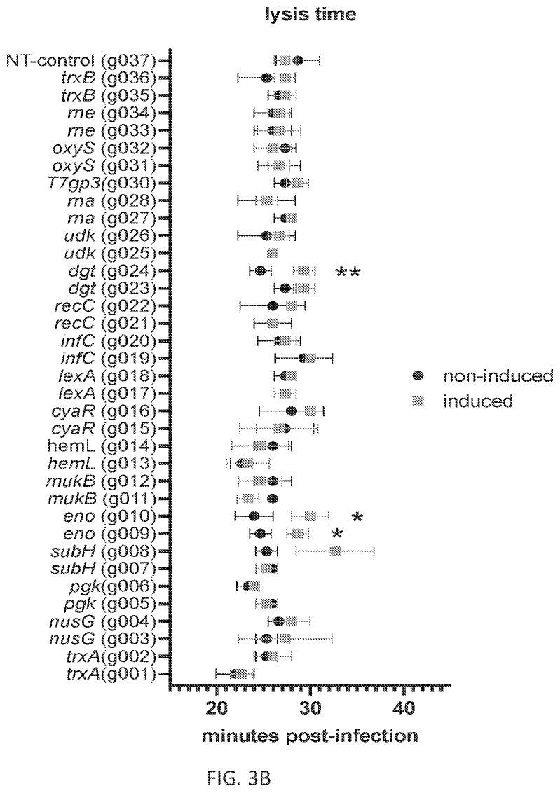

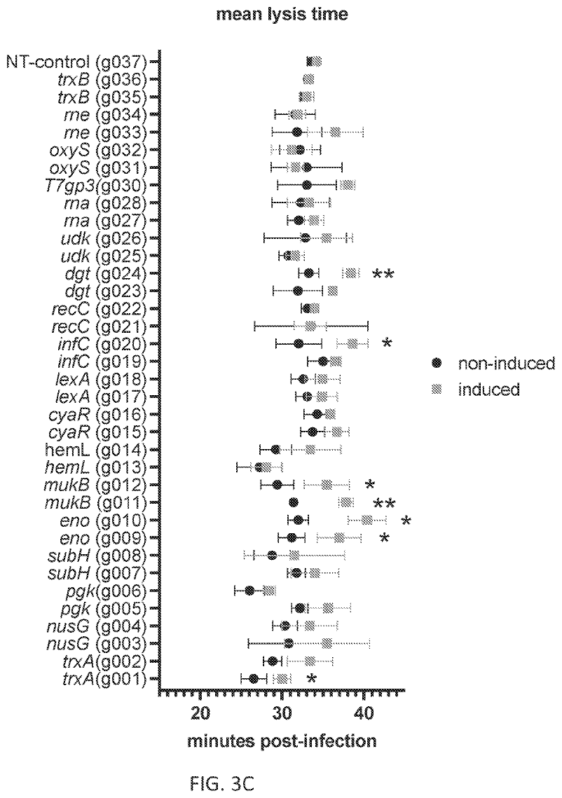

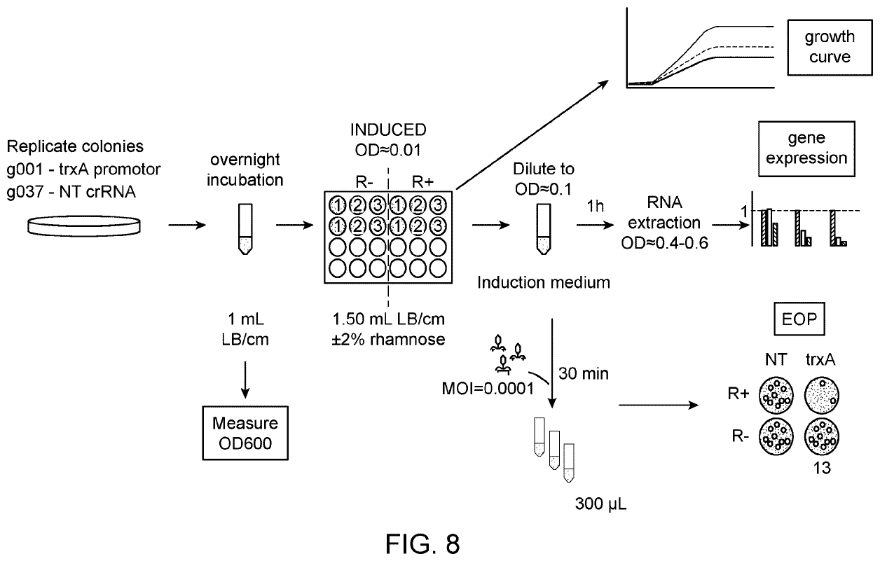

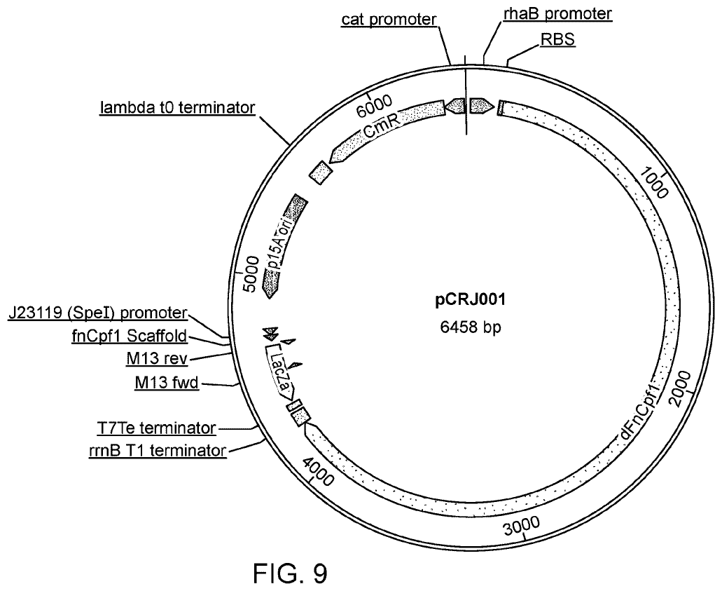

Lysis time courses and EOP assays were used to compare relative T7 fitness. Here, lysis onset time is defined as the time post-T7 infection at which optical density begins to decrease as previously described²⁶. Log-phase BL21 carrying CRISPRi vectors (OD=0.3) were infected with T7 to a multiplicity of infection (MOI)=3 then OD kinetics were tracked (FIGS. 3A and 8). Overall, in cultures without CRISPRi induction, lysis onset was ˜25 min, later than the typical T7 lysis onset of ˜15 min²⁶. We attribute this to the metabolic burden of plasmid maintenance and chloramphenicol in selective media. Initial screens found delayed lysis onset in induced CRISPRi strains with crRNAs targeting the dgt CDS (g024) (p<0.01) and both the eno promoter (g009) and CDS (g010) (p<0.05) relative to the same strains without CRISPRi induction FIG. 3B.

While only three CRISPRi targets resulted in significant changes in lysis onset time, examination of lysis curves revealed differences in the lysis kinetic profiles for many targeted genes as indicated by the widened curve profiles when CRISPRi was induced (FIG. 3A). Based on this observation, we also compared mean lysis times, which represents the time at which 50% of cells are lysed⁴⁵. In addition to dgt and eno, repressors trxA promoter (g001), mukB promotor and CDS (g011 and g012 respectively), and infC CDS (g020) were also found to have significant delays in mean lysis time. This suggests that each of these effector candidates may warrant further investigation.

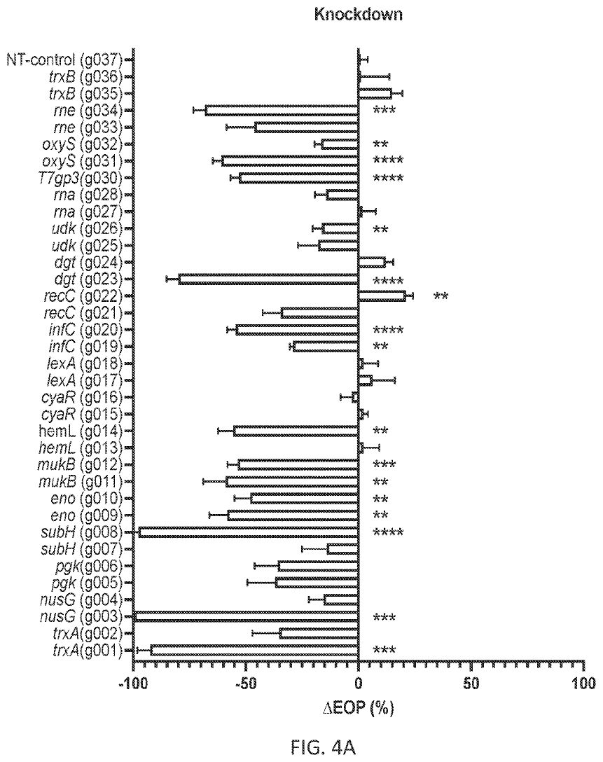

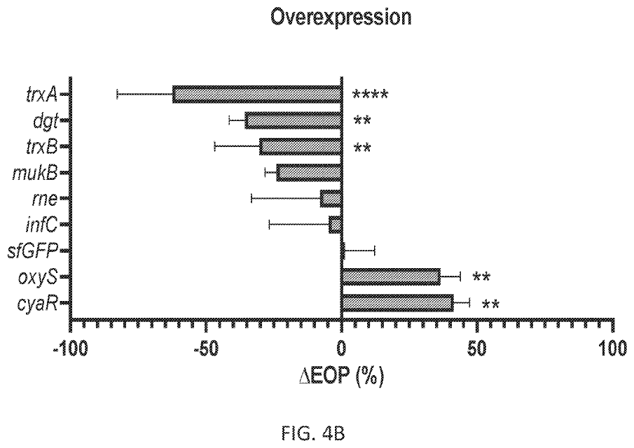

While indicative of altered T7 life cycle kinetics, lysis onset and mean lysis timing are not necessarily indicative of effects on number of progeny phage resulting from infection²⁸. Therefore, the efficiency of plating (EOP) in knockdown strains was also evaluated. Log-phase CRISPRi E. coli BL21 stains (OD=0.3) were infected at MOI=0.0001 (1 virion per 10,000 cells) for 30 min, then phage replication was halted with chloroform and progeny phage enumerated by plaque assay using wild-type BL21 as host. EOP assays found that most CRISPRi constructs had a significant negative impact on T7 titer with the exceptions of those targeting lexA, trxB, rna, pgk, cyaR, and recC (FIG. 4A). SOS response transcriptional regulator lexA expression is tightly limited under normal growth conditions, so its repression was not expected to elicit a strong effect on T7 fitness⁴⁶. Likewise, starvation response regulator cyaR repression did not affect T7 lysis nor EOP during growth on rich media⁴⁷. RNase I encoded by rna localized in the periplasm and is thus unlikely to interfere with T7 mRNA stability⁴⁸. Phosphoglycerate kinase (pgk) did not affect lysis timing or EOP, which is unexpected given the lysis delay and EOP drop induced by the enolase (eno) knockdown as both enzymes are part of the canonical glycolysis pathway⁴⁹.

eno was chosen as a CRISPRi target because of its roles in glycolysis, as part of the mRNA degradosome complex, and interactions between enolase and other phage. Notably, enolase associates with RNase E (rne), whose major role is degradation of mRNA. The T7 early gene gp0.7 inhibits RNase E activity early during infections to stabilize phage-derived mRNAs³⁴. There is evidence of protein-protein interactions between coliphage T1 and E. coli enolase (significance of interaction unknown) and direct inhibition of enolase by Bacillus subtilis phage SPO1, suggesting eno may play a role in T7 fitness directly or indirectly^{34, 50, 51} Because me inhibition is part of the T7 lifecycle, we expected me knockdown to have a neutral to positive effect on EOP. However, as with eno, me knockdown lowered EOP. This was perhaps due to impairment of RNase E's role in rRNA maturation, which could lead to reduced translational capacity. In silico modeling of infection kinetics suggests that translation rate is the primary bottleneck in T7 progeny assembly³¹. The hypothesis that translation rates influence T7 fitness is supported by our observations of lysis delay and EOP loss in translation initiation factor IF-3 (infC) (FIG. 4A).

Of the essential E. coli genes investigated, mukB, subH, hemL, and nusG repression caused significant loss of EOP (FIG. 4A). While each of these knockdown strains were viable under experimental conditions, they also experienced severe growth rate defects, which have been associated with diminished phage bursts³¹.

Knockdown of known antagonistic E. coli genes udk (uridine-cytidine kinase; uridine/cytidine+ATP→UMP/CMP+ADP+H⁺)⁵², and dgt (dGTPase; dGTP→dG+PPPi)⁵³ unexpectedly resulted in lower EOP (FIG. 4A). T7 encodes Udk and Dgt inhibitors (gp0.7 and gp1.2, respectively), so we hypothesized that repressing these genes could increase T7 fitness. However, the interfering with udk only caused a small non-significant drop in EOP. The dgt promoter repressor (dgt023) caused a significantly lower EOP, perhaps due to perturbed ribo- and deoxyribonucleotide pool homeostasis, thereby preventing normal T7 activities²⁹.

CRISPRi-based knockdown of the small regulatory RNA oxyS also lowered EOP (FIG. 4A), which was consistent with our expectation. OxyS acts as a regulator of stress-response sigma factor RpoS (σ^{S}) expression and oxidative stress response⁵⁴. Previous studies demonstrate that ΔoxyS E. coli has higher expression of RpoS and RpoS-regulated genes relative to wild-type and that inducible oxyS expression results in RpoS suppression⁵⁴. Early T7 transcription is mediated primarily by E. coli RNA polymerase-sigma 70 complex (Eσ⁷⁰), but can also be carried out using the alternate Eσ^{S} complex⁵⁵. During middle and late gene transcription, Eσ⁷⁰ and Eσ^{S} are inhibited by T7 gp2 and gp5.7, respectively, and transcription is taken over by T7 RNAP. T7 gp2 mutants experience abortive infections associated with “aberrant” transcription (interrupted transcripts and atypical, terminator read-through) caused by competition between Eσ⁷⁰ and T7 RNAP⁵⁶. We suspect that the loss of EOP in oxyS-knockdown strains may be due to incomplete Eσ^{S} inactivation due to greater background RpoS accumulation and competition between active Eσ^{S} and T7 RNAP.

Another interesting observation was the neutral impact of trxB (thioredoxin reductase) knockdown on EOP. In the TrxA/TrxB redox system, TrxA acts as a recyclable reducing agent with diverse roles in over 80 protein-protein interactions and transcriptional regulation of at least 26 genes⁵⁷. These complex interactions are implicated in most cellular processes including oxidative stress response, translation, energy transduction. TrxB recharges oxidized TrxA to restore the pool of reduced-form TrxA. Under normal conditions, balanced TrxA concentrations and TrxB activity maintains the majority of TrxA in its reduced form^{58, 59}. TrxA exists in its reduced state in the functional T7 DNA polymerase holoenzyme and does not meaningfully interact with T7 DNA polymerase in its oxidized state⁴¹. trxB deletion causes cytosolic TrxA to exist solely in its oxidized form, which has been shown to lower EOP by 10⁻⁸-fold for coliphage f1, which also requires reduced form TrxA^{41, 60} As discussed above, trxA knockdown lowered EOP, likely by depletion of free TrxA (FIGS. 2, 4A). We anticipated trxB knockdown would cause a similar effect by depleting reduced-form TrxA. The lack of impact on EOP in the trxB-KD strains suggests there is sufficient TrxB activity to supply adequate reduced-form TrxA to support T7 replication.

The recC CDS (g022) was the only target whose knockdown resulted in a significant positive effect on EOP (+21±3.1%). RecC was selected as a target because of its role its role in the double-stranded DNA repair RecBCD holoenzyme, which has exonuclease activity on linear DNA such as the T7 genome⁶¹. Deletion of recB or recC results in loss of nuclease activity³⁸. Exonuclease inhibitor GamS is typically included in CFES to protect linear DNA templates from RecBCD. Improved EOP here was consistent with recent work by Batista et al. (2022) showing that TXTL with recB or recBCD deletions improved CFES sfGFP yields for linear templates compared to wild-type TXTL¹⁵.

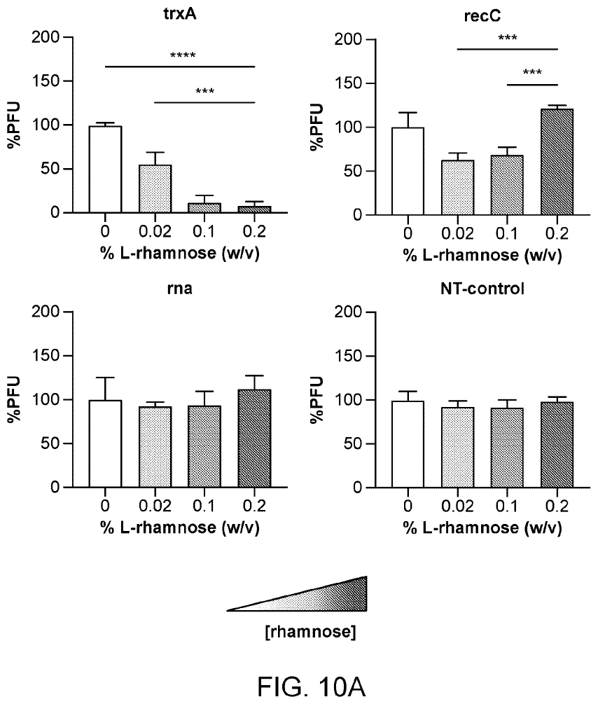

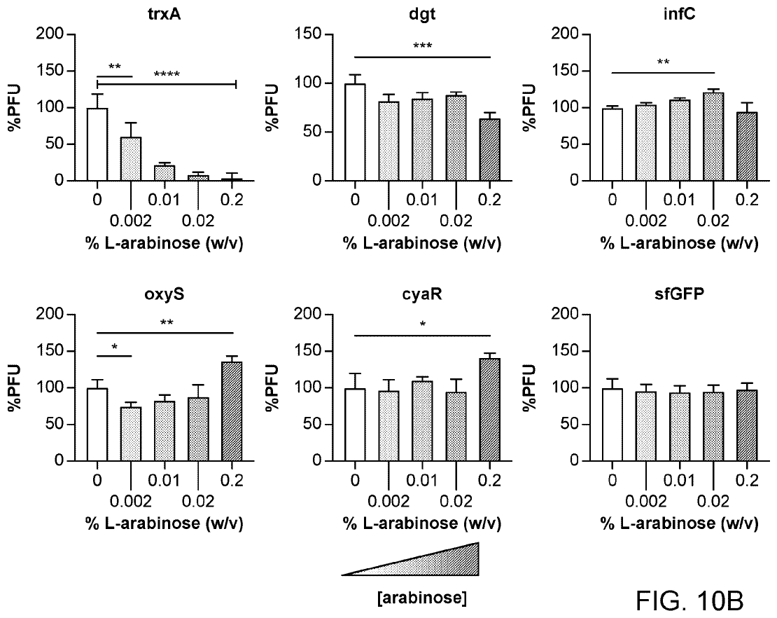

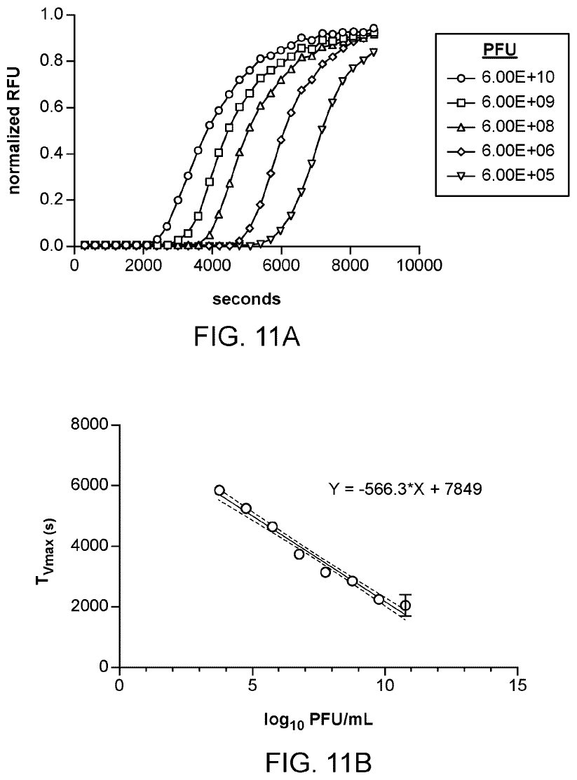

Inducer titration experiments were conducted on a subset of CRISPRi strains to determine if gene knockdown effects were tunable (FIG. 10A). The trxA knockdowns showed concentration dependence where higher L-rhamnose concentrations caused greater loss of EOP. Repression of rna had no impact on EOP at any concentration of rhamnose, consistent with our previous results (FIG. 4A). At lower rhamnose concentrations (0.02-0.1% w/v), recC repression lowered EOP in contrast to the EOP gains observed at higher concentrations (0.2-2% w/v).

In brief, we demonstrated a novel dFnCas12a-based single-plasmid CRISPRi system could be used to influence phage fitness in an inducible and tunable manner. The system can be used to knockdown essential and non-essential genes of host and phage as well as genes encoding sRNAs rather than proteins. The strength of interference with phage fitness depends on the strength of gene repression, so care must be taken when designing crRNAs as which provides tighter control, targeting promoters versus CDS, appears to be gene dependent. One approach could be to design additional crRNA constructs for each gene or to multiplex promoter and CDS targeting as our FnCas12a system is readily multiplexed by constructing vectors with consecutive guide RNA scaffold arrays⁶².

### Example 4: Overexpression of Potential Phage Fitness Effectors

To complement the investigation of phage effectors by CRISPRi, we sought to explore modulating T7 fitness by effector overexpression. L-arabinose inducible pBAD vectors were constructed to overexpress a subset of the T7 effector targets. This subset was chosen from among the genes whose repression caused significant loss in EOP (FIG. 4A) under the hypothesis that if repression causes a negative impact on T7 fitness, overexpression may have a positive impact. Among these genes, only overexpression of sRNAs oxyS and cyaR increased T7 EOP (FDR q<0.01, Welch's two-tailed t-test), dgt, trxB, and trxA decreased EOP, and infC and me had a neutral effect as did the control of overexpressed sfGFP (FIG. 4B). mukB overexpression, which has also been implicated in acetate tolerance⁶³, also decreased EOP but was not significant. In contrast to the knockdown studies, none of these overexpression impacted lysis timing nor the profile of their lysis curves (data not shown).

We suspect that CyaR and OxyS may support T7 transcription by interfering with RpoS translation^{39, 54} OxyS may also act to reduce oxidative stress on T7 by activating oxidative stress-response in an RpoS-independent manner^{64, 65}.

Huber et. al (1988) found that a Dgt overexpressing optA1 mutant E. coli maintained 50× higher dGTPase concentrations, which lowered dGTP pools 5-fold⁶⁶. Wild-type T7 is able to replicate in these dGTP depleted backgrounds, but foundational studies utilizing optA1 strains do not report impact on EOP⁶⁶⁻⁶⁸. Here, we again demonstrate that T7 does propagate in a Dgt overexpressing strain, but EOP is diminished.

Unexpectedly, TrxA and TrxB overexpression decreased EOP. We expected these genes to have a positive effect on T7 titer in contrast to the near abolition of T7 replication in trxA CRISPRi strain g001. It is unclear why this occurred, but overexpression of these genes may have interfered with redox balance or had other unpredictable effects given TrxA's many protein-protein and protein-DNA interactions^{57, 59}.

As with the CRISPRi constructs, our overexpression strains showed L-arabinose concentration dependence of EOP effects (FIG. 10B). Most notably, there is a negative relationship between L-arabinose concentration (0.002-0.2% w/v) and EOP in the pBAD-trxA strain. pBAD-oxyS and pBAD-cyaR only reach significantly increased EOP at 0.2% L-arabinose. Likewise, pBAD-dgt only significantly lower EOP at 0.2% L-arabinose. Meanwhile, pBAD-infC showed a steady but small EOP increase up to 0.02% L-arabinose, but dropped back to baseline at 0.2%. Importantly, T7 EOP was insensitive to induction of control pBAD-sfGFP at all L-arabinose concentrations.

### Example 5: Effect of Gene Expression Background in TXTL Source on Cell-Free Expression Systems and Bacteriophage Synthesis

To determine if in vivo effectors of T7 fitness had the same qualitative effect in cell-free systems, CRISPRi and overexpression strains were selected to prepare transcription/translation machinery (TXTL) for cell-free protein express systems (CFES) and cell-free bacteriophage synthesis (CFBS) based on prior research indicating their usefulness in cell-free protein synthesis or positive effects on T7 fitness during in vivo studies. Exogenous IF-3 (infC) supplementation, recC deletion, and rna deletion each have been shown to improve CFES yields of GFP^{15, 48, 69} oxyS- and cyaR-overexpression strains were chosen as positive in vivo effectors, Dgt-overexpression and trxA-knockdown strains as likely negative effectors, and trxA-overexpression for its strong negative effects. All lysates were harvested 4 h after induction with appropriate inducer and harvested in mid-log-phase with OD₆₀₀˜2-3 yielding total protein yields ranging from 15-25 mg/mL. No growth defects were observed, but recC-KD strains had an extended lag-phase (˜1 h) before entering log-phase. Lysates were produced from each strain in duplicate grown from different colonies.

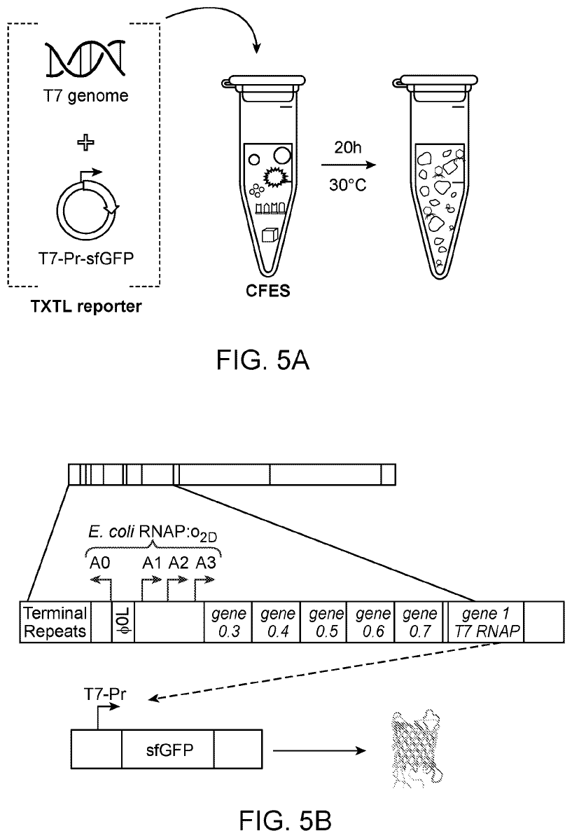

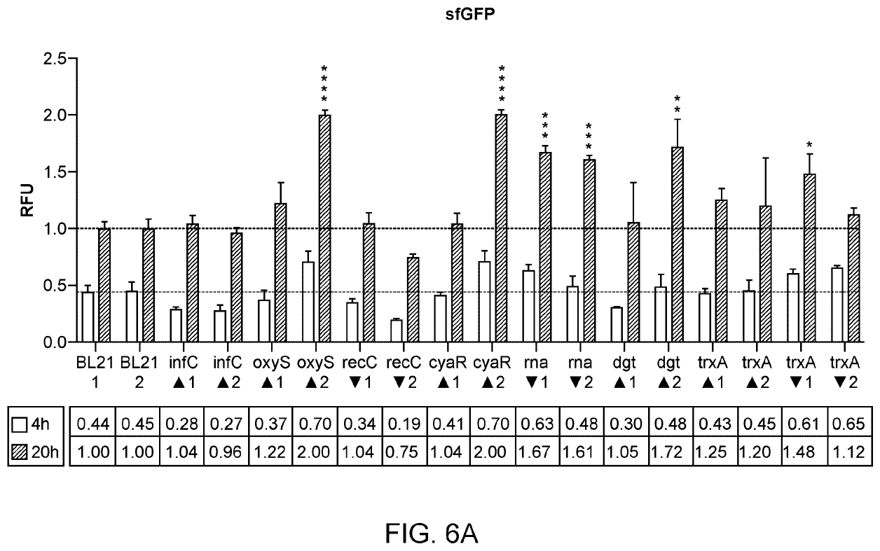

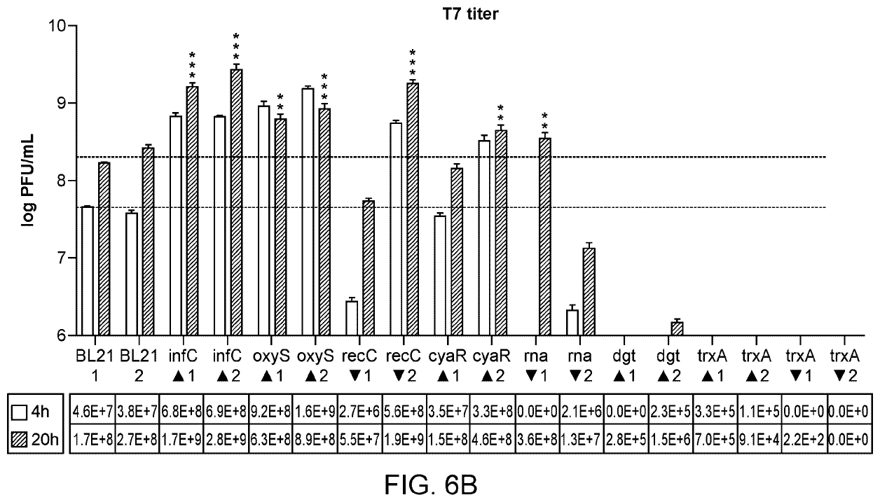

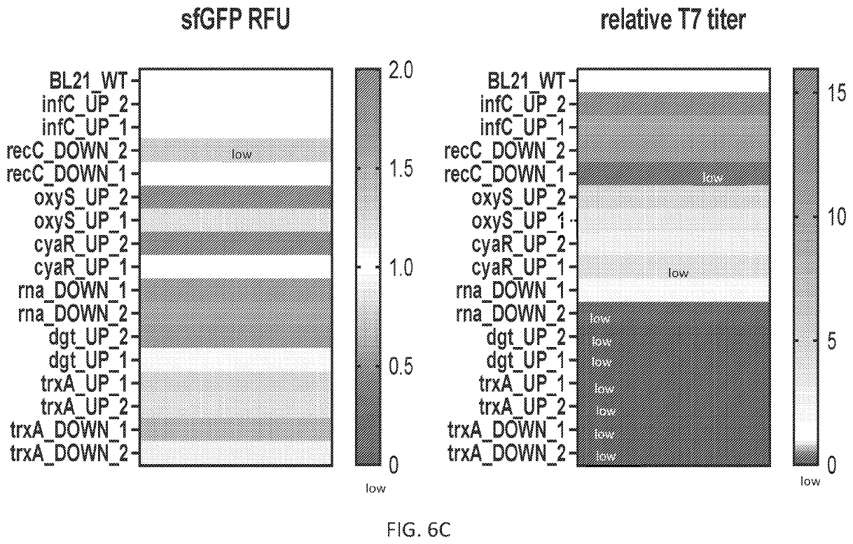

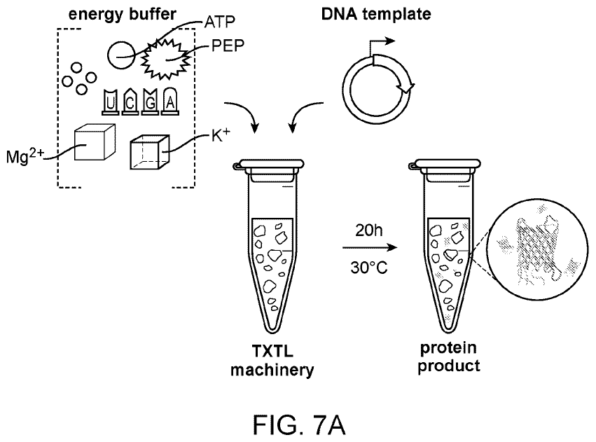

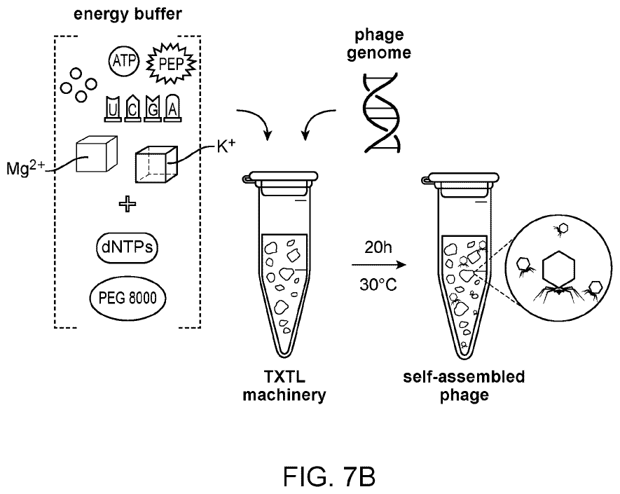

To confirm that each lysate could carry out transcription from the T7 genome and produce proteins, we developed a simple cascade CFES reaction containing T7 genomes and fluorescent reporter plasmid pJL1-T7-Pr-sfGFP (FIG. 5A). In these reactions endogenous E. coli RNA polymerase transcribes T7 early genes including T7 RNAP (gp1) (FIG. 5B). T7 RNAP then drives sfGFP expression via the reporter plasmid. sfGFP is only synthesized in the presence of functional E. coli TXTL machinery and T7 transcriptional activity. CFES using each lysate showed sfGFP yields matching (infC_UP, recC_DOWN, trxA_UP) or exceeding that of wild-type BL21 TXTL after 20 hr (oxyS UP, cyaR_UP, rna_DOWN, dgt UP) (FIG. 6A). However, these CFES reactions did not result in detectible plaque forming units (PFU).

Having confirmed all lysates were capable of T7-mediated protein synthesis, CFBS reactions were carried out. infC_UP, oxyS UP, cyaR_UP, recC_DOWN, and rna_DOWN all matched or exceeded wild-type BL21 lysate T7 yields by 20 hrs for at least one biological replicate with infC_UP lysates resulting in the greatest fold increase in T7 titer (9.7 to 16-fold) (FIG. 6B). Consistent with expectations, dgt_UP and trxA_DOWN eliminated T7 yields in some reactions and lowered yields by 10² to 10⁵-fold in reactions where T7 was detected. trxA_UP reactions also had T7 yields diminished by ˜10³-fold, which was consistent with in vivo effector assays.

It is notable that high or low CFES sfGFP yields did not necessarily predict CFBS T7 yields (FIG. 6C). For instance, infC_UP matched wild-type BL21 TXTL for sfGFP yields but increased T7 yields whereas dgt_UP and trxA_DOWN exceeded wild-type BL21 for sfGFP yields but suffered substantial loss of T7 titer. This suggests that the influence of TXTL background on phage yields is not strictly tied to protein synthesis capacity. Multiple reports describe differential optimization of cell-free synthesis of different proteins usually by varying reaction conditions (e.g. temperature, volume), composition (e.g. [Mg²⁺], linear vs. circular template, redox state), and bacterial source of transcriptional/translation machinery (e.g. E. coli A19 vs BL21 vs non-E. coli bacteria)^{15, 70-73}. These factors affect the physicochemical environment in which protein synthesis takes place and some degree of optimization may be required for each new protein or phage to be produced using cell-free systems. On the road to engineering of chassis strains for CFBS TXTL donors, further investigation is required to determine if the positive/negative effectors described here are unique to T7 or can be generalized to develop a generalized CFBS platform optimized for more production of more diverse phage.

The trends for effector impact on T7 fitness were the same in vivo and in CFBS, (e.g. positive effector in vivo and in CFBS) suggesting in vivo screening of effectors is an appropriate approach for determining TXTL backgrounds may support CFBS yield improvements.

Prior work has demonstrated that CFES supplementation with exogenous TrxA and IF-3 improved synthesis of sfGFP transcribed from plasmids by T7 RNAP⁶⁹. Here, overexpression of IF-3 increased T7 but did not have a significant impact on sfGFP yields. Conversely, TrxA overexpression negatively impacted CFBS T7 titers again without significant impact on sfGFP. These results may be explained by the impact of pleiotropic effects of overexpressed endogenous protein. IF-3 does not have any known transcriptional regulation roles whereas TrxA interacts with numerous proteins and genes with a wide variety of metabolic functions and indirectly as a transcriptional regulator⁵⁷. Likewise, OxyS and CyaR primarily function as transcription and transcription regulators of E. coli genes and may significantly alter the proteome of TXTL machinery. To avoid pleiotropic effects, future work supplementing CFBS with exogenous proteins could help elucidate whether T7 effectors influence CFES yields directly or indirectly. In particular, proteomics of oxyS- and cyaR-OX lysates may reveal more CRISPRi and OX targets to improve CFBS yields while minimizing global transcriptional changes. A combination of proteomics, CRISPRi, OX has the potential to inform the design of mutant E. coli lysates optimized for complex phage production rather than simply protein synthesis.

### Example 6: Rebooting Synthetic T7 Using CFBS Methods of the Present Technology

T7 genome fragments A, B, C, D, E, and F were generated by PCR using NEB® Q5 DNA polymerase (100 μL each) and purified phage T7 gDNA as template. PCR primers were designed to have 20 bp overlap with adjacent sequences.

PCR product size was confirmed by gel electrophoresis. After size confirmation, fragments were purified using Qiagen QiaQuick PCR purification kit.

Purified PCR products were mixed (0.2 pmol each) with NEBuilder® Hifi DNA Assembly mix from New England Biolabs (Ipswich, MA) to assemble synthetic T7 genomes. Fragments A+B+C+D generated synthetic wild-type T7 genomes. Fragments A+B+E+CASSETTE+F+D were assembled to generate synthetic T7 genomes encoding expression cassettes (“CASSETTE”) for production of heterologous proteins concomitant to T7 replication. Expression cassettes were loaded at 0.6 pmol in assembly reactions. Assembled T7 genomes were rebooted using cell-free bacteriophage synthesis: 1 μl of Gibson assembly mixture was used in 9 μL CFBS reaction mixture to reboot into functional phage.

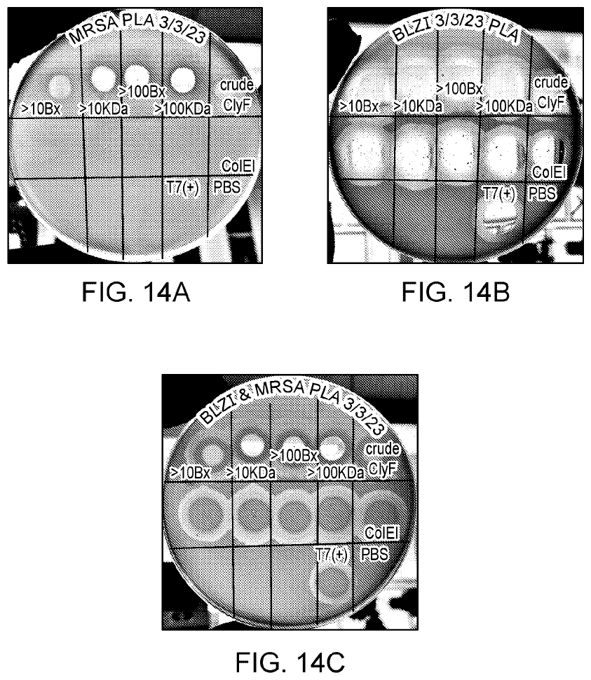

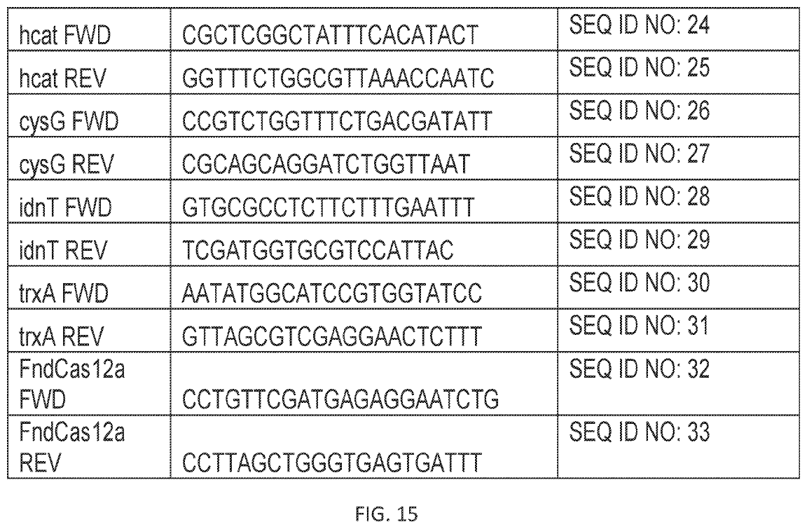

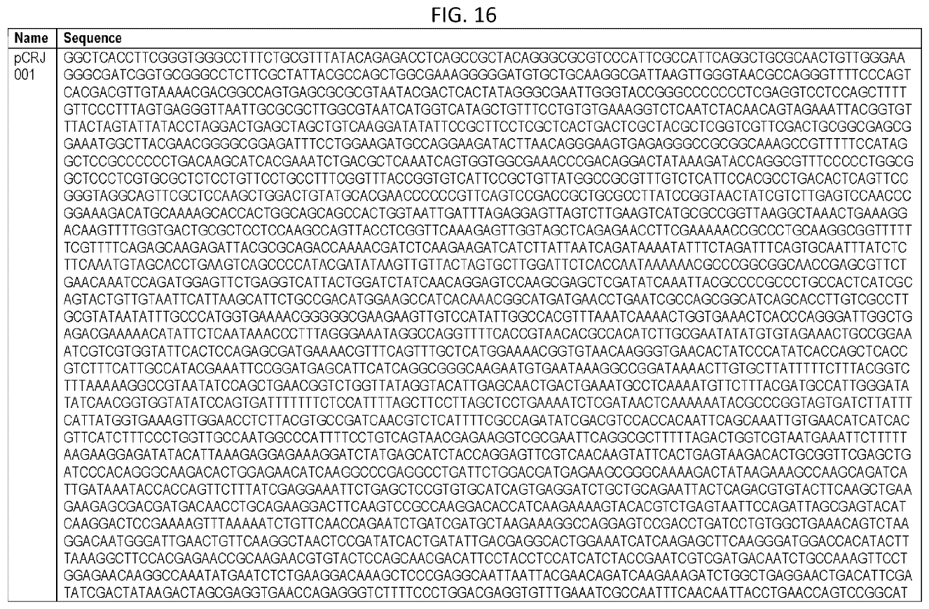

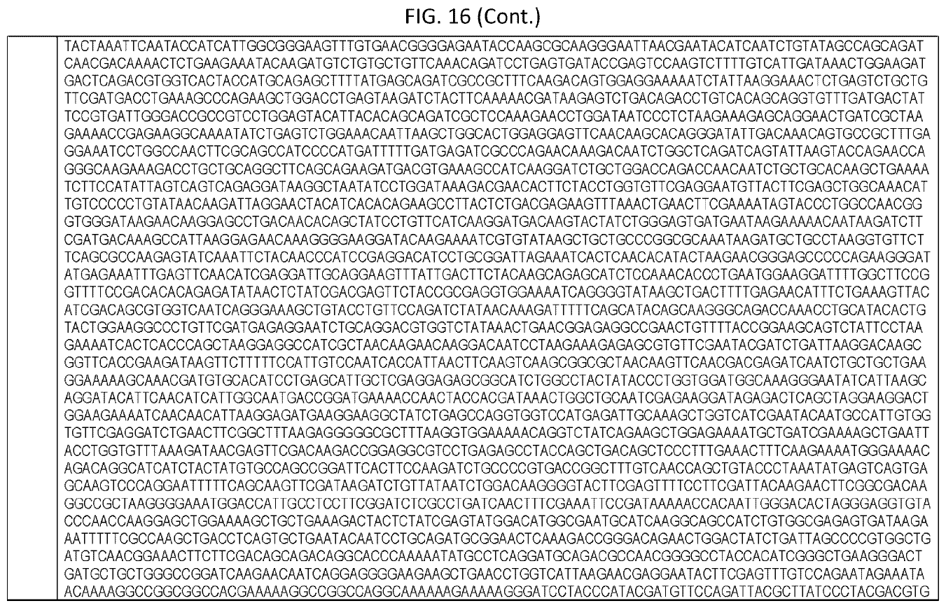

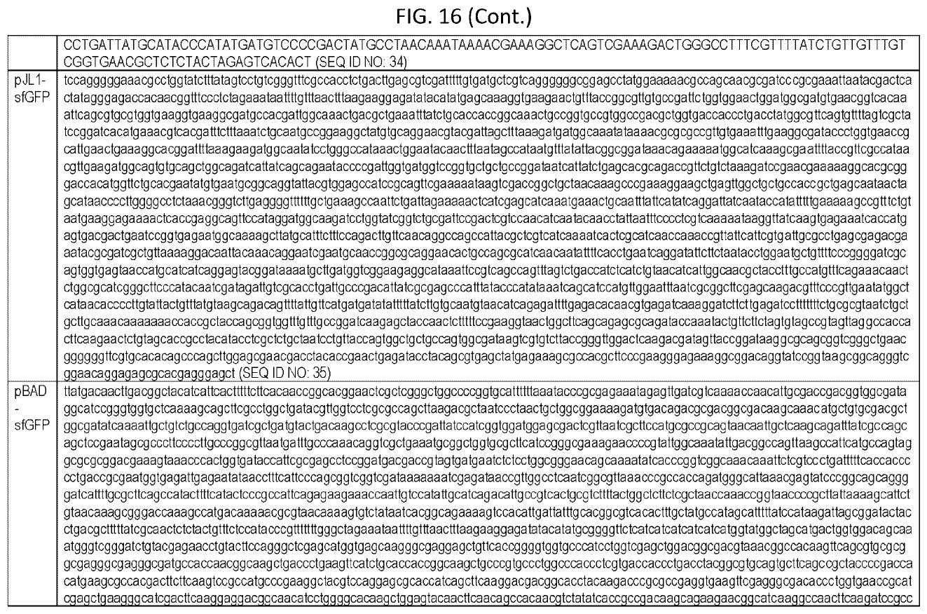

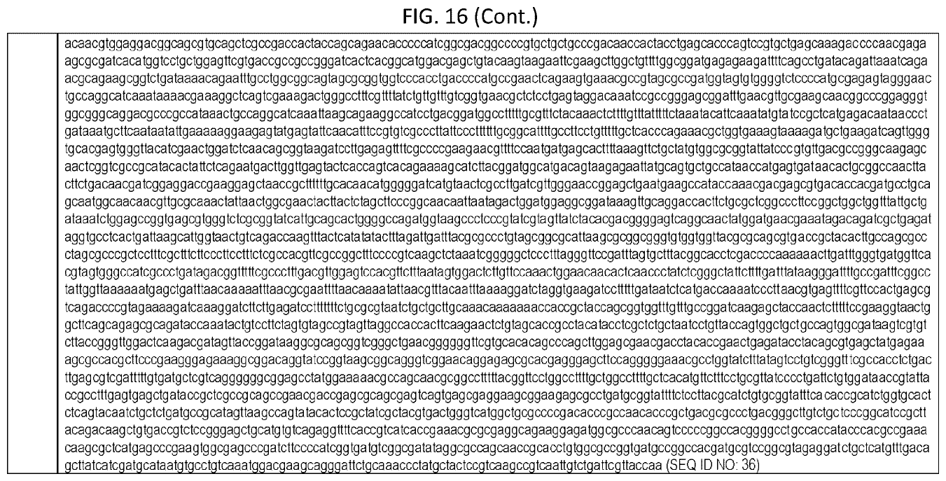

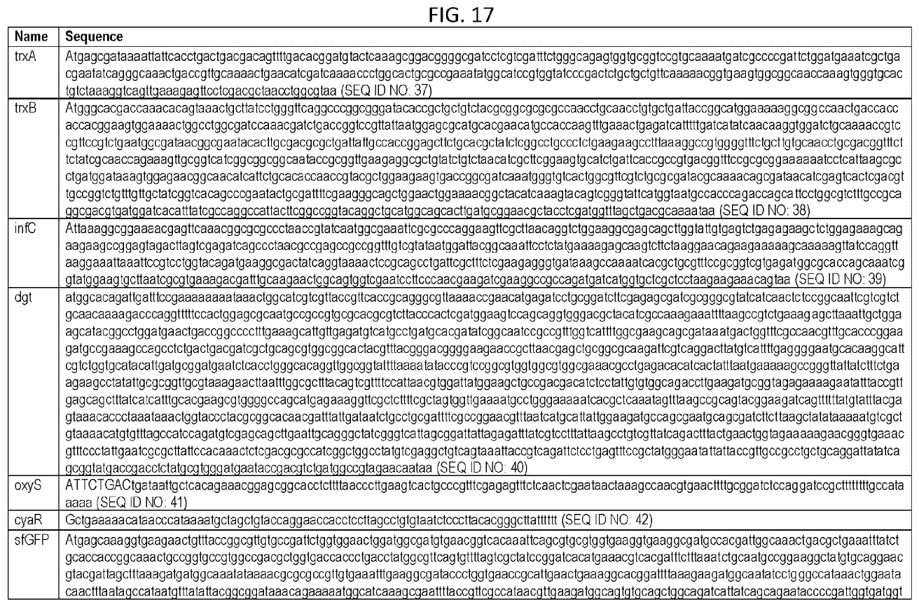

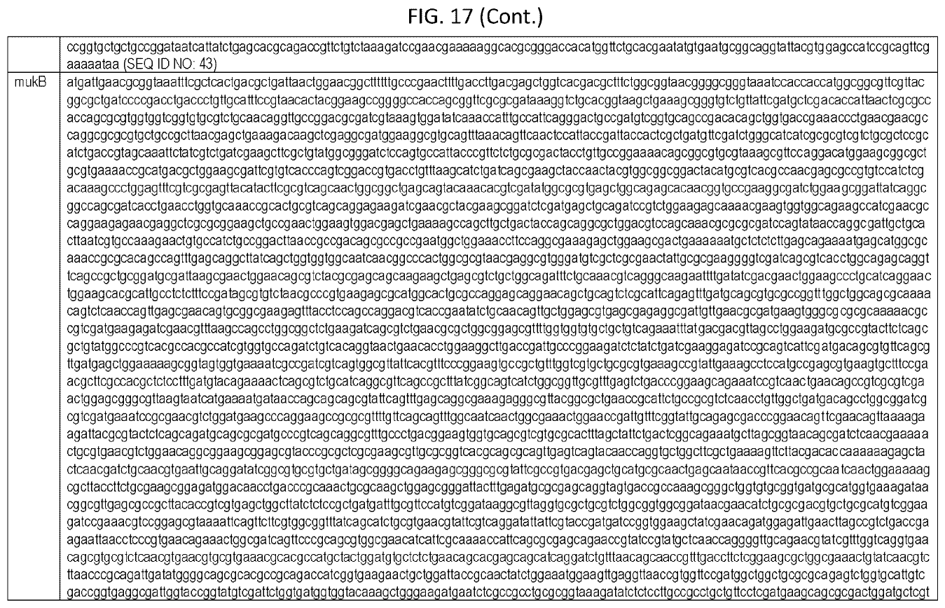

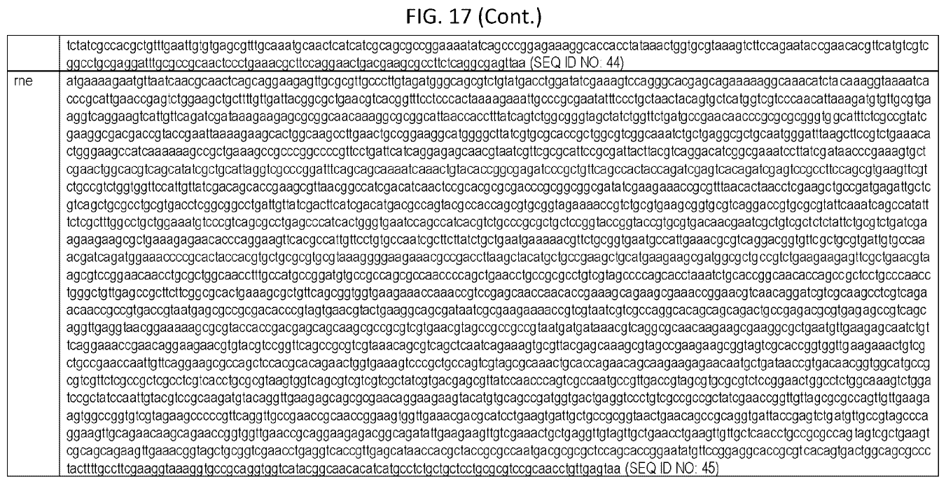

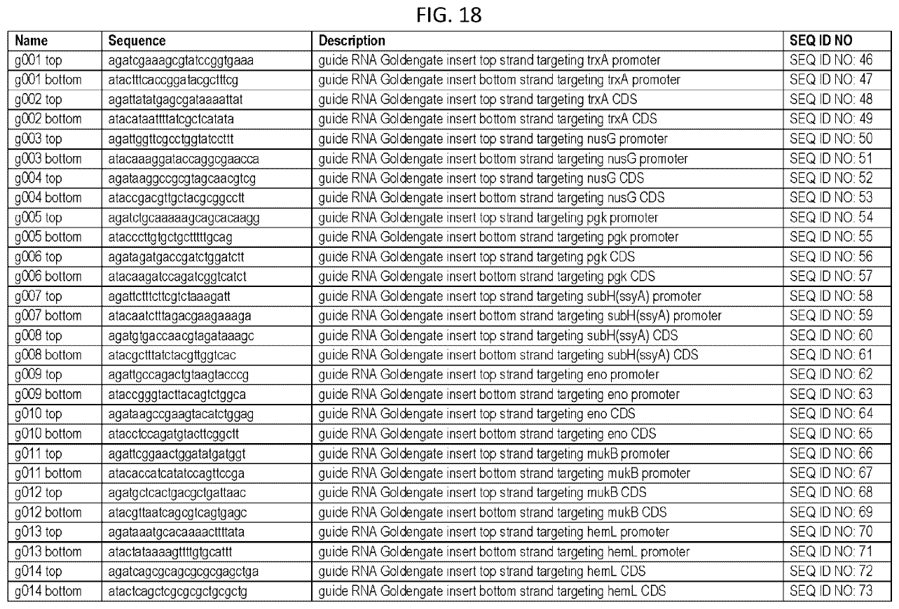

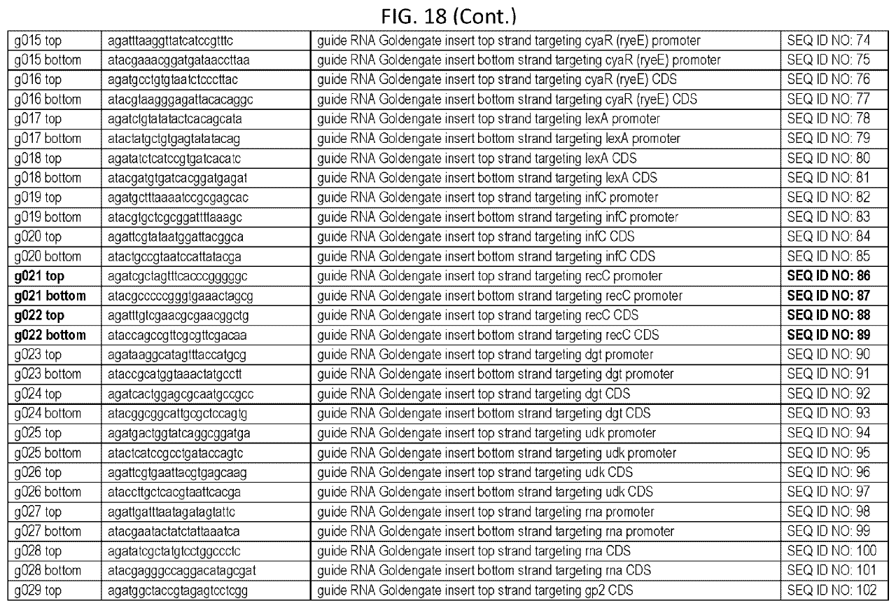

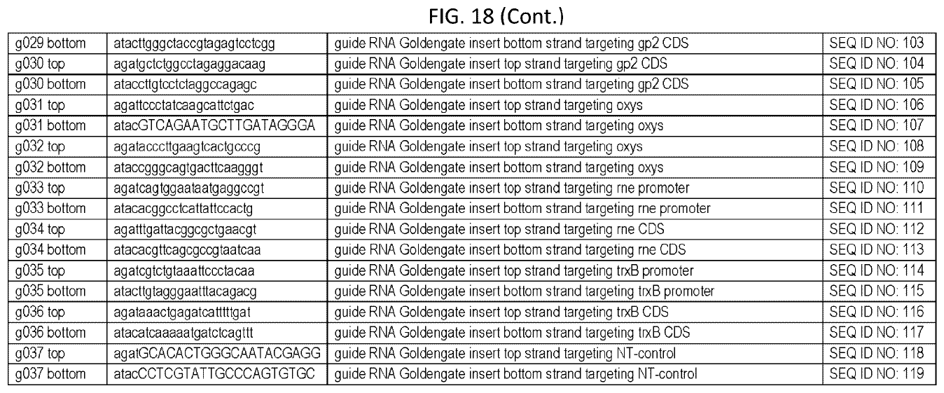

As a demonstration of this phage engineering workflow, T7_ClyF (expresses anti-MRSA antimicrobial peptide) and T7_sfGFP were assembled and rebooted entirely in vitro. See FIGS. 13-14.

Without wishing to be bound by theory, it is believed that these improved CFBS yields achieved with the methods of the present technology can be replicated in T7-like viruses given their structural and behavioral similarities to T7 phage. See e.g., Volozhantsev et al., Arch Virol (2016) 161:499-501; Lavigne et al., Virology 312 (2003) 49-59 and Leon-Velarde et al., Virology Journal 2014, 11:188.

### Example 7: CFBS Methods of the Present Technology with Different Phage Types

The CFBS methods described in Example 1 will be applied to additional phage types such as one or more of Lambda, K1E, T3, T5, T4, and PhiX174. It is anticipated that lysates obtained from genetically modified bacterial host cells (e.g., E. coli) that overexpress one or more of translation initiation factor IF-3 (infC), OxyS and CyaR and/or repress RecC subunit exonuclease RecBCD will achieve enhanced CFBS yields with one or more of these additional phage types relative to conventional CFBS methods.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof Δny listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. ANTIBIOTIC RESISTANCE THREATS IN THE UNITED STATES. Antibiotic Resistance Coordination and Strategy Unit within the Division of Healthcare Quality Promotion, National Center for Emerging and Zoonotic Infectious Diseases, Centers for Disease Control and Prevention: CDC, 2019.
2. Antibiotic Resistance Threats in the United States; US Centers for Disease Control: 2019.
3. Hyman, P., Phages for Phage Therapy: Isolation, Characterization, and Host Range Breadth. Pharmaceuticals (Basel) 2019, 12 (1).
4. Yehl, K.; Lemire, S.; Yang, A. C.; Ando, H.; Mimee, M.; Torres, M. T.; de la Fuente-Nunez, C.; Lu, T. K., Engineering Phage Host-Range and Suppressing Bacterial Resistance through Phage Tail Fiber Mutagenesis. Cell 2019, 179 (2), 459-469 e9.
5. Panwar, R. B.; Sequeira, R. P.; Clarke, T. B., Microbiota-mediated protection against antibiotic-resistant pathogens. Genes Immun 2021, 22 (5-6), 255-267.
6. Glonti, T.; Pirnay, J. P., In Vitro Techniques and Measurements of Phage Characteristics That Are Important for Phage Therapy Success. Viruses 2022, 14 (7).
7. Wilding, K. M.; Porter Hunt, J.; Wilkerson, J. W.; Funk, P. J.; Swensen, R. L.; Christian, M. L.; Bundy, B. C., Endotoxin-free E. coli-based cell-free protein synthesis: Pre-expression endotoxin removal approaches for on-demand cancer therapeutic production. Biotechnology Journal 2019, 14 (3).
8. Van Belleghem, J. D.; Merabishvili, M.; Vergauwen, B.; Lavigne, R.; Vaneechoutte, M., A comparative study of different strategies for removal of endotoxins from bacteriophage preparations. J Microbiol Methods 2017, 132, 153-159.
9. Lynch, S. Phage down under: stability of a travelling phage Capsid & Tail [Online], 2022. https://phage.directory/capsid/phage-shipping-around-australia.
10. Duyvejonck, H.; Merabishvili, M.; Vaneechoutte, M.; de Soir, S.; Wright, R.; Friman, V. P.; Verbeken, G.; De Vos, D.; Pirnay, J. P.; Van Mechelen, E.; Vermeulen, S. J. T., Evaluation of the Stability of Bacteriophages in Different Solutions Suitable for the Production of Magistral Preparations in Belgium. Viruses 2021, 13 (5).
11. Jonczyk, E.; Klak, M.; Miedzybrodzki, R.; Gorski, A., The influence of external factors on bacteriophages—review. Folia Microbiol (Praha) 2011, 56 (3), 191-200.
12. Silverman, A. D.; Karim, A. S.; Jewett, M. C., Cell-free gene expression: an expanded repertoire of applications. Nat Rev Genet 2020, 21 (3), 151-170.
13. Caschera, F.; Noireaux, V., Synthesis of 2.3 mg/ml of protein with an all Escherichia coli cell-free transcription-translation system. Biochimie 2014, 99, 162-8.
14. Romantseva, E.; Alperovich, N.; Ross, D.; Lund, S. P.; Strychalski, E. A., Effects of DNA template preparation on variability in cell-free protein production. Synth Biol (Oxf) 2022, 7 (1), ysac015.
15. Batista, A. C.; Levrier, A.; Soudier, P.; Voyvodic, P. L.; Achmedov, T.; Reif-Trauttmansdorff, T.; DeVisch, A.; Cohen-Gonsaud, M.; Faulon, J. L.; Beisel, C. L.; Bonnet, J.; Kushwaha, M., Differentially Optimized Cell-Free Buffer Enables Robust Expression from Unprotected Linear DNA in Exonuclease-Deficient Extracts. ACS Synth Biol 2022, 11 (2), 732-746.
16. Jew, K.; Smith, P. E. J.; So, B.; Kasman, J.; Oza, J. P.; Black, M. W., Characterizing and Improving pET Vectors for Cell-free Expression. Front Bioeng Biotechnol 2022, 10, 895069.
17. Guzman-Chavez, F.; Arce, A.; Adhikari, A.; Vadhin, S.; Pedroza-Garcia, J. A.; Gandini, C.; Ajioka, J. W.; Molloy, J.; Sanchez-Nieto, S.; Varner, J. D.; Federici, F.; Haseloff, J., Constructing Cell-Free Expression Systems for Low-Cost Access. ACS Synth Biol 2022, 11 (3), 1114-1128.
18. Silverman, A. D.; Kelley-Loughnane, N.; Lucks, J. B.; Jewett, M. C., Deconstructing Cell-Free Extract Preparation for in Vitro Activation of Transcriptional Genetic Circuitry. ACS Synth Biol 2019, 8 (2), 403-414.
19. Garenne, D.; Thompson, S.; Brisson, A.; Khakimzhan, A.; Noireaux, V., The all-E. coli TXTL toolbox 3.0: new capabilities of a cell-free synthetic biology platform. Synth Biol (Oxf) 2021, 6 (1), ysabo17.
20. Rustad, M.; Eastlund, A.; Jardine, P.; Noireaux, V., Cell-free TXTL synthesis of infectious bacteriophage T4 in a single test tube reaction. Synth Biol (Oxf) 2018, 3 (1), ysy002.
21. Sun, Z. Z.; Hayes, C. A.; Shin, J.; Caschera, F.; Murray, R. M.; Noireaux, V., Protocols for implementing an Escherichia coli based TX-TL cell-free expression system for synthetic biology. J Vis Exp 2013, (79), e50762.
22. Wilding, K. M.; Zhao, E. L.; Earl, C. C.; Bundy, B. C., Thermostable lyoprotectant-enhanced cell-free protein synthesis for on-demand endotoxin-free therapeutic production. N Biotechnol 2019, 53, 73-80.
23. Hunt, J. P.; Yang, S. O.; Wilding, K. M.; Bundy, B. C., The growing impact of lyophilized cell-free protein expression systems. Bioengineered 2017, 8 (4), 325-330.
24. Falgenhauer, E.; von Schonberg, S.; Meng, C.; Muckl, A.; Vogele, K.; Emslander, Q.; Ludwig, C.; Simmel, F. C., Evaluation of an E. coli Cell Extract Prepared by Lysozyme-Assisted Sonication via Gene Expression, Phage Assembly and Proteomics. Chembiochem 2021, 22 (18), 2805-2813.
25. Garenne, D.; Bowden, S.; Noireaux, V., Cell-free expression and synthesis of viruses and bacteriophages: applications to medicine and nanotechnology. Curr Opin Syst Biol 2021, 28.
26. Xu, H.; Bao, X.; Hong, W.; Wang, A.; Wang, K.; Dong, H.; Hou, J.; Govinden, R.; Deng, B.; Chenia, H. Y., Biological Characterization and Evolution of Bacteriophage T7-big up tri, openholin During the Serial Passage Process. Front Microbiol 2021, 12, 705310.
27. Shin, J.; Jardine, P.; Noireaux, V., Genome replication, synthesis, and assembly of the bacteriophage T7 in a single cell-free reaction. ACS Synth Biol 2012, 1 (9), 408-13.
28. Heineman, R. H.; Bull, J. J., Testing optimality with experimental evolution: lysis time in a bacteriophage. Evolution 2007, 61 (7), 1695-709.
29. Mutalik, V. K.; Adler, B. A.; Rishi, H. S.; Piya, D.; Zhong, C.; Koskella, B.; Kutter, E. M.; Calendar, R.; Novichkov, P. S.; Price, M. N.; Deutschbauer, A. M.; Arkin, A. P., High-throughput mapping of the phage resistance landscape in E. coli. PLoS Biol 2020, 18 (10), e3000877.
30. Qimron, U.; Marintcheva, B.; Tabor, S.; Richardson, C. C., Genomewide screens for Escherichia coli genes affecting growth of T7 bacteriophage. Proc Natl Acad Sci USA 2006, 103 (50), 19039-44.
31. You, L.; Suthers, P. F.; Yin, J., Effects of Escherichia coli physiology on growth of phage T7 in vivo and in silico. J Bacteriol 2002, 184 (7), 1888-94.
32. Joseph, R. C.; Sandoval, N. R., Single and multiplexed gene repression in solventogenic Clostridium via Cas12a-based CRISPR interference. Synth Syst Biotechnol 2023, 8 (1), 148-156.
33. Keseler, I. M.; Gama-Castro, S.; Mackie, A.; Billington, R.; Bonavides-Martinez, C.; Caspi, R.; Kothari, A.; Krummenacker, M.; Midford, P. E.; Muniz-Rascado, L.; Ong, W. K.; Paley, S.; Santos-Zavaleta, A.; Subhraveti, P.; Tierrafria, V. H.; Wolfe, A. J.; Collado-Vides, J.; Paulsen, I. T.; Karp, P. D., The EcoCyc Database in 2021. Front Microbiol 2021, 12, 711077.
34. Marchand, I.; Nicholson, A. W.; Dreyfus, M., Bacteriophage T7 protein kinase phosphorylates RNase E and stabilizes mRNAs synthesized by T7 RNA polymerase. Mol Microbiol 2001, 42 (3), 767-776.
35. Emily C. A. Goodall, a. A. R., a lain G. Johnston, a Sara Jabbari, a Keith A. Turner, b Adam F. Cunningham, a; Peter A. Lund, a. J. A. C., a Ian R. Hendersona, The Essential Genome of Escherichia coli K-12. mBIo 2018, 9 (1).
36. Burova, E.; Hung, S. C.; Sagitov, V.; Stitt, B. L.; Gottesman, M. E., Escherichia coli NusG Protein Stimulates Transcription Elongation Rates In Vivo and In Vitro. J Bacteriol 1995, 177 (5), 1388-1392.
37. Massy, B. d.; A. Weisberg, R.; Studier, F. W., Gene 3 endonuclease of bacteriophage T7 resolves conformationally branched structures in double-stranded DNA. J Mol Biol 1987, 193, 359-376.
38. Seroka, K.; Wackernagel, W., In vivo effects of recBC DNase, exonuclease I, and DNA polymerases of Escherichia coli on the infectivity of native and single-stranded DNA of bacteriophage T7. J Virol 1977, 21 (3).
39. Kim, W.; Lee, Y., Mechanism for coordinate regulation of rpoS by sRNA-sRNA interaction in Escherichia coli. RNA Biol 2020, 17 (2), 176-187.
40. Barshishat, S.; Elgrably-Weiss, M.; Edelstein, J.; Georg, J.; Govindarajan, S.; Haviv, M.; Wright, P. R.; Hess, W. R.; Altuvia, S., OxyS small RNA induces cell cycle arrest to allow DNA damage repair. EMBO J 2018, 37 (3), 413-426.
41. Russel, M.; Model, P., Direct cloning of the trxB gene that encodes thioredoxin reductase. J Bacteriol 1985, 163 (1), 238-242.
42. Chitboonthavisuk, C.; Luo, C. H.; Huss, P.; Fernholz, M.; Raman, S., Engineering a Dynamic Controllable Infectivity Switch in Bacteriophage T7. ACS Synth Biol 2022, 11 (1), 286-296.
43. Adler, S.; Modrich, P., T7-induced DNA polymerase. Requirement for thioredoxin sulfhydryl groups. Journal of Biological Chemistry 1983, 258 (11), 6956-6962.
44. Himawan, J. S.; Richardson, C. C., Genetic analysis of the interaction between bacteriophage T7 DNA polymerase and Escherichia coli thioredoxin. Proc Natl Acad Sci USA 1992, 89 (20), 9774-9778.
45. Heineman, R. Lysis time, optimality, and the genetics of evolution in a T7 phage model system. University of Texas at Austin, 2007.
46. Yaguchi, K.; Mikami, T.; Igari, K.; Yoshida, Y.; Yokoyama, K.; Makino, K., Identification of LexA regulated promoters in Escherichia coli O157:H7. J Gen Appl Microbiol 2011, 57, 219-230.
47. De Lay, N.; Gottesman, S., The Crp-activated small noncoding regulatory RNA CyaR (RyeE) links nutritional status to group behavior. J Bacteriol 2009, 191 (2), 461-76.
48. Snapyan, M.; Robin, S.; Yeretssian, G.; Lecocq, M.; Marc, F.; Sakanyan, V., Cell-Free Protein Synthesis by Diversifying Bacterial Transcription Machinery. Bio Tech 2021, 10 (4).
49. Morita, T.; Kawamoto, H.; Mizota, T.; Inada, T.; Aiba, H., Enolase in the RNA degradosome plays a crucial role in the rapid decay of glucose transporter mRNA in the response to phosphosugar stress in Escherichia coli. Mol Microbiol 2004, 54 (4), 1063-75.
50. Gassner, C.; Schneider-Scherzer, E.; Lottspeich, F.; Schweiger, M.; Auer, B., Escherichia coli bacteriophage T1 DNA methyltransferase appears to interact with Escherichia coli enolase. Biological Chemistry 1998, 379 (4-5), 621-623.
51. Zhang, K.; Li, S.; Wang, Y.; Wang, Z.; Mulvenna, N.; Yang, H.; Zhang, P.; Chen, H.; Li, Y.; Wang, H.; Gao, Y.; Wigneshweraraj, S.; Matthews, S.; Zhang, K.; Liu, B., Bacteriophage protein PEIP is a potent Bacillus subtilis enolase inhibitor. Cell Rep 2022, 40 (1), 111026.
52. Gawel, D.; Hamilton, M. D.; Schaaper, R. M., A novel mutator of Escherichia coli carrying a defect in the dgt gene, encoding a dGTP triphosphohydrolase. J Bacteriol 2008, 190 (21), 6931-9.
53. Shadrin, A.; Sheppard, C.; Savalia, D.; Severinov, K.; Wigneshweraraj, S., Overexpression of Escherichia coli udk mimics the absence of T7 Gp2 function and thereby abrogates successful infection by T7 phage. Microbiology (Reading) 2013, 159 (Pt 2), 269-274.
54. Zhang, A.; Altuvia, S.; Tiwari, A.; Argaman, L.; Hengge-Aronis, R.; Storz, G., The OxyS regulatory RNA represses rpoS translation and binds the Hfq (HF-I) protein. EMBO J 1998, 17 (20), 6061-6068.
55. Tabib-Salazar, A.; Liu, B.; Barker, D.; Burchell, L.; Qimron, U.; Matthews, S. J.; Wigneshweraraj, S., T7 phage factor required for managing RpoS in Escherichia coli. Proc Natl Acad Sci USA 2018, 115 (23), E5353-E5362.
56. Savalia, D.; Robins, W.; Nechaev, S.; Molineux, I.; Severinov, K., The role of the T7 Gp2 inhibitor of host RNA polymerase in phage development. J Mol Biol 2010, 402 (1), 118-26.
57. Kumar, J. K.; Tabor, S.; Richardson, C. C., Proteomic analysis of thioredoxin-targeted proteins in Escherichia coli. Proc Natl Acad Sci USA 2004, 101 (11), 3759-64.
58. Jurado, P.; de Lorenzo, V.; Fernandez, L. A., Thioredoxin fusions increase folding of single chain Fv antibodies in the cytoplasm of Escherichia coli: evidence that chaperone activity is the prime effect of thioredoxin. J Mol Biol 2006, 357 (1), 49-61.
59. Miranda-Vizuete, A.; Rodriguez-Ariza, A.; Toribio, F.; Holmgren, A.; Lopez-Barea, J.; Pueyo, C., The levels of ribonucleotide reductase, thioredoxin, glutaredoxin 1, and GSH are balanced in Escherichia coli K12. J Biol Chem 1996, 271 (32), 19099-103.
60. Russel, M.; Model, P., Thioredoxin is required for filamentous phage assembly. Proc Natl Acad Sci USA 1985, 82.
61. Dillingham, M. S.; Kowalczykowski, S. C., RecBCD Enzyme and the Repair of Double-Stranded DNA Breaks. Microbiology and Molecular Biology Reviews 2008, 72 (4), 642-671.
62. Liao, C.; Ttofali, F.; Slotkowski, R. A.; Denny, S. R.; Cecil, T. D.; Leenay, R. T.; Keung, A. J.; Beisel, C. L., Modular one-pot assembly of CRISPR arrays enables library generation and reveals factors influencing crRNA biogenesis. Nat Commun 2019, 10 (1), 2948.
63. Sandoval, N. R.; Mills, T. Y.; Zhang, M.; Gill, R. T., Elucidating acetate tolerance in E. coli using a genome-wide approach. Metab Eng 2011, 13 (2), 214-24.
64. González-Flecha, B.; Demple, B., Role for the oxyS Gene in Regulation of Intracellular Hydrogen Peroxide in Escherichia coli. J Bacteriol 1999, 181 (12), 3833-3836.
65. Loison, P.; Majou, D.; Gelhaye, E.; Boudaud, N.; Gantzer, C., Impact of reducing and oxidizing agents on the infectivity of Qbeta phage and the overall structure of its capsid. FEMS Microbiol Ecol 2016, 92 (11).
66. Myers, J. A.; Beauchamp, B. B.; Richardson, C. C., Gene 1.2 protein of bacteriophage T7. Effect on deoxyribonucleotide pools. Journal of Biological Chemistry 1987, 262 (11), 5288-5292.
67. Huber, H. E.; Beauchamp, B. B.; Richardson, C. C., Escherichia coli dGTP triphosphohydrolase is inhibited by gene 1.2 protein of bacteriophage T7. Journal of Biological Chemistry 1988, 263 (27), 13549-13556.
68. Saito, H.; Richardson, C. C., Genetic analysis of gene 1.2 of bacteriophage T7: isolation of a mutant of Escherichia coli unable to support the growth of T7 gene 1.2 mutants. J Virol 1981, 37 (1), 343-351.
69. Airen, I. O. Genome-wide Functional Genomic Analysis for Physiological Investigation and Improvement of Cell-Free Protein Synthesis. Standford University, 2011.
70. Kim, J.; Copeland, C. E.; Seki, K.; Vogeli, B.; Kwon, Y. C., Tuning the Cell-Free Protein Synthesis System for Biomanufacturing of Monomeric Human Filaggrin. Front Bioeng Biotechnol 2020, 8, 590341.
71. Noireaux, J. S. V., Efficient cell-free expression with the endogenous E. Coli RNA polymerase and sigma factor 70. Journal of Biological Engineering 2010, 4 (8).
72. Yim, S. S.; Johns, N. I.; Park, J.; Gomes, A. L.; McBee, R. M.; Richardson, M.; Ronda, C.; Chen, S. P.; Garenne, D.; Noireaux, V.; Wang, H. H., Multiplex transcriptional characterizations across diverse bacterial species using cell-free systems. Mol Syst Biol 2019, 15 (8), e8875.
73. Li, J.; Wang, H.; Kwon, Y. C.; Jewett, M. C., Establishing a high yielding streptomyces-based cell-free protein synthesis system. Biotechnol Bioeng 2017, 114 (6), 1343-1353.
74. Jakociune, D.; Moodley, A., A Rapid Bacteriophage DNA Extraction Method. Methods Protoc 2018, 1 (3).
75. Chen, P.; Zhou, J.; Wan, Y.; Liu, H.; Li, Y.; Liu, Z.; Wang, H.; Lei, J.; Zhao, K.; Zhang, Y.; Wang, Y.; Zhang, X.; Yin, L., A Cas12a ortholog with stringent PAM recognition followed by low off-target editing rates for genome editing. Genome Biol 2020, 21 (1), 78.
76. Liu, Y.; Huang, H.; Wang, H.; Zhang, Y., A novel approach for T7 bacteriophage genome integration of exogenous DNA. J Biol Eng 2020, 14, 2.
77. Hay, M.; Li, Y. M.; Ma, Y., RNA extraction of Escherichia coli grown in Lysogeny Broth for use in RT-qPCR. JEMI Methods 2017, 1, 1-6.
78. Livak, K. J.; Schmittgen, T. D., Analysis of relative gene expression data using real-time quantitative PCR and the 2(−Delta Delta C(T)) Method. Methods 2001, 25 (4), 402-8.
79. Zhou, K.; Zhou, L.; Lim, Q.; Zou, R.; Stephanopoulos, G.; Too, H. P., Novel reference genes for quantifying transcriptional responses of Escherichia coli to protein overexpression by quantitative PCR. BMC Mol Biol 2011, 12, 18.
80. Kim, J.; Copeland, C. E.; Padumane, S. R.; Kwon, Y. C., A Crude Extract Preparation and Optimization from a Genomically Engineered Escherichia coli for the Cell-Free Protein Synthesis System: Practical Laboratory Guideline. Method Protocol 2019, 2 (3).

1. A genetically modified bacterial host cell comprising
a non-endogenous expression vector that comprises a translation initiation factor IF-3 (infC) nucleic acid sequence of SEQ ID NO: 39, an OxyS nucleic acid sequence of SEQ ID NO: 41, and/or a CyaR nucleic acid sequence of SEQ ID NO: 42; and/or
a vector that includes a nucleic acid sequence encoding a CRISPR enzyme and a vector that includes a nucleic acid sequence encoding a crRNA that specifically targets a promoter region located upstream of a transcription start site (TSS) of recC or a recC coding region located downstream of a transcription start site (TSS) of recC, optionally wherein the vector including the nucleic acid sequence encoding the CRISPR enzyme and the vector including the nucleic acid sequence encoding the cRNA are the same; and/or
a deletion or mutation in (a) a promoter region located upstream of a transcription start site (TSS) of recC, or (b) a recC coding region located downstream of a transcription start site (TSS) of recC, wherein the mutation or deletion represses transcription of recC, optionally wherein the mutation is a nonsense mutation, a frameshift mutation, or a missense mutation or the deletion is a 10-100 base pair deletion located upstream or downstream of the transcription start site (TSS) of recC.

2. (canceled)

3. (canceled)

4. (canceled)

5. (canceled)

6. (canceled)

7. (canceled)

8. The genetically modified bacterial host cell of claim 1, wherein the non-endogenous expression vectors comprising the infC nucleic acid sequence, the OxyS nucleic acid sequence and/or the CyaR nucleic acid sequence are the same or distinct.

9. (canceled)

10. The genetically modified bacterial host cell of claim 1, wherein the non-endogenous expression vector comprising the infC nucleic acid sequence, and/or the non-endogenous expression vector comprising the OxyS nucleic acid sequence, and/or the non-endogenous expression vector comprising the CyaR nucleic acid sequence is a plasmid, a cosmid, a bacmid, a bacterial artificial chromosome (BAC), or a viral vector.

11. The genetically modified bacterial host cell of claim 1, wherein the non-endogenous expression vector comprising the infC nucleic acid sequence, and/or the non-endogenous expression vector comprising the OxyS nucleic acid sequence, and/or the non-endogenous expression vector comprising the CyaR nucleic acid sequence is operably linked to an expression control sequence, optionally wherein the expression control sequence is an inducible promoter, a constitutive promoter, an endogenous promoter, or a heterologous promoter.

12. (canceled)

13. (canceled)

14. (canceled)

15. The genetically modified bacterial host cell of claim 1, wherein the nucleic acid sequence encoding the CRISPR enzyme is operably linked to an inducible promoter and/or the nucleic acid sequence encoding the crRNA is operably linked to a constitutive promoter; or wherein the CRISPR enzyme is a nuclease-deficient Francisella novicida Cas12a (dFnCas12a) and/or wherein the nucleic acid sequence encoding the crRNA is selected from the group consisting of SEQ ID NO: 86, SEQ ID NO: 87, SEQ ID NO: 88 and SEQ ID NO: 89.

16. (canceled)

17. (canceled)

18. (canceled)

19. (canceled)

20. (canceled)

21. The genetically modified bacterial host cell of claim 1, wherein the genetically modified bacterial host cell is an E. coli cell.

22. A cell lysate obtained from the genetically modified bacterial host cell of claim 1, wherein the cell lysate comprises an effective amount of transcription/translation (TXTL) machinery that is configured to synthesize bacteriophage under cell-free conditions, optionally wherein the cell lysate is prepared using one or more of French-press cell lysis, sonication, runoff reactions, or lysate dialysis.

23. (canceled)

24. An in vitro method for synthesizing bacteriophage virions comprising
contacting a bacteriophage genome with the cell lysate of claim 22, and an energy buffer in vitro to obtain a reaction mixture, and
incubating the reaction mixture under conditions to produce viable phage virions,
wherein the energy buffer comprises canonical amino acids, phosphoenol pyruvate (PEP), nucleoside triphosphates (NTPs), cofactors, and coenzymes.

25. The method of claim 24, wherein the energy buffer further comprises one or more of a polyethylene glycol (PEG) polymer, deoxynucleotide triphosphates (dNTPs), and stabilizers, optionally wherein the PEG polymer comprises PEG-8000 or PEG-6000; or
wherein the energy buffer further comprises one or more of Mg-glutamate, K-glutamate, tRNA, cAMP, folinic acid, spermidine, 3-PGA, HEPES, Nicotinamide adenine dinucleotide (NAD), coenzyme A (CoA), DTT, and maltodextrin.

26. (canceled)

27. (canceled)

28. The method of claim 24, wherein the reaction mixture further comprises a vector including a nucleic acid sequence encoding a reporter gene, wherein the nucleic acid sequence encoding the reporter gene is operably linked to a promoter that is responsive to a RNA polymerase (RNAP) encoded by the bacteriophage genome, wherein the RNAP is selected from among SP6 RNA Polymerase, T3 RNA Polymerase and T7 RNA polymerase; or wherein the reporter gene encodes a bioluminescent protein, a fluorescent protein, or a chemiluminescent protein.

29. (canceled)

30. (canceled)

31. The method of claim 28, wherein the bioluminescent protein is Aequorin, firefly luciferase, Renilla luciferase, red luciferase, luxAB, or nanoluciferase; or
wherein the chemiluminescent protein is β-galactosidase, horseradish peroxidase (HRP), or alkaline phosphatase; or
wherein the fluorescent protein is sfGFP, TagBFP, Azurite, EBFP2, mKalamal, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOx, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherryl, PATagRFP, Kaede (green), Kaede (red), KikGR1 (green), KikGR1 (red), PS-CFP2, PS-CFP2, mEos2 (green), mEos2 (red), PSmOrange, or Dronpa.

32. (canceled)

33. (canceled)

34. The method of claim 24, wherein the bacteriophage genome is derived from a naturally occurring bacteriophage, or a genetically engineered bacteriophage and/or wherein the bacteriophage genome is derived from a bacteriophage selected from among T7 phage, T7-like phage, Lambda, K1E, T3, T5, T4, or PhiX174.

35. The method of claim 24, wherein the phage virions show increased efficiency of plating (EOP) relative to phage virions obtained with cell-free bacteriophage synthesis (CFBS) in a control lysate obtained from a wild-type bacterial cell, wherein the wild-type bacterial cell and the genetically modified bacterial host cell are the same species.

36. A kit comprising one or more expression vectors comprising at least one nucleic acid sequence selected from the group consisting of SEQ ID NO: 39, SEQ ID NO: 41 and SEQ ID NO: 42, and instructions for using the same to prepare donor bacterial host cell lysates for cell-free bacteriophage synthesis (CFBS).

37. The kit of claim 36, wherein the at least one nucleic acid sequence is operably linked to an inducible promoter, a constitutive promoter, an endogenous promoter, or a heterologous promoter.

38. A kit comprising one or more expression vectors comprising at least one nucleic acid sequence encoding a crRNA that specifically targets a promoter region located upstream of a transcription start site (TSS) of recC or a recC coding region located downstream of a transcription start site (TSS) of recC, and instructions for using the same to prepare donor bacterial host cell lysates for cell-free bacteriophage synthesis (CFBS), optionally wherein the one or more expression vectors further comprise a nucleic acid sequence encoding a CRISPR enzyme.

39. The kit of claim 38, wherein the nucleic acid sequence encoding the CRISPR enzyme is operably linked to an inducible promoter and/or the at least one nucleic acid sequence encoding the crRNA is operably linked to a constitutive promoter.

40. A kit comprising the genetically modified bacterial host cell of claim 1, and instructions for using the same to prepare donor bacterial host cell lysates for cell-free bacteriophage synthesis (CFBS).

41. The kit of claim 36 further comprising one or more of polyethylene glycol (PEG) polymers, deoxynucleotide triphosphates (dNTPs), stabilizers, and an energy buffer comprising canonical amino acids, phosphoenol pyruvate (PEP), nucleoside triphosphates (NTPs), cofactors, and coenzymes.

42. The kit of claim 41, further comprising a vector including a nucleic acid sequence encoding a reporter gene, wherein the nucleic acid sequence encoding the reporter gene is operably linked to a promoter that is responsive to a phage RNA polymerase (RNAP).
